SEQUENCE LISTING (21 sequences)

SEQ ID NO: 1 - protein, 23 aa, Artificial
  Synthetic fusogenic peptide.
GLFHAIAHFIHGGWHGLIHGWYG

SEQ ID NO: 2 - protein, 5 aa, Artificial
  Synthetic peptide targeting ligand
RGDFK

SEQ ID NO: 3 - protein, 5 aa, Artificial
  Synthetic peptide targeting ligand
RGDFC

SEQ ID NO: 4 - protein, 5 aa, Artificial
  Synthetic peptide targeting ligand
RGDYC

SEQ ID NO: 5 - protein, 12 aa, Artificial
  Synthetic peptide targeting ligand
YHWYGYTPQNVI

SEQ ID NO: 6 - protein, 9 aa, Artificial
  Synthetic peptide targeting ligand
GSGKCCYSL

SEQ ID NO: 7 - protein, 8 aa, Artificial
  Synthetic peptide targeting ligand
QWAVGHML

SEQ ID NO: 8 - protein, 8 aa, Artificial [sequence omitted]

SEQ ID NO: 9 - protein, 6 aa, Artificial
  Synthetic peptide targeting ligand
RRPYIL

SEQ ID NO: 10 - protein, 19 aa, Artificial
  Synthetic peptide targeting ligand
RRPYILQLYENKPRRPYIL

SEQ ID NO: 11 - protein, 5 aa, Artificial
  Synthetic peptide targeting ligand
RDGRC

SEQ ID NO: 12 - protein, 5 aa, Artificial
  Synthetic peptide targeting ligand
RGDFK

SEQ ID NO: 13 - protein, 14 aa, Artificial
  Synthetic peptide targeting ligand
KKKKKKKKRGDRGD

SEQ ID NO: 14 - protein, 13 aa, Artificial
  Synthetic peptide targeting ligand
NNNGPLGRGRGDK

SEQ ID NO: 15 - protein, 11 aa, Artificial
  Synthetic peptide targeting ligand
NNNRGDFFFFC

SEQ ID NO: 16 - DNA, 20 bp, Artificial
  PCR primer
gatcgtcctgcagtctctcc

SEQ ID NO: 17 - DNA, 20 bp, Artificial
  PCR primer
aactgtgttcccgaggtgtc

SEQ ID NO: 18 - DNA, 21 bp, Artificial
  PCR primer
ccgccttctgtaatggtttga

SEQ ID NO: 19 - DNA, 21 bp, Artificial
  PCR primer
gggcagctgtatgatctggaa

SEQ ID NO: 20 - DNA, 21 bp, Artificial
  PCR primer
caacagcaactcccactcttc

SEQ ID NO: 21 - DNA, 22 bp, Artificial
  PCR primer
ggtccagggtttcttactcctt

(19) United States
(12) Patent Application Publication
(10) Pub. No.: US 2023/0241000 A1
(43) Pub. Date: Aug. 3, 2023

(54) GSK3 INHIBITOR-LOADED NANO FORMULATIONS AS A CANCER IMMUNOTHERAPEUTIC

(71) Applicant: The Regents of the University of California, Oakland, CA (US)

(72) Inventors: Andre E. Nel, Sherman Oaks, CA (US); Huan Meng, Los Angeles, CA (US); Sean Allen, Culver City, CA (US)

(73) Assignee: The Regents of the University of California, Oakland, CA (US)

(21) Appl. No.: 18/013,508

(22) PCT Filed: Jun. 29, 2021
(86) PCT No.: PCT/US2021/039583

§ 371 Date: Dec. 28, 2022

Related U.S. Application Data
(60) Provisional application No. 62/705,509, filed on Jun. 30, 2020.

(51) Int. Cl. A61K 9/51; A61K 9/127; A61K 31/497; A61K 31/5377; A61K 31/5517; A61K 31/55; A61K 47/24; A61K 31/4745; A61K 47/68; A61K 31/136; A61K 31/7068; A61K 31/704; A61K 47/64; A61P 1/18; A61P 1/00; A61P 11/00; A61P 35/00

(52) U.S. Cl. CPC A61K 9/5115; A61K 9/1271; A61K 31/497; A61K 31/5377; A61K 31/5517; A61K 31/55; A61K 47/24; A61K 31/4745; A61K 47/6851; A61K 31/136; A61K 31/7068; A61K 31/704; A61K 47/64; A61P 1/18; A61P 1/00; A61P 11/00; A61P 35/00

(57) ABSTRACT

In various embodiments nanoparticle drug delivery vehicles are provided herein for the effective delivery of a GSK3 inhibitor. In certain embodiments the drug delivery vehicle comprises a nanoparticle comprising one or more cavities disposed within the nanoparticle and an outside surface where the one or more cavities are in fluid communication the outside surface of the nanoparticle; a GSK3 inhibitor disposed within said one or more cavities; and a lipid bilayer disposed on the surface of said nanoparticle where said lipid bilayer fully encapsulates said nanoparticle.

### CROSS-REFERENCE TO RELATED APPLICATIONS

This application claims benefit of and priority to U.S. Ser. No. 62/705,509, filed on Jun. 30, 2020, which is incorporated herein by reference in its entirety for all purposes.

### STATEMENT OF GOVERNMENTAL SUPPORT

This invention was made with government support under Grant Number CA198846, awarded by the National Institutes of Health. The government has certain rights in the invention.

### INCORPORATION BY REFERENCE OF SEQUENCE LISTING PROVIDED AS A TEXT FILE

This application contains references to nucleic acid sequences that have been submitted concurrently herewith as the sequence listing text file “UCLA-P217PUS_ST25.txt”, file size 4.29 kb, created on Jun. 29, 2020, which is incorporated by reference in its entirety pursuant to 37 C.F.R. 1.52(e)(5).

### BACKGROUND

Despite considerable advances in the past decade, cancer remains a significant global health burden and a major cause of mortality. While there has been a major paradigm shift in cancer treatment through the introduction of immune checkpoint blocking antibodies (e.g., monoclonal antibodies against CTLA4 and PD1, proteins responsible for immune checkpoint signaling that reduces the immune response in the tumor microenvironment), only some cancers respond and even among those only ˜20% of patients are responsive to antibody therapy (1). Differences in the response rate to cancer immunotherapy reflect variances in the pre-existing immune inflammatory status (e.g., presence of cytotoxic T-lymphocytes) of the cancers as well as the heterogeneous immune landscape of the tumor microenvironment (TME). This includes the presence of any number of immune escape mechanisms that may subdue the burgeoning immune response, including immune checkpoint pathways (e.g., CTLA-4 or the PD-1/PD-L1 axis), marginalization and exclusion of T-cells from the cancer site, or the recruitment of immunosuppressive cells such as myeloid derived suppressor cells to the tumor stroma (2-4). An additional challenge to the use of antibodies is relatively large molecular weight of immunoglobulins and their participation in Fc-mediated binding interactions that can impact their biodistribution in comparison to small molecules (5). Immune checkpoint inhibitor antibodies can also lead to serious treatment side effects such as the initiation of allergic responses or systemic inflammatory responses, collectively known as immune-related adverse events (6). Moreover, the production of monoclonal antibodies is challenging, prone to batch-to-batch variations, and expensive to produce. To date, the development of ‘generic’ biologics has been hindered by quality control issues and difficulty to demonstrate bio-similarity (7). A key question therefore becomes whether there are alternatives to checkpoint receptor blocking antibodies or alternative ways to interfere in immune checkpoint pathways.

One potential solution to these challenges, would be to develop small molecule inhibitors of the immune checkpoint pathways, e.g., the PD-1/PD-L1 axis. PD-L1 is a transmembrane protein expressed on multiple cells including antigen presenting cells (APC), tumor cells, and stromal cells in the TME (8). Its binding partner, the transmembrane protein PD-1, is predominantly expressed in antigen-specific T-cells and pro-B cells. PD-L1 binding to PD-1 expressed on exhausted CD8⁺ T-cells can interfere in signal transduction by the T-cell antigen receptor (TCR) and hence with tumor cell killing (FIG. 1, panel A) (8). However, with the advent of immune checkpoint blockade through anti-PD-1 or anti-PD-L1 antibodies, it is possible to prevent immune suppression by the PD-1/PD-L1 axis, thereby restoring TCR signal transduction and the ability for activated T cells to commence cytotoxic tumor cell killing (9). While some work has been performed on macrocyclic peptides and other compounds capable of interfering in the binding of PD-1 to PD-L1, a significant recent advance has been the demonstration that inhibition of glycogen synthase kinase 3 (GSK3) can inhibit the expression of PD-1 in T-cells (10). GSK3, a signaling hub protein at the intersection of several important intracellular signaling pathways, including in the post-TCR signaling cascade, is a well-established drug target for which over 20 compounds have been developed to inhibit this serine-threonine kinase (11, 12). More specifically, the use of the compound SB415286 to inhibit GSK3 was found to increase the expression of T-bet, a master transcriptional regulator in T-cells that have the capability of interfering in PD-1 expression (FIG. 1, panel B) (10, 13, 14). Interference in PD-1 expression was shown to reverse the inhibition of cytotoxic T-cell responses in the mouse B16 melanoma cancer model, with an equal effectiveness as anti-PD-1 antibodies (14). Thus, small molecule inhibitors (SMI) of GSK3 offer the potential of being used as a surrogate or to augment the effect of immune checkpoint blocking antibodies, in addition to emerging use applications for Alzheimer's disease, diabetes and certain cancers.

While presenting theoretical advantages over the use of antibodies, GSK3 small molecule inhibitors face challenges from a drug development perspective (12, 15). This includes possible issues of systemic or off-target toxicity as a result of the α- or β-isoforms of this kinase being involved in multiple signaling pathways, with the possibility that GSK3 inhibition could exert pleiotropic or potential life-threatening side effects. Indeed, toxicity concerns in preclinical and phase I trials resulted in the abandonment of a GSK3 inhibitor to treat Alzheimer's disease (12, 15). Moreover, from the perspective of cancer treatment, GSK3 inhibitors would frequently need to be combined with other drugs (16-18), which requires consideration of their pharmacokinetics and the possibility of drug/drug interactions.

### SUMMARY

In various embodiments nanoparticle drug delivery vehicles are provided herein for the effective delivery of a GSK3 inhibitor. In certain embodiments the drug delivery vehicle comprises a nanoparticle containing one or more cavities within which one or more GSK3 inhibitors are disposed. The nanoparticle is covered (coated) with a lipid bilayer that encapsulates and effectively seals the drug delivery vehicle. In certain embodiments the nanoparticle comprises a solid nanoparticle disposed within and fully encapsulated by a lipid bilayer where the cargo (e.g., GSK3 inhibitor) is adsorbed or covalently- or ionically bound to the surface of the nanoparticle. In certain embodiments the drug delivery vehicle comprises a liposome without a nanoparticle core.

Various embodiments contemplated herein may include, but need not be limited to, one or more of the following:

Embodiment 1: A drug delivery vehicle for the delivery of a GSK3 inhibitor, wherein:
said drug delivery vehicle comprises:
   a nanoparticle comprising one or more cavities disposed within said nanoparticle and an outside surface where said one or more cavities are in fluid communication the outside surface of said nanoparticle;
   a GSK3 inhibitor disposed within said one or more cavities; and
   a lipid bilayer disposed on the surface of said nanoparticle where said lipid bilayer fully encapsulates said nanoparticle; or
said drug delivery vehicle comprises:
   a solid nanoparticle;
   a GSK3 inhibitor disposed on the surface of said nanoparticle and/or intermixed within said nanoparticle; and
   a lipid bilayer disposed on the surface of said nanoparticle where said lipid bilayer fully encapsulates said nanoparticle; or
said drug delivery vehicle comprises a liposome comprising a lipid bilayer.

Embodiment 2: The drug delivery vehicle of embodiment 1, wherein said GSK3 inhibitor comprises a weak basic GS3K inhibitor.

Embodiment 3: The drug delivery vehicle of embodiment 2, wherein said GS3K inhibitor is selected from the group consisting of AZD2858, AZD1080, LY2090314, and 1-Azakenpaullone.

Embodiment 4: The drug delivery vehicle of embodiment 3, wherein said GS3K inhibitor comprises AZD1080.

Embodiment 5: The drug delivery vehicle according to any one of embodiments 1-4, wherein said drug delivery vehicle comprises: a nanoparticle comprising one or more cavities disposed within said nanoparticle and an outside surface where said one or more cavities are in fluid communication the outside surface of said nanoparticle; a GSK3 inhibitor disposed within said one or more cavities; and a lipid bilayer disposed on the surface of said nanoparticle where said lipid bilayer fully encapsulates said nanoparticle.

Embodiment 6: The drug delivery vehicle of embodiment 5, said nanoparticle comprise a single cavity.

Embodiment 7: The drug delivery vehicle of embodiment 6, wherein said nanoparticle comprises a nanobowl.

Embodiment 8: The drug delivery vehicle of embodiment 6, wherein said nanoparticle comprises a hollow nanosphere.

Embodiment 9: The drug delivery vehicle of embodiment 5, wherein said nanoparticle comprises a plurality of cavities.

Embodiment 10: The drug delivery vehicle of embodiment 9, wherein said nanoparticle comprises a porous inorganic nanoparticle, a metal-organic framework nanoparticle, or a porous organic nanoparticle.

Embodiment 11: The drug delivery vehicle of embodiment 10, wherein said nanoparticle comprise a porous inorganic nanoparticle.

Embodiment 12: The drug delivery vehicle of embodiment 11, wherein said nanoparticle comprise a porous silica nanoparticle, a porous calcium carbonate nanoparticle, a porous carbon nanoparticle, a hollow core Fe₃O₄ nanoparticle, or a porous calcium phosphate nanoparticle.

Embodiment 13: The drug delivery vehicle of embodiment 12, wherein said nanoparticle comprises a porous silica nanoparticle.

Embodiment 14: The drug delivery vehicle of embodiment 13, wherein said nanoparticle comprises a mesoporous silica nanoparticle (MSN), a mesoporous organosilica nanoparticle (MONs), or a periodic mesoporous organosilica (PMO) nanoparticle.

Embodiment 15: The drug delivery vehicle of embodiment 14, wherein said nanoparticle comprises a mesoporous silica nanoparticle (MSN).

Embodiment 16: The drug delivery vehicle of embodiment 15, wherein said nanoparticle comprises undoped and unfunctionalized silica.

Embodiment 17: The drug delivery vehicle according to any one of embodiments 14-15, wherein said nanoparticle comprises a mesoporous silica/hydroxyapatite (MSNs/HAP) hybrid nanoparticle.

Embodiment 18: The drug delivery vehicle according to any one of embodiments 14-15, wherein said nanoparticle comprises a cleavable silsesquioxane, or a bridged silsesquioxane (BS).

Embodiment 19: The drug delivery vehicle according to any one of embodiments 14-15, wherein said nanoparticle comprises an inorganically doped silica.

Embodiment 20: The drug delivery vehicle of embodiment 19, wherein said nanoparticle comprises a calcium-, iron-, manganese-, or zirconium-doped silica.

Embodiment 21: The drug delivery vehicle according to any one of embodiments 14-15, wherein said nanoparticle comprises an imine-doped silica.

Embodiment 22: The drug delivery vehicle of embodiment 12, wherein said nanoparticle comprises a mesoporous calcium carbonate nanoparticle.

Embodiment 23: The drug delivery vehicle of embodiment 12, wherein said nanoparticle comprises a mesoporous calcium phosphate nanoparticle.

Embodiment 24: The drug delivery vehicle of embodiment 10, wherein said nanoparticle comprises a porous biocompatible polymer.

Embodiment 25: The drug delivery vehicle of embodiment 24, wherein said nanoparticle comprise a porous biocompatible polymer selected from the group consisting of polymers of the polyaryletherketone (PAEK) family (e.g., polyether ether ketone (PEEK), carbon reinforced PEEK, polyether ketone ketone (PEKK), PEKEKK (polyetherketoneetherketoneketone), polyaryletherketone (PAEK), polyetherketone (PEK), Polyetherketone Etherketone Ketone (PEKEKK), and the like), polycaprolactone (PCL), polylactic acid (PLA), polyglycolic acid (PGA), polyphenylene, self-reinforced polyphenylene, polyphenylsulphone, polysulphone, polyethylene terephthalate (PET), polyethylene, polyurethane, oligocarbonatedimethacrylate (OCM-2) porous polymer, carbonate- and phthalate-containing dimethacrylates, and the like.

Embodiment 26: The drug delivery vehicle of embodiment 24, wherein said nanoparticle comprises a hydrogel.

Embodiment 27: The drug delivery vehicle of embodiment 26, wherein said hydrogel comprises a hydrogel formed from one or more materials selected from the group consisting of poly(N-isopropylacrylamide) (PNIPA), poly(N-isopropylacrylamide-co-1-vinylimidazole) (PNIPA-VI), poly(acrylamide) (PAAm), poly(acrylamide), poly(N,N-dimethylacrylamide), poly(N,N-diethylacrylamide), poly(1-vinylimidazole), poly(sodium acrylate), poly(sodium methacrylate), poly(2-hydroxyethylmethacrylate) (HEMA), poly(N,N-dimethylaminoethyl methacrylate) (DMAEMA), poly(N-[tris(hydroxymethyl)methyl]acrylamide), poly(1-(3-methacryloxy)propylsulfonic acid) (sodium salt), poly(allylamine), poly(N-acryloxysuccinimide), poly(N-vinylcaprolactam), poly(1-vinyl-2-pyrrolidone), poly(2-acrylamido-2-methyl-1-propanesulfonic acid) (sodium salt), poly((3-acrylamidopropyl) trimethylammonium chloride), and poly(diallyldimethylammonium chloride).

Embodiment 28: The drug delivery vehicle of embodiment 10, wherein said nanoparticle comprises a metal organic framework (MOF).

Embodiment 29: The drug delivery vehicle of embodiment 28, wherein said nanoparticle comprises a metal organic framework selected from the group consisting of zeolitic imidazolate frameworks (ZIFs), Universitetet i Oslo (University of Oslo) frameworks (UiOs), and (Materials of Institut Lavoisier frameworks (MILs).

Embodiment 30: The drug delivery vehicle of embodiment 29, wherein said nanoparticle comprises a metal organic framework selected from the group consisting of ZIF-8, ZIF-67, ZIF-90, Fe-BTC, HKUST-1, and MIL-53, MIL-89, MIL-88A, MIL-100, UiO-66, UiO-66-NH₂, MOF-801, MOF-804, Fe-NDC-M, MOF-1201, MOF-1203, and Fe-NDC-O MOFs.

Embodiment 31: The drug delivery vehicle of embodiment 30, wherein said nanoparticle comprises a MIL-88A MOF.

Embodiment 32: The drug delivery vehicle of embodiment 30, wherein said nanoparticle comprises a ZIF-8 MOF.

Embodiment 33: The drug delivery vehicle of embodiment 30, wherein said nanoparticle comprises a UiO-66 MOF, or a UiO-66-NH₂ MOF.

Embodiment 34: The drug delivery vehicle according to any one of embodiments 1-33, wherein said nanoparticle has an average pore size that ranges from about 1 to about 20 nm, or from about 1 to about 10 nm, or from about 1 to about 5 nm, or from about 1 to about 4 nm, or from about 1 to about 3 nm, or from about 2 to about 3 nm.

Embodiment 35: The drug delivery vehicle according to any one of embodiments 1-4, wherein said drug delivery vehicle comprises:
a solid nanoparticle;
a GSK3 inhibitor disposed on the surface of said nanoparticle and/or intermixed within said nanoparticle; and
a lipid bilayer disposed on the surface of said nanoparticle where said lipid bilayer fully encapsulates said nanoparticle.

Embodiment 36: The drug delivery vehicle of embodiment 35, wherein said nanoparticle is selected from the group consisting of a metal nanoparticle, or a biocompatible polymer nanoparticle.

Embodiment 37: The drug delivery vehicle of embodiment 36, wherein said nanoparticle comprises a zinc oxide nanoparticle, a gold nanoparticle, a silver nanoparticle, an aluminum hydroxide nanoparticle, a silica nanoparticle, a core-shell Fe₃O—SiO₂ nanoparticle, a core-shell NaYbF4:Tm-NaYF₄ upconverting nanoparticle, or a LiYF₄:Tm/Yb nanocrystal.

Embodiment 38: The drug delivery vehicle of embodiment 36, wherein said nanoparticle comprises a biocompatible polymer.

Embodiment 39: The drug delivery vehicle of embodiment 38, wherein said nanoparticle comprises a biocompatible polymer selected from the group consisting of poly(lactic-co-glycolic acid) (PLGA), Poly(glycolic acid) (PGA), Poly(lactic acid) (PLA), Poly(caprolactone) (PCL), Poly(butylene succinate), Poly(trimethylene carbonate), Poly(p-dioxanone), Poly(butylene terephthalate), Poly(ester aminde) (HYBRANE®), polyurethane, Poly[(carboxyphenoxy) propane-sebacic acid], Poly[bis(hydroxyethyl) terephthalate-ethyl orthophosphorylate/terephthaloyl chloride], Poly(β-hydroxyalkanoate), Poly(hydroxybutyrate), Poly(hydroxybutyrate-co-hydroxyvalerate), and chitosan.

Embodiment 40: The drug delivery vehicle according to any one of embodiments 1-4, wherein said drug delivery vehicle comprises a liposome that comprises a lipid bilayer.

Embodiment 41: The drug delivery vehicle according to any one of embodiments 1-40, wherein said drug delivery vehicles have an average hydrodynamic diameter ranging from about 30 nm up to about 300 nm, or from about 30 up to about 200 nm, or from about 30 up to about 170 nm, or from about 30 nm up to about 150 nm, or from about 30 up to about 100 nm, or from about 30 up to about 80 nm, or from about 30 up to about 70 nm, or from about 40 up to about 70 nm by DLS.

Embodiment 42: The drug delivery vehicle of embodiment 41, wherein said drug delivery vehicles have an average hydrodynamic diameter ranging from about 70 nm up to about 165 nm by DLS by DLS.

Embodiment 43: The nanoparticle drug carrier according to any one of embodiments 1-42, wherein said lipid bilayer comprises a phospholipid, and cholesterol (CHOL) and/or a cholesterol derivative.

Embodiment 44: The nanoparticle drug carrier of embodiment 43, wherein said lipid bilayer comprises a phospholipid and cholesterol (CHOL).

Embodiment 45: The nanoparticle drug carrier according to any one of embodiments 43-44, wherein said phospholipid comprises a saturated fatty acid with a C14-C20 carbon chain, and/or an unsaturated fatty acid with a C14-C20 carbon chain, and/or a natural lipid comprising a mixture of fatty acids with C12-C20 carbon chains.

Embodiment 46: The nanoparticle drug carrier of embodiment 45, wherein said phospholipid comprises one or more phospholipids selected from the group consisting of distearoylphosphatidylcholine (DSPC), phosphatidylcholine (DPPC), 1,2-dimyristoleoyl-sn-glycero-3-phosphocholine (DMPC), 1,2-dioleoyl-sn-glycero-3-phosphocholine (DOPC), 1,2-distearoyl-sn-glycero-3-phospho-rac-glycerol (DSPG), 1,2-dipalmitoyl-sn-glycero phosphoglycerol (DPPG), 1,2-dieicosenoyl-sn-glycero-3-phosphocholine, and diactylphosphatidylcholine (DAPC), and dipalmitoyl phosphatidylethanolamine.

Embodiment 47: The nanoparticle drug carrier of embodiment 45, wherein said phospholipid comprises a natural lipid selected from the group consisting of egg phosphatidylcholine (egg PC), and soy phosphatidylcholine (soy PC).

Embodiment 48: The nanoparticle drug carrier of embodiment 45, wherein said phospholipid comprises distearoylphosphatidylcholine (DSPC).

Embodiment 49: The nanoparticle drug carrier according to any one of embodiments 43-48, wherein said lipid bilayer comprises an mPEG phospholipid with a phospholipid C14-C18 carbon chain, and a PEG molecular weight ranging from about 350 Da to 5000 Da.

Embodiment 50: The nanoparticle drug carrier of embodiment 49, wherein said lipid bilayer comprises dipalmitoyl phosphatidylethanolamine grafted poly(ethylene glycol) (PE-PEG).

Embodiment 51: The nanoparticle drug carrier of embodiment 50, wherein said PE-PEG comprises PE-PEG_{2K}.

Embodiment 52: The nanoparticle drug carrier of embodiment 50, wherein said PE-PEG comprises PE-PEG_{5K}.

Embodiment 53: The nanoparticle drug carrier according to any one of embodiments 48-52, wherein said lipid bilayer comprises DPSC, cholesterol, and PE-PEG.

Embodiment 54: The nanoparticle drug carrier of embodiment 53, wherein the ratio of DPSC:cholesterol:PE-PEG ranges from 40-90% DSPC:10%-50% Chol:1%-10% PE-PEG (molar ratio).

Embodiment 55: The nanoparticle drug carrier of embodiment 54, wherein the ratio of DSPC:Chol:PE-PEG is about 60:40:3 molar ratio.

Embodiment 56: The nanoparticle drug carrier according to any one of embodiments 43-55, wherein said lipid bilayer comprises a cholesterol derivative selected from the group consisting of cholesterol hemisuccinate (CHEMS), lysine-based cholesterol (CHLYS), and PEGylated cholesterol (Chol-PEG).

Embodiment 57: The nanoparticle drug carrier of embodiment 56, wherein said lipid bilayer comprises CHEMS.

Embodiment 58: The nanoparticle drug carrier of embodiment 57, wherein said bilayer comprises CHEMS ranging from about 5% (mol percent) up to about 30% total lipid.

Embodiment 59: The nanoparticle drug carrier of embodiment 58, wherein said bilayer comprises about 10% or about 20% CHEMS or about 30% CHEMS or about 40% CHEMS.

Embodiment 60: The nanoparticle drug carrier according to any one of embodiments 1-42, wherein said lipid bilayer comprises a formulation shown in Table 3.

Embodiment 61: The nanoparticle drug carrier according to any one of embodiments 1-60, wherein the GSK3 inhibitor is loaded with a cargo trapping agent (e.g., protonating agent).

Embodiment 62: The nanoparticle drug carrier of embodiment 61, wherein said cargo trapping agent before reaction with the GSK3 inhibitor loaded in the nanoparticle, is selected from the group consisting of citric acid, triethylammonium sucrose octasulfate (TEA₈SOS), (NH₄)₂SO₄, an ammonium salt, a trimethylammonium salt, and a triethylammonium salt.

Embodiment 63: The nanovesicle drug carrier of embodiment 62, wherein said cargo-trapping agent before reaction with said drug is ammonium sulfate.

Embodiment 64: The nanoparticle drug carrier according to any one of embodiments 1-63, wherein said drug carrier comprises an additional therapeutic agent disposed inside of the nanoparticle.

Embodiment 65: The nanoparticle drug carrier of embodiment 64, wherein said additional therapeutic agent comprises an inducer of immunogenic cell death (ICD inducer).

Embodiment 66: The nanoparticle drug carrier of embodiment 65, wherein said ICD inducer comprises a chemotherapeutic agent selected from the group consisting of irinotecan, doxorubicin, oxaliplatin, anthracenedione, bleomycin, bortezomib, cisplatin, daunorubicin, docetaxel, epirubicin, idarubicin, mitoxanthrone, paclitaxel, 82016, and cyclophosphamide.

Embodiment 67: The nanoparticle drug carrier of embodiment 66, wherein said ICD inducer comprises irinotecan.

Embodiment 68: The nanoparticle drug carrier of embodiment 64, wherein said additional therapeutic agent comprise an anticancer agent selected from the group consisting of doxorubicin, epirubicin, pirarubicin, daunorubicin, rubidomycin, valrubicin, amrubicin, irinotecan, topotecan, 10-hydroxycamptothecin, belotecan, rubitecan, vinorelbine, LAQ824, vinblastine, vincristine, homoharringtonine, trabectedin, mitoxantrone, cyclophosphamide, mechlorethamine, temozolomide, 5-fluorouracil, 5′-deoxy-5-fluorouridine, gemcitabine, capecitabine, pazopanib, enzastaurin, vandetanib erlotinib, dasatinib, nilotinib, sunitinib, osimertinib, palbociclib, and ribociclib.

Embodiment 69: The nanoparticle drug carrier of embodiment 68, wherein said additional therapeutic agent comprises mitoxantrone.

Embodiment 70: The nanoparticle drug carrier of embodiment 68, wherein said additional therapeutic agent comprises gemcitabine.

Embodiment 71: The nanoparticle drug carrier of embodiment 68, wherein said additional therapeutic agent comprises doxorubicin.

Embodiment 72: The nanoparticle drug carrier according to any one of embodiments 1-71, wherein said drug carrier comprises a hydrophobic therapeutic agent disposed in the lipid bilayer.

Embodiment 73: The nanoparticle drug carrier of embodiment 71, wherein said hydrophobic therapeutic agent comprises paclitaxel.

Embodiment 74: The nanoparticle drug carrier according to any one of embodiments 1-73, wherein said drug carrier is conjugated to a moiety selected from the group consisting of a targeting moiety, a fusogenic peptide, and a transport peptide.

Embodiment 75: The nanoparticle drug carrier of embodiment 74, wherein said drug carrier is conjugated to a peptide that binds a receptor on a cancer cell or tumor blood vessel.

Embodiment 76: The nanoparticle drug carrier of embodiment 75, wherein said drug carrier is conjugated to an iRGD peptide.

Embodiment 77: The nanoparticle drug carrier of embodiment 75, wherein said drug carrier is conjugated to a targeting ligand shown in Table 5.

Embodiment 78: The nanoparticle drug carrier according to any one of embodiments 74-77, wherein said drug carrier is conjugated to transferrin, and/or ApoE, and/or folate.

Embodiment 79: The nanoparticle drug carrier according to any one of embodiments 74-78, wherein said drug carrier is conjugated to a targeting moiety that comprises an antibody that binds to a cancer marker.

Embodiment 80: The nanoparticle drug carrier of embodiment 79, wherein said drug carrier is conjugated to a targeting moiety that comprises an antibody that binds a cancer marker shown in Table 4.

Embodiment 81: The nanoparticle drug carrier according to any one of embodiments 79-80, wherein said antibody is selected from the group consisting of an intact immunoglobulin, an F(ab)′₂, a Fab, a single chain antibody, a diabody, an affibody, a unibody, and a nanobody.

Embodiment 82: The nanoparticle drug carrier according to any one of embodiments 1-81, wherein said drug carriers in suspension are stable for at least 1 month, or at least 2 months, or at least 3 months, or at least 4 months, or at least 5 months, or at least 6 months when stored at 4° C.

Embodiment 83: The nanoparticle drug carrier according to any one of embodiments 1-82, wherein said nanoparticle drug carrier forms a stable suspension on rehydration after lyophilization.

Embodiment 84: The nanoparticle drug carrier according to any one of embodiments 1-83, wherein said nanoparticle drug carriers, show reduced drug toxicity as compared to free GSK3 inhibitor.

Embodiment 85: The nanoparticle drug carrier according to any one of embodiments 1-84, wherein said nanoparticle drug carrier has colloidal stability in physiological fluids with pH 7.4 and remains monodisperse to allow systemic biodistribution and is capable of entering a disease site by vascular leakage (EPR effect) or transcytosis.

Embodiment 86: The nanoparticle drug carrier drug carrier according to any one of embodiments 1-85, wherein said carrier is colloidally stable.

Embodiment 87: A pharmaceutical formulation comprising:
nanoparticle drug carrier according to any one of embodiments 1-86; and
a pharmaceutically acceptable carrier.

Embodiment 88: The pharmaceutical formulation of embodiment 87, wherein said formulation is an emulsion, dispersion, or suspension.

Embodiment 89: The pharmaceutical formulation of embodiment 88, wherein said suspension, emulsion, or dispersion is stable for at least 1 month, or at least 2 months, or at least 3 months, or at least 4 months, or at least 5 months, or at least 6 months when stored at 4° C.

Embodiment 90: The pharmaceutical formulation according to any one of embodiments 87-89, wherein the nanovesicle drug carriers, and/or the a nanoparticle drug carriers, and/or the a nanomaterial carriers in said formulation show a substantially unimodal size distribution; and/or show a PDI less than about 0.2, or less than about 0.1.

Embodiment 91: The pharmaceutical formulation according to any one of embodiments 87-90, wherein said formulation is formulated for administration via a route selected from the group consisting of intravenous administration, intraarterial administration, intracerebral administration, intrathecal administration, oral administration, aerosol administration, administration via inhalation (including intranasal and intratracheal delivery, intracranial administration via a cannula, and subcutaneous or intramuscular depot deposition.

Embodiment 92: The pharmaceutical formulation according to any one of embodiments 87-90, wherein said formulation is a sterile injectable.

Embodiment 93: The pharmaceutical formulation according to any one of embodiments 87-92, wherein said formulation is a unit dosage formulation.

Embodiment 94: A method of treating a cancer, said method comprising:
administering to a subject in need thereof an effective amount of a nanoparticle drug carrier according to any one of clams 1-86; and/or
a pharmaceutical formulation according to any one of embodiments 87-93.

Embodiment 95: The method of embodiment 94, wherein said method comprises a component of a primary therapy in a chemotherapeutic regimen.

Embodiment 96: The method of embodiment 94, wherein said method comprises an adjunct therapy in a treatment regime that additionally comprises chemotherapy using another chemotherapeutic agent, and/or surgical resection of a tumor mass, and/or radiotherapy.

Embodiment 97: The method according to any one of embodiments 94-96, wherein said composition, a nanovesicle drug carrier, a nanoparticle drug carrier according, and/or nanomaterial carrier is a component in a multi-drug chemotherapeutic regimen.

Embodiment 98: The method according to any one of embodiments 94-97, wherein said cancer comprises a solid tumor.

Embodiment 99: The method of embodiment 98, wherein said cancer comprises a cancer selected from the group consisting of gastric cancer, hepatocellular carcinoma, head and neck squamous cell carcinoma, urothelial carcinoma, cervical cancer, non-small cell lung cancer, and broadly for non-respectable solid tumors with high microsatellite instability (MSI-H) or DNA mismatch repair deficiency.

Embodiment 100: The method according to any one of embodiments 94-97, wherein said cancer comprises pancreatic cancer.

Embodiment 101: The method according to any one of embodiments 94-97, wherein said cancer comprises colorectal cancer.

Embodiment 102: The method according to any one of embodiments 94-97, wherein said cancer comprises lung cancer.

Embodiment 103: The method according to any one of embodiments 94-97, wherein said cancer is a cancer selected from the group consisting of breast cancer, lung cancer, melanoma, pancreas cancer, liver cancer, acute lymphoblastic leukemia (ALL), acute myeloid leukemia (AML), adrenocortical carcinoma, AIDS-related cancers (e.g., Kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, brain and spinal cord tumors, brain stem glioma, central nervous system atypical teratoid/rhabdoid tumor, central nervous system embryonal tumors, central nervous system germ cell tumors, craniopharyngioma, ependymoma, burkitt lymphoma, carcinoid tumors (e.g., childhood, gastrointestinal), cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm's tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sézary syndrome), Hodgkin, non-Hodgkin, primary central nervous system (CNS)), macroglobulinemia, Waldenström, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, Myelogenous Leukemia, Chronic (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

Embodiment 104: The method according to any one of embodiments 94-103, wherein said administration is via a route selected from the group consisting of intravenous administration, intraarterial administration, intracerebral administration, intrathecal administration, oral administration, aerosol administration, administration via inhalation (including intranasal and intratracheal delivery, intracranial administration via a cannula, and subcutaneous or intramuscular depot deposition.

Embodiment 105: The method according to any one of embodiments 94-103, wherein said administration comprises systemic administration via injection or cannula.

Embodiment 106: The method according to any one of embodiments 94-103, wherein said administration is administration to an intra-tumoral or peri-tumoral site.

Embodiment 107: The method according to any one of embodiments 94-106, wherein said mammal is a human.

Embodiment 108: The method according to any one of embodiments 94-106, wherein said mammal is a non-human mammal.

### Definitions

The terms “subject,” “individual,” and “patient” may be used interchangeably and refer to humans, as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for a cancer as described herein. Thus, for example, in certain embodiments the subject is a subject with a cancer (e.g., pancreatic ductal adenocarcinoma (PDAC), breast cancer (e.g., drug-resistant breast cancer), colon cancer, brain cancer, and the like). In certain embodiments the methods described herein are prophylactic and the subject is one in whom a cancer is to be inhibited or prevented. In certain embodiments the subject for prophylaxis is one with a family history of cancer and/or a risk factor for a cancer (e.g., a genetic risk factor, an environmental exposure, and the like).

The term “treat” when used with reference to treating, e.g., a pathology or disease refers to the mitigation and/or elimination of one or more symptoms of that pathology or disease, and/or a delay in the progression and/or a reduction in the rate of onset or severity of one or more symptoms of that pathology or disease, and/or the prevention of that pathology or disease. The term “treat” can refer to prophylactic treatment which includes a delay in the onset or the prevention of the onset of a pathology or disease.

The terms “coadministration” or “administration in conjunction with” or “cotreatment” when used in reference to the coadministration of a first compound (or component) (e.g., a GSK3 inhibitor) and a second compound (or component) (e.g., a different cancer therapeutic) indicates that the first compound (or component) and the second compound (or component) are administered so that there is at least some chronological overlap in the biological activity of first compound and the second compound in the organism to which they are administered. Coadministration can include simultaneous administration or sequential administration. In sequential administration there may even be some substantial delay (e.g., minutes or even hours) between administration of the first compound and the second compound as long as their biological activities overlap. In certain embodiments, the coadministration is over a time frame that permits the first compound and second compound to produce an enhanced therapeutic or prophylactic effect on the organism. In certain embodiments the enhanced effect is a synergistic effect.

The terms “nanocarrier” and “nanoparticle drug carrier” are used interchangeably and refer to a nanostructure having a one or a plurality of cavities, e.g., a porous interior. In various embodiments, the cavities contain a cargo that is to be delivered, e.g., to a target cell. In certain embodiments the nanoparticle is a porous silica nanoparticle (e.g., mesoporous silica nanoparticle or “MSNP”). In certain other embodiments, the nanoparticle is a solid nanoparticle and the cargo can be disposed within (e.g., intermixed with) the material forming the nanoparticle or adsorbed to, or covalently or ionically bound to, the nanoparticle surface). In certain embodiments the nanocarrier comprises a lipid bilayer encasing (or surrounding or enveloping) the particle core. In certain embodiments the nanocarrier is a liposome and the cargo can be disposed within the liposome.

As used herein, the term “lipid” refers to conventional lipids, phospholipids, cholesterol, chemically functionalized lipids for attachment of PEG, pharmaceutically active ingredients, ligands, etc.

As used herein, the terms “lipid bilayer” or “LB” refers to any double layer of oriented amphipathic lipid molecules in which the hydrocarbon tails face inward to form a continuous non-polar phase.

As used herein, the term “selective targeting” or “specific binding” refers to use of targeting ligands on the surface of a drug delivery nanocarrier (e.g., a LB-coated nanoparticle or a liposome). In certain embodiments the targeting ligand(s) are on the surface of a lipid bilayer or LB-coated nanoparticle. Typically, the ligands interact specifically/selectively with receptors or other biomolecular components expressed on the target, e.g., a cell surface of interest. The targeting ligands can include such molecules and/or materials as peptides, antibodies, aptamers, targeting peptides, polysaccharides, and the like.

A coated mesoporous silica nanoparticle, having targeting ligands can be referred to as a “targeted nanoparticle or a targeted drug delivery nanocarrier (e.g., LB-coated nanoparticle or a liposome).

The term “about” or “approximately” as used herein refers to being within an acceptable error range for the particular value as determined by one of ordinary skill in the art, which will depend in part on how the value is measured or determined, i.e. the limitations of the measurement system, i.e. the degree of precision required for a particular purpose, such as a pharmaceutical formulation. For example, “about” can mean within 1 or more than 1 standard deviation, per the practice in the art. Alternatively, “about” can mean a range of up to 20%, preferably up to 10%, more preferably up to 5% and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The term “drug” as used herein refers to a chemical entity of varying molecular size, small and large, naturally occurring or synthetic, that exhibits a therapeutic effect in animals and humans. A drug may include, but is not limited to, an organic molecule (e.g., a small organic molecule), a therapeutic protein, peptide, antigen, or other biomolecule, an oligonucleotide, an siRNA, a construct encoding CRISPR cas9 components and, optionally one or more guide RNAs, and the like.

A “pharmaceutically acceptable carrier” as used herein is defined as any of the standard pharmaceutically acceptable carriers. The pharmaceutical compositions of the subject invention can be formulated according to known methods for preparing pharmaceutically useful compositions. The pharmaceutically acceptable carrier can include diluents, adjuvants, and vehicles, as well as carriers, and inert, non-toxic solid or liquid fillers, diluents, or encapsulating material that does not react with the active ingredients of the invention. Examples include, but are not limited to: phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W [1995] Easton Pa., Mack Publishing Company, 19th ed.) describes formulations that can be used in connection with the drug delivery nanocarrier(s) (e.g., liposomes or nanoparticles encapsulated with a lipid bilayer) described herein.

As used herein, an “antibody” refers to a protein consisting of one or more polypeptides substantially encoded by immunoglobulin genes or fragments of immunoglobulin genes or derived therefrom that is capable of binding (e.g., specifically binding) to a target (e.g., to a target polypeptide). The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains, respectively.

Antibodies exist as intact immunoglobulins or as a number of well characterized fragments produced by digestion with various peptidases. Thus, for example, pepsin digests an antibody below the disulfide linkages in the hinge region to produce F(ab)′₂ a dimer of Fab which itself is a light chain joined to V_{H}-C_{H}1 by a disulfide bond. The F(ab)′₂ may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Certain preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked V_{H}-V_{L} heterodimer which may be expressed from a nucleic acid including V_{H}- and V_{L}-encoding sequences either joined directly or joined by a peptide-encoding linker. Huston, et al. (1988) Proc. Nat. Acad. Sci. USA, 85: 5879-5883. While the V_{H} and V_{L} are connected to each as a single polypeptide chain, the V_{H} and V_{L} domains associate non-covalently. The first functional antibody molecules to be expressed on the surface of filamentous phage were single-chain Fv's (scFv), however, alternative expression strategies have also been successful. For example Fab′ molecules can be displayed on a phage if one of the chains (heavy or light) is fused to g3 capsid protein and the complementary chain exported to the periplasm as a soluble molecule. The two chains can be encoded on the same or on different replicons; the important point is that the two antibody chains in each Fab molecule assemble post-translationally and the dimer is incorporated into the phage particle via linkage of one of the chains to, e.g., g3p (see, e.g., U.S. Pat. No. 5,733,743). The scFv antibodies and a number of other structures converting the naturally aggregated, but chemically separated light and heavy polypeptide chains from an antibody V region into a molecule that folds into a three-dimensional structure substantially similar to the structure of an antigen-binding site are known to those of skill in the art (see e.g., U.S. Pat. Nos. 5,091,513, 5,132,405, and 4,956,778). In certain embodiments antibodies should include all that have been displayed on phage (e.g., scFv, Fv, Fab and disulfide linked Fv (see, e.g, Reiter et al. (1995) Protein Eng. 8: 1323-1331) as well as affibodies, unibodies, and the like.

The term “specifically binds”, as used herein, when referring to a biomolecule (e.g., protein, nucleic acid, antibody, etc.), refers to a binding reaction that is determinative of the presence of a biomolecule in heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

“Two-dimensional materials (2D materials) are materials that do not require a substrate to exist. In other words, they can be isolated as freestanding one atom thick sheets. As a practical matter, this definition can be relaxed to include materials with a thickness of a few atoms (e.g., less than about 10 atoms).

The term “substantially pure isomer” refers to a formulation or composition wherein among various isomers of a compound a single isomer is present at 70%, or greater or at 80% or greater, or at 90% or greater, or at 95% or greater, or at 98% or greater, or at 99% or greater, or said compound or composition comprises only a single isomer of the compound.

The term “immunogenic cell death” or “ICD” refers to a unique form of cell death caused by some cytostatic agents such as anthracyclines (Obeid et al. (2007) Nature Med., 13(1): 54-61), anthracenedione (mitoxantrone, aka MTX), oxaliplatin, irinotecan, and bortezomib, or radiotherapy and/or photodynamic therapy (PDT). Unlike regular apoptosis, which is mostly non-immunogenic or even tolerogenic, immunogenic apoptosis of cancer cells can induce an effective antitumor immune response through activation of dendritic cells (DCs) and consequent activation of specific T cell response (Spisek and Dhodapkar (2007) Cell Cycle, 6(16): 1962-1965). Endoplasmic reticulum (ER) stress, reactive oxygen species (ROS) production and induction of autophagy are key intracellular response pathways that govern ICD (Krysko et al. (2012) Nat. Rev. Canc. 12(12): 860-875). In addition to facilitating tumor cell death that facilitates antigen presentation by dendritic cells, ICD is characterized by secretion or release of damage-associated molecular patterns (DAMPs), which exert additional immune adjuvant effects. Calreticulin (CRT), one of the DAMP molecules, which is normally in the lumen of the ER, is translocated to the surface of dying cell where it functions as an “eat me” signal for phagocytes. Other important surface exposed DAMPs are heat-shock proteins (HSPs), namely HSP70 and HSP90, which under stress conditions are also translocated to the plasma membrane. On the cell surface they have an immunostimulatory effect, based on their interaction with number of antigen-presenting cell (APC) surface receptors like CD91 and CD40 and also facilitate cross-presentation of antigens derived from tumor cells on MHC class I molecule, which then triggers CD8+ T cell activation and expansion. Other important DAMPs, characteristic for ICD are secreted, high-mobility group box 1 (HMGB1) protein and ATP (see, e.g., Apetoh et al. (2007) Nature Med. 13(9): 1050-1059; Ghiringhelli et al. (2009) Nature Med. 15(10): 1170-1178). HMGB1 is considered to be a late apoptotic marker and its release to the extracellular space appears to be required for the optimal release and presentation of tumor antigens to dendritic cells. It binds to several pattern recognition receptors (PRRs) such as Toll-like receptor (TLR) 2 and 4, which are expressed on APCs. The most recently found DAMP released during immunogenic cell death is ATP, which functions as a “find-me” signal for monocytes when secreted and induces their attraction to the site of apoptosis (see, e.g., Garg et al. (2012) EMBO J. 31(5): 1062-1079). ATP binds to purinergic receptors on APCs. An inducer of immunogenic cell death is referred to as an ICD inducer.

### BRIEF DESCRIPTION OF THE DRAWINGS

FIG. 1, panels A-B illustrate interference in PD-1 expression by small molecule GSK3 inhibitors. Panel A: Illustration of the PD-1/PD-L1 signaling axis, which suppresses cytotoxic T-cell tumor cell killing by inhibiting signal transduction by the T-cell antigen receptor (TCR) complex. PD-1 is expressed on “exhausted” T-cells, leading to the recruitment of the SHP2 phosphatase, which interferes in recruitment of signaling components to tyrosine residues in the TCR signal. This prevents the release of cytolytic granules. Constitutionally active GSK3 is responsible for preventing the transcriptional activation of the T-bet promoter (Tbx21). Panel B: Introduction of a GSK3 inhibitor (e.g., by a nanocarrier) allows restoration of T-bet expression, leading to transcriptional interference of the PD-1 promoter (Pdcd1) complex. The disappearance of PD-1 from the cell surface restores TCR signal transduction, allowing tumor cell killing by cytotoxic T-cells. In this sense, the transcriptional suppression of PD-1 (through pharmacological inhibition) exerts the same effect as blocking of the interaction of PD-1 with its ligand by antibodies.

FIG. 2, panels A-B, illustrates remote loading considerations based on the chemical properties of GSK3 inhibitors. Panel A: Illustration of remote loading by the lipid bilayer coated mesoporous silica nanoparticle (a.k.a. silicasome) carrier, requiring the generation of a proton gradient for drug import across the lipid bilayer. Panel B: Scheme demonstrating the chemical criteria to assess the remote loading capacity of commercially available GSK3 inhibitors (FIG. 10). These criteria include compound, molecular weight (MVV), partition coefficient (cLogP), isoelectric point (pI) and solubility coefficient (LogS) at pH 7.4 as illustrated in this Figure.

FIG. 3, panels A-C, shows characterization of GSK3 inhibitor silicasome carriers. Panel A: Representative images to explain large batch synthesis of bare MSNPs. A 20 L sol-gel synthesis of ˜70 nm bare MSNP was prepared as previously described, yielding a 120 g batch of high-quality particles (22). Panel B: Illustration of the subsequent synthesis procedure to obtain lipid-coated MSNPs (silicasomes) for soaking in the trapping agent, ammonium sulfate. Panel C: CryoTEM micrograph of silicasomes after loading of GSK3 inhibitor, AZD1080. Scale bar=100 nm. A schematic illustration of the AZD1080 laden silicasome appears on the right hand side.

FIG. 4, panels A-C, illustrates an in vitro assay to show GSK3 inhibition of Pdcd1 expression. Panel A: Experimental procedure used for assessing anti-CD3 induced Pdcd1 expression and inhibition by GSK3 inhibitors. The expression of (panel B) Pdcd1 and (panel C) Tbx21 was determined by qRT-PCR and reported as log₂-fold change. The negative control was comprised of unstimulated cells, while cells treated with immobilized anti-CD3 antibody only (“aCD3 Only”) represents the positive control. Significance was determined using 2-way ANOVA, *p<0.05 and **p<0.01. Error bars=SD, n=4.

FIG. 5, panels A-D, shows a comparison of the effects of free and encapsulated GSK3 inhibitors and anti-PD1 on MC38 tumor growth and the effect on cytotoxic T-cell responses. The animal treatment is discussed in the method section. Panel A: Tumor volume growth curves starting from the time of inoculation up to sacrifice. Green arrows represent days on which treatment was administered. Error bars=SEM. n=15 in each group (pooled data from 2 experiments). Panel B: Final tumor volumes, n=15 per treatment. Panel C: Final weights for each tumor, with the bars representing the mean values for each group (n=15). Panel D: Spaghetti plots to show individual tumor growth curves (tumor volume) for the duration of the experiment. Fractions displayed in each graph represent the number of mice (out of 15 animals) with no detectable tumor at the time of sacrifice. Statistical analyses were performed by Brown-Forsythe ANOVA and Dunnett's T3 multiple comparisons test, *p<0.05, **p<0.01, ***p<0.001, ****p<0.0001, NS=not significant.

FIG. 6, panels A-B, shows flow cytometric analysis of tumor-infiltrating CD8+ T-cells: Panel A: activated cytotoxic T cells, determined by CD107a and GzmB expression; Panel B: PD-1 staining intensity. Group sizes: n=10 for saline and SB415286 treatments, n=9 for free AZD1080 or sAZD1080 treatment, and n=3 for anti-PD-1 (the reduced animal number for the latter group was due to disappearance or extremely reduced size of most of the tumors in this group). Statistical analysis was performed by Brown-Forsythe ANOVA and Dunnett's T3 multiple comparisons test. *p<0.05, **p<0.01, ***p<0.001, ****p<0.0001, NS=not significant.

FIG. 7, panels A-D, shows immunohistochemical analysis of MC38 subcutaneous tumors. Panel A: Representative images of tumor sections stained with anti-CD8, anti-perforin, or anti-cleaved-caspase 3 antibodies. Scale bar (lower right corner)=100 μm; all images were reviewed under the same magnification. Quantitative image analysis showing: Panel B: CD8⁺ cells per mm², (panel C) perforin⁺ cells per mm², and (panel D) cleaved caspase 3⁺ (CC3) cells per mm². The bars represent the mean values for each group. Statistical analysis by 2-way Brown-Forsythe ANOVA and Dunnett's T3 multiple comparisons test, n=6 for saline and free AZD1080 treatments, 5 for silicasome AZD1080 and anti-PD-1 treatments, and 8 for SB415286 treatment. *p<0.05, **p<0.01.

FIG. 8, panels A-C, illustrates tumor volume growth inhibition by PD-1/PD-L1 axis inhibition in three additional syngeneic tumor models in mice. Tumor volume growth inhibition by treatment with anti-PD-1 antibody or silicasome AZD1080 for panel A) CT colon cancer model, (panel B) LLC lung cancer model, and (panel C) KPC pancreatic cancer model. Animal treatment is discussed in the method section. For each model, tumor volume growth curves are shown starting from time of inoculation to time of sacrifice. Green arrows represent days of treatment administration. Error bars=SEM. The spaghetti plots are used to show the growth curve for every animal, in addition to blocking of the final tumor volumes for each model, with the bars representing the mean values for each group. Statistical analysis by Brown-Forsythe ANOVA and Dunnett's T3 multiple comparisons test. For CT26, n=9 for saline treatment and n=6 for silicasome AZD1080 and anti-PD-1 treatments. For LLC, n=9 for saline and 7 for silicasome AZD1080 and anti-PD-1 treatments. For KPC, n=10 for saline, 7 for silicasome AZD1080, and 8 for anti-PD-1 treatments. *p<0.05.

FIG. 9, panels A-B, shows a histological comparison of the immune responses across the syngeneic cancer models. Immunohistochemistry was performed using anti-CD8 or anti-cleaved caspase 3 antibodies on tumor sections from mice treated with saline, silicasome AZD1080 (sAZD1080) or anti-PD-1 antibody (aPD1). Quantification of positively stained cells was performed for (panel A) anti-CD8+ cells per mm2, or (panel B) anti-cleaved caspase 3+ cells per mm2. The bars represent the mean value for each group. The group sizes for CT26 were: n=9 for saline treatment, n=6 for sAZD1080, as well as anti-PD-1. Group sizes for LLC: n=9 for saline, n=7 for sAZD1080, as well as for anti-PD-1 treatments. Group sizes for KPC: n=10 for saline, n=7 for sAZD1080, and n=8 for anti-PD-1. The MC38 data were derived from the experiment shown in FIG. 7. N values were higher for saline treatment groups due to previously recognized increased variance in tumor sizes. Statistical analyses were performed by 2-way Brown-Forsythe ANOVA and Dunnett's T3 multiple comparisons test. *p<0.05, **p<0.01 compared to saline control, and †p<0.05 compared to anti-PD-1 treatment.

FIG. 10 illustrate the structures of 17 commercially available GSK3 inhibitors. Red boxes indicate compounds not experimentally included in this study due to chemical properties that are conducive to remote loading. Yellow boxes indicate compounds selected for initial study but ruled out experimentally. The green box indicates the compound selected for further study due to chemical properties and experimental validation. The blue box indicates compound included in further studies as a positive control.

FIG. 11 illustrates encapsulation efficiency of GSK3 inhibitors in silicasomes. Encapsulation of AZD1080, AZD2858, LY2090314, or 1-azakenpaullone by remote loading at 5, 10, or 20 feed weight % (relative to silicasome weight). AZD2858 precipitated at 20 wt % and encapsulation efficiency could not be assessed. n=4 independent loading experiments, error bars denote SD. Comments on loading efficiency are provided beneath each graph.

FIG. 12 shows silicasome morphology prior to performance of AZD1080 remote loading. CryoTEM and corresponding DLS and zeta potential data. Scale bar=100 nm, reported error is SD., n=3.

FIG. 13 illustrates cumulative AZD1080 release from silicasomes over time. AZD1080-laden silicasomes were incubated in 100% FBS for 24 hours at 37° C. and were sampled periodically to assess cumulative drug release. N=4, error bars denote SD.

FIG. 14 shows mouse body weight assessment in response to one-time injection of free AZD1080 at various concentrations. Dotted lines represent thresholds for toxicity (−15%) and weight loss requiring euthanasia (20%). Body weight changes are relative to individual mouse initial weights. N=4 mice per group, error bars are SEM. No significant differences detected by 2-way ANOVA.

FIG. 15 shows final tumor volumes for each treatment group, during the performance of two sets of experiments in the MC38. Tumor model. Box and whisker plots shown, n=6 mice per treatment for experiment 1 and n=9 mice per treatment in experiment 2. No statistical significance was observed between comparable groups in experiments 1 and 2. F(1,65)=0.02995; p=0.8631. Statistical significance was determined by 2-way ANOVA.

FIG. 16 shows low magnification images of the immunohistochemistry staining of MC38 subcutaneous tumors. All images are displayed at 10× magnification and are representative of overall staining. All images represent the same magnification.

FIG. 17 shows tumor weight in the MC38 model over the course of the treatment observation period. Arrows point to the time of treatment administration. N=15 mice per group, error bars are SEM. No significant differences detected by 2-way ANOVA.

FIG. 18, panels A-B, shows blood biochemistry in the MC38 mouse model. Panel A: Blood chemistry panel. N=9, all data points shown on graph, horizontal bars represent mean values per treatment group. Significance determined by 1-way Brown-Forsythe ANOVA test and Dunnett's T3 multiple comparisons test. Where p values are not shown, the comparison was not significant p>0.05. Panel B: Principle component analysis of blood chemistry variables. Dotted ellipses represent 95% confidence intervals.

FIG. 19 shows MC38 mouse organ H&E histology. Representative micrographs of the liver, kidney, spleen, and lung from MC38-laden mice treated with saline, sAZD1080, aPD1, free AZD1080, or SB415286. All images are presented at the same scale.

### DETAILED DESCRIPTION

Described herein are nanoparticle drug delivery vehicles that effectively deliver inhibitors of glycogen synthase kinase 3 (GSK3) to target cells, e.g., to cancer cells. The GSK3 inhibitors can inhibit the expression of PD-1 in the target cells and can thereby provide an effective modality in the treatment of various cancers. In certain embodiments the drug delivery vehicles comprise a nanoparticle comprising one or more cavities disposed within the nanoparticle and having an outside surface where the one or more cavities are in fluid communication with the outside surface, a GSK3 inhibitor disposed within the one or more cavities; and a lipid bilayer disposed on the surface of the nanoparticle where said lipid bilayer fully encapsulates the nanoparticle.

In certain embodiments other embodiments, the drug delivery vehicles comprises solid nanoparticles and the GSK3 inhibitor(s) are disposed within the nanoparticle material (e.g., intermixed with the nanoparticle material or adsorbed to, or covalently or ionically bound to, the nanoparticle surface) and a lipid bilayer disposed on the surface of the nanoparticle where said lipid bilayer fully encapsulates the nanoparticle.

In certain embodiments the drug delivery comprises a liposome and the GSK3 inhibitor is disposed within the liposome.

Additionally, methods of use of the drug delivery vehicles are described in certain embodiments such method of use include, but are not limited to the treatment of various cancers.

### GSK3 Inhibitors.

In various embodiments the drug delivery nanoparticles described herein contain one or more GS3K inhibitors. GSK3, is a signaling hub protein at the intersection of several important intracellular signaling pathways, including in the post-TCR signaling cascade, is a well-established drug target for which over 20 compounds have been developed to inhibit this serine-threonine kinase (11, 12). More specifically, the use of the compound SB415286 to inhibit GSK3 was found to increase the expression of T-bet, a master transcriptional regulator in T-cells that have the capability of interfering in PD-1 expression (10, 13, 14). Interference in PD-1 expression was shown to reverse the inhibition of cytotoxic T-cell responses in the mouse B16 melanoma cancer model, with an equal effectiveness as anti-PD-1 antibodies (14). Thus, small molecule inhibitors (SMI) of GSK3 offer the potential of being used as a surrogate or to augment the effect of immune checkpoint blocking antibodies, in addition to emerging use applications for Alzheimer's disease, diabetes and certain cancers.

We first sought to rationally select drug candidates to encapsulate within the nanoparticle drug carriers, as described herein. There are a number of mechanisms by which the drug carriers (e.g., silicasomes) can be loaded with compounds, but one of the most efficient methods is by remote-loading. Remote-loading, originally developed for the loading of doxorubicin into liposomes (e.g., to develop Doxil) is effective for loading weak bases into silicasomes or other nanoparticle carriers surrounded by a lipid bilayer. A survey of manufacturers and the literature produced a list of compounds that demonstrate selective GSK3 inhibitory activity as shown in Table 6 (in Example 1). This class includes a non-encapsulated SB415286 compound, which has been used in previous studies to demonstrate the relationship between PD1 expression and GSK3 inhibition.

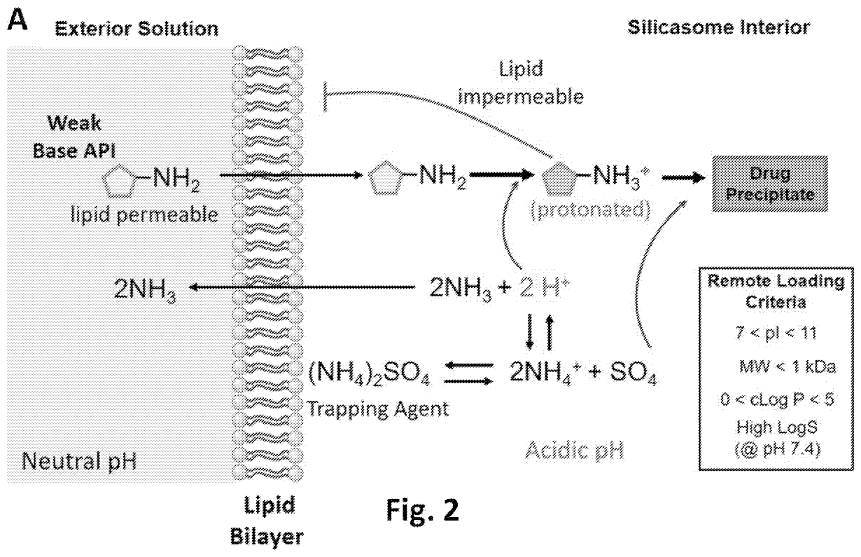

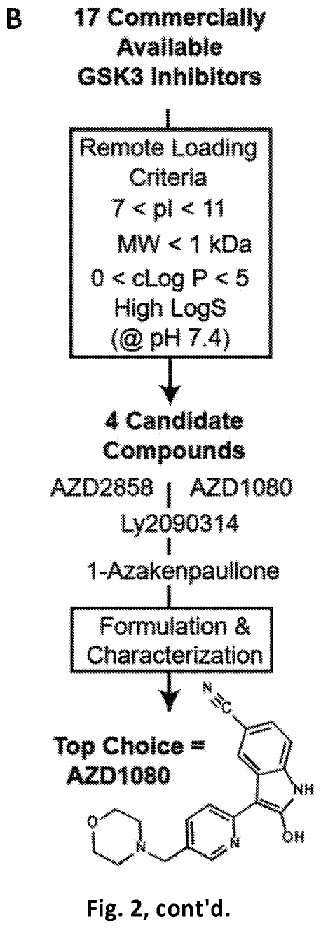

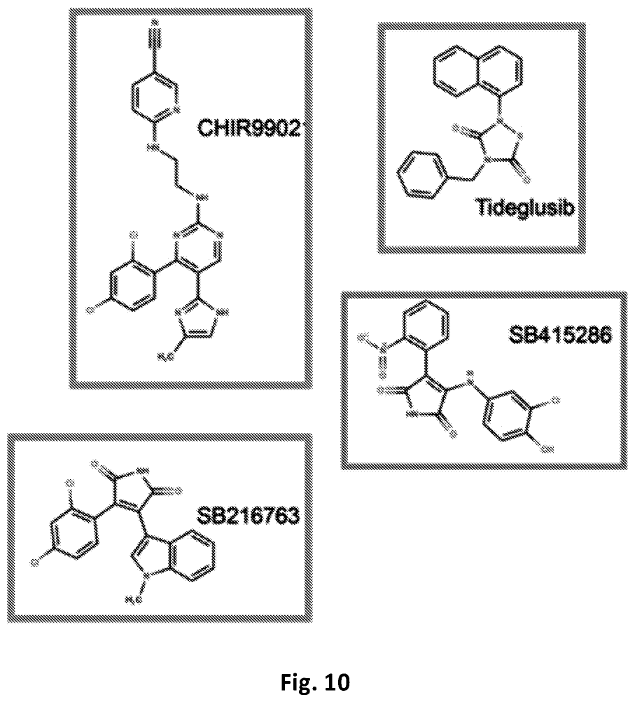

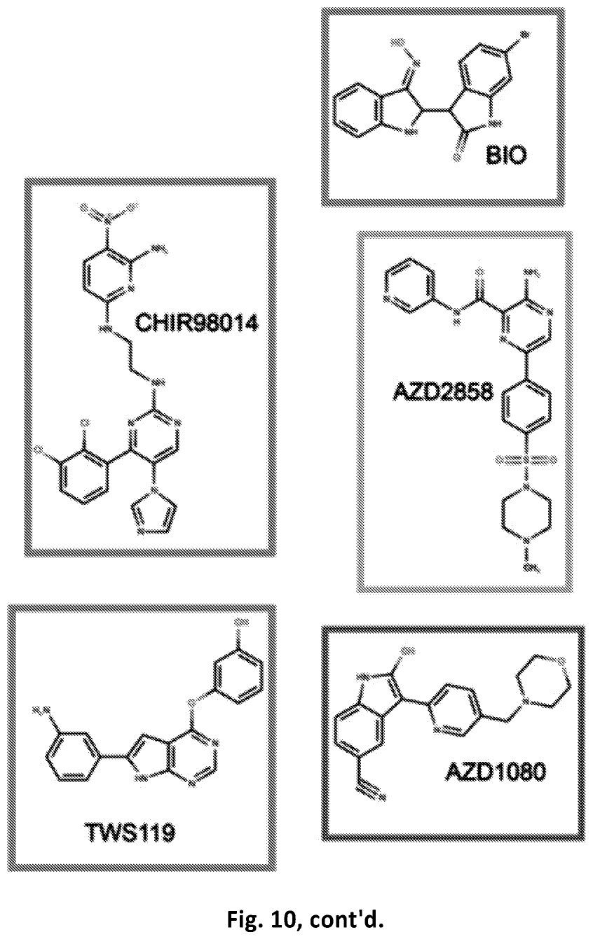

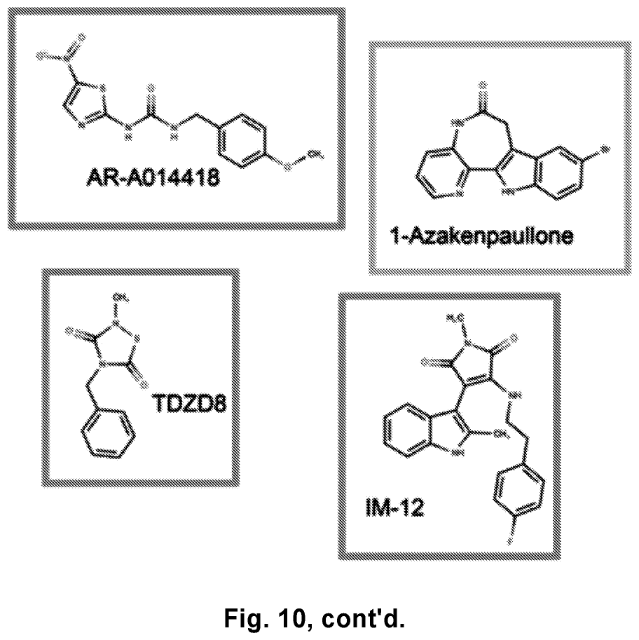

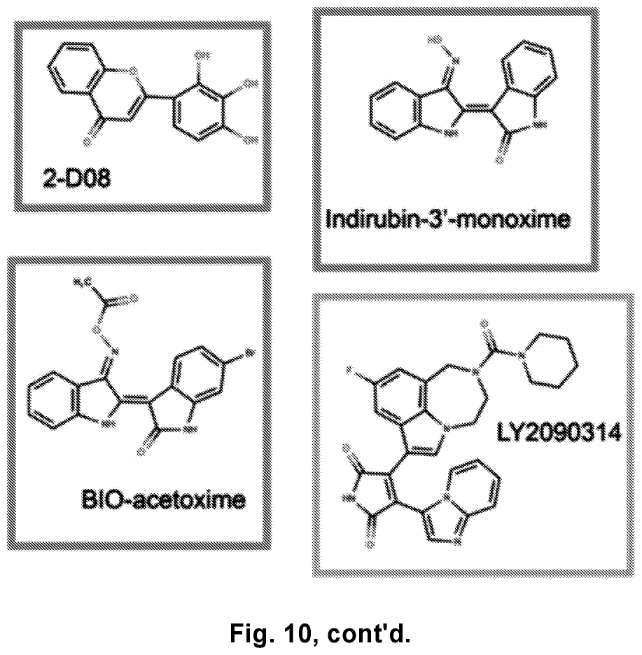

Of these 17 compounds, we collected published data and/or computationally derived data on the efficacy (IC50), hydrophobicity (cLogP), molecular weight, isoelectric point, and solubility (LogS) (FIGS. 2 and 10). These parameters were considered to be proxies for drug characteristics that allow remote control loading into nanocarriers, namely compounds that are weak bases, amphipathic, small enough to traverse the lipid bilayer, and soluble enough to prevent aggregation or coming out of solution. Considering these parameters, 7/17 compounds could be excluded from further consideration as they did not exhibit weak base characteristics. Of the remaining 10 materials, 4 (AZD2858, AZD1080, LY2090314, and 1-Azakenpaullone) showed aqueous solubility characteristics of low cLogP values and high logS values that were considered promising to attempt remote-loading. Of these four candidates, one compound (AZD1080) demonstrated the highest encapsulation efficiency and loading capacity (FIG. 2).

While AZD1080 was used as proof of principle (see, e.g., Example 1) it will be recognized that, in various embodiments, other GS3K inhibitors capable of remote loading (e.g., weakly basic GS3K inhibitors), including, but not limited to AZD2858, LY2090314, and 1-Azakenpaullone can readily be used in combination with AZD1080, or as an alternative to AZD1080.

In this regard, it is noted that tricyclic compounds that are GSK3 inhibitors are described in U.S. Patent Pub. No: US 2020/0109154 A1, substituted pyridines that are GSK3 inhibitors are described in U.S. Patent Pub. No: US 2011/0190316 A1, benzofuran-3-yl(indol-3-yl) maleimides that are GSK3 inhibitors are described in U.S. Patent Pub. No: US 2010/0004308 A1, and heteroaryl amines that are GSK3 inhibitors are described in U.S. Patent Pub. No: US 2005/0004125 A1 all of which are incorporated herein by reference for the GSK3 inhibitors described therein.

### Nanoparticles.

In various embodiments the drug delivery vehicles described herein comprise a nanoparticle containing one or more cavities where the nanoparticle is disposed within and fully encapsulated by a lipid bilayer. These nanoparticles include, but are not limited to a porous inorganic nanoparticle, a porous organic nanoparticle, or a metal-organic framework nanoparticle In certain embodiments the nanoparticle comprises a solid nanoparticle disposed within and fully encapsulated by a lipid bilayer where the cargo (e.g., GSK3 inhibitor) is adsorbed or covalently- or ionically bound to the surface of the nanoparticle. In certain embodiments the drug delivery vehicle comprise a liposome without a nanoparticle core.

Nanoparticle Comprising One or More Cavities.

Illustrative porous nanoparticles (e.g., nanoparticles containing one or more cavities) that can be coated with a lipid bilayer are shown in Table 1.

TABLE 1
Illustrative, but non-limiting examples of nanoparticles comprising one
or a plurality of cavities that can be coated with a lipid bilayer (LB).

| Nanoparticle | Inner Composition | Lipid Composition | Reference |
|---|---|---|---|
| Calcium Phosphate | Calcium phosphate | DOPC or | Li, Yang, and |
| siRNA | | DOTAP:Cholesterol:DSPE- | Huang JCR 2012, |
| Nanoparticles | | PEG 1:1:0.3 | 158, 1, 108-114 |
| Lipobeads | Poly(N- | Soy or Egg PC, | Rahni and Kazakov |
| | isopropylacrylamide) | Cholesterol | Gels 2017, 3, 1, 7 |
| | micro/nanogels | | |
| MOF@Lipid | Mesoporous Iron(III) or | DOPC or | Wuttke et al Chem |
| Nanoparticles | Mesoporous | DOTAP:Cholesterol:DSPE- | Commun 2015, 51, |
| | Chromium(III) | PEG 1:1:0.3 | 15752-15755 |
| | Carboxylate | | |
| Silicasomes | Mesoporous Silica | DSPE:Cholesterol:DSPE- | Meng et al ACS |
| | | PEG 6:4:0.3 | Nano 2015, 9, 4, |
| | | | 3540-3557 |
| Lipid Coated Silica | Hollow spherical silica | DOPC or bis- | Senarath-Yapa et al |
| Nanoparticles | nanoparticle | SorbPC | Langmuir 2007, 23, |
| | | | 25, 12624-12633 |
| Lipid-Coated | Hollow core Fe₃O₄ | DDAC | Katagiri, |
| Magnetic | | | Nakamura, and |
| Nanoparticles | | | Koumoto ACS |
| | | | Appl Mater |
| | | | Interfaces 2010, 2, |
| | | | 3, 768-773 |
| Magnetic | Core-Shell Fe₃O₄—SiO₂ | Custom Cationic | Mattingly et al |
| Nanoparticle- | Nanoparticles | Quaternary Amine | Langmuir 2015, 31, |
| Supported Lipid | | Lipid | 11, 3326-3332 |
| Bilayers | | | |
| Lipid Coated | Calcium carbonate | DOTAP, | Kim, Fotte, and |
| Calcium Carbonate | nanoparticle | Cholesterol, DSPE- | Huang Cancer |
| Nanoparticles | | PEG | Letters 2013, 334, |
| | | | 2, 311-318 |
| Lipid-Coated | Cisplatin Aggregate | DOTAP:Cholesterol:DSPE- | Guo et al ACS |
| Cisplatin | Nanoparticle | PEG 1:1:0.5 | Nano 2013, 7, 11, |
| Nanoparticles | | | 9896-9904 |
| Bilayer-Coated | Polyacrylamide N,N- | DOPC:Egg PG:Cholesterol | Schillemans et al |
| Nanogels | methylenebisacrylamide | 4:1:1 | Macromolecules |
| | nanogel | | 2006, 39, 17, 5885- |
| | | | 5890 |
| Lipid-Bilayer | Mesoporous carbon | DSPE-PEG:Soy PC:DPPC | Li et al ACS Appl |
| Coated Mesoporous | nanoparticle | 10:5:63 | Mater Interfaces |
| Carbon | | | 2018, 10, 23, |
| Nanoparticles | | | 19386-19397 |

It will be noted that in certain embodiments, any of the nanoparticles shown in Table 1, can additionally or alternatively have a cargo disposed on the surface of the particle, e.g., through adsorption, ionic binding, or covalent linkage (e.g., direct or through a linker).

Porous Inorganic Nanoparticles

Porous inorganic nanoparticles include, but are not limited to porous calcium carbonate nanoparticles, porous calcium phosphate nanoparticles and porous silica nanoparticles. In certain embodiments the porous inorganic nanoparticle comprises a single cavity. In certain embodiments this cavity is simply a channel into the nanoparticles. In certain embodiments the cavity comprises a hollow interior of the nanoparticle with a single aperture (pore) penetrating through to the surface of the nanoparticle. In certain embodiments the single-cavity nanoparticle comprises a nanobowl (i.e., a bowel shaped nanoparticle (concave nanostructures with an opening)). Single cavity nanoparticle may be fabricated by any of a number of methods well known to those of skill in the art. By way of example, lipid bilayer covered nanobowls are described by Chen et al. (2020) Nano Letters, DOI: 10.1021/acs.nanolett.0c00495.

In certain embodiments the porous nanoparticle comprise a porous silica nanoparticle. In certain embodiments the porous silica nanoparticle comprises a mesoporous silica nanoparticle (MSN), a mesoporous organosilica nanoparticle (MON), and/or a periodic mesoporous organosilica (PMO) nanoparticle.

MSNs, MONs, and PMOs are commonly fabricated using sol-gel processes in aqueous solutions (Croissant et al. (2015) Nanoscale, 7: 20318-20334; Wu et al. (2013) Chem. Soc. Rev. 42: 3862-3875; Yano & Fukushima (2004) J. Mater. Chem. 14: 1579-1584; Nakamura et al. (2007) J. Phys. Chem. C, 111: 1093-1100). The conventional sol-gel synthesis has been studied extensively and allows precise control of nanoparticle properties such as size, pore size and geometry, particle modification, and/or surface functionalization (see, e.g., Wu et al. (2013) Chem. Soc. Rev. 42: 3862-3875). In one illustrative sol-gel synthesis, silica particles are formed via hydrolysis of various silanes and/or silicates with a subsequent silica condensation:

—Si—O—+HO—Si—→—Si—O—Si—+OH—

In one illustrative, but non-limiting embodiment, synthesis takes place in an aqueous solution and can involve alcohol and ammonia or other catalysts (see, e.g., Yano & Fukushima (2004) J. Mater. Chem. 14: 1579-1584). The speed of the synthesis reaction depends on the pH value with the maximum silica condensation rate at normal pH conditions. The types and concentrations of the synthesis reagents affect the resulting particle size. Tetraethyl orthosilicate (TEOS), tetramethyl orthosilicate (TMOS) and other compounds can be used as silicon sources. To inhibit silica growth and, thus, obtain smaller MSNs, surface-protection agents can be used, such as triethanolamine (ILA), poly (ethylene glycol) (PEG) and/or a second nonionic surfactant (see, e.g., Möller et al. (2007) Adv. Funct. Mater. 17: 605-612). These agents can also be useful for isolation of the growing silica particles from each other, preventing their aggregation and the growth of silica bridges between neighboring particles.

In certain embodiments, to obtain MSNs, micelles can be used as a soft template to form the mesoporous structure. In one illustrative, but non-limiting embodiment, the silica particles are grown on the templates as starting points for the condensation. Surfactants such as cetyltrimethylammonium bromide (CTAB) can be added to the solution as well. At low concentrations just above the critical micellar concentration, the surfactant molecules bind together and form small spherical micelles. At higher concentrations, micelles can have cylindrical or other shapes. These micelles are positively charged and attract negatively charged silanes, facilitating their condensation. Addition of the second surfactant can lead to the formation of the more complicated micellar structures, allowing further modification of the MSNs pore structure. Similar to the micelles, vesicles can be used as templates for the MSN growth (see, e.g., Yeh et al. (2006) Langmuir, 22: 6-9). In certain embodiments, inorganic nanoparticles, such as metal (Au, Pt) or metal oxide (Fe₃O₄) nanoparticles could be incorporated into the structure of MSNs as desired (see, e.g., Kneževi′ et al. (2013) RSC Adv. 3: 9584-9593; Timin et al. (2016)Mater. Chem. Phys. 183: 422-429; Ott et al. (2015) Chem. Mater. 2015, 27: 1929-1942). They can be used as the templates for the MSNs growth as well. Such “hybrid” nanoparticles can be capable of both carrying a drug load and acting as contrast agents for bioimaging. In certain embodiments to produce larger pore sizes to accommodate higher quantities of molecules or simply larger molecules (e.g., biomolecules, such as DNA and proteins a swelling agent can be utilized. Several swelling agents can be used to increase the pore sizes, e.g., trimethylbenzene (TMB) (see, e.g., Zhang et al. (2011) J. Colloid Interface Sci. 361: 16-24). Another way to increase the size of the pores is the use of the block-polymers as templates (see, e.g., Han & Ying (2005) Angew. Chem. 117: 292-296).

In certain embodiments a so-called “liquid calcination” method using high boiling solvents can be used to retain liquid phase during calcination. Silanol groups can also be removed from MSN surface via a silane ethanol solution, reducing the bridging. The calcination process can be avoided entirely if templating is done using a thermosensitive polymer (poly (N-isopropylacrylamide)), which forms aggregates at higher temperatures and dissolves at lower temperatures (see, e.g., Du et al. (2009) Langmuir, 25: 12367-12373).

The mixture of silane [usually tetraethyl orthosilicate(TEOS)] and an organosilane induces the formation of MONs and PMO. In this case, in certain embodiments, the surfactant templates can be removed with less aggressive extraction procedures, in order not to destroy the inorganic-organic framework of MONs and PMO. In certain embodiments, the calcination procedure, which can be used for MSNs, may not be completely appropriate for MONs and PMO. In general, harsh pH and temperature conditions are usually employed for the extracting process. The silica-etching chemistry [alkaline or hydrofluoric acid(HF) etching] can be introduced into the synthesis to form the hollow PMO structure (see e.g., Chen et al. (2013) Adv. Mater. 25: 3100-3105). For this, the PMO layer can be directly deposited onto the surface of silica particles in order to form well-defined solid silica core/PMO shell.

The chemical stability of some families of PMOs is higher than for the silica particles under etching. Therefore, the silica core can be selectively removed under alkaline or HF etching conditions, producing hollow periodic mesoporous structure. Illustrative, but non-liming examples of fabrication protocols are described by Wu et al. (2013) Chem. Soc. Rev. 42: 3862-3875 and by Chen et al. (2014) J. Am. Chem. Soc. 136: 16326-16334.

Uniform mesoporous silica particles of different diameters can be prepared using various synthetic conditions (e.g., controlling pH values or time of reaction). For instance, a simple method for tailoring the size of well-ordered and dispersed MSNs by adjusting the pH of the reaction medium, which leads to the series of MSNs with diameter sizes ranging from 30 to 280 nm is described by Lu et al. (2009) Small, 5: 1408-1413. It also possible to control particle growth at different times of the reaction. Smaller particles (140 nm) emerged for 160 s into the reaction process grew to their final size (500 nm) in 600s.

In one illustrative, but non-limiting embodiment, mesoporous silica nanoparticles (MSNPs) are synthesized as a large batch, as previously described by Liu et al. (2019) ACS Nano. 13(1): 38-53. As illustrated herein in Example 1, this involves the addition of 0.9 L of 25 wt % CTAC in water to 17.1 L pure water in a beaker, stirred at 85° C. 72 g triethanolamine is added, followed by 600 mL TEOS. After stirring for 4 hours and cooling to room temperature, the bare MSNPs are precipitated with ethanol and CTAC is removed by washing in acidic ethanol, with sonication. MSNPs at 80 mg/mL in ethanol are centrifuged at 21,000×g for 15 minutes to pellet the nanoparticles. After removal of the ethanol supernatant, the MSNP pellet is resuspended in 123 mM ammonium sulfate in water by bath sonication.

Potential bioaccumulation is one of the biggest limitations for silica nanodrug delivery systems in cancer. Accordingly, in certain embodiments, the porous silica nanoparticles described herein (e.g., mesoporous silica nanoparticles) are modified to improve degradation and clearance. In one illustrative, but non-limiting example, the nanoparticles comprise a mesoporous silica/hydroxyapatite (MSNs/HAP) hybrid drug carrier, that provides enhanced biodegradability of silica. Synthesis of such nanoparticles is described by Hao et al. (2015) ACS Nano, 9(10): 9614-9625.

Other approaches for improving silica nanoparticle degradation include, but are not limited to noncovalent organic doping of silica, covalent incorporation of either hydrolytically stable or redox- and enzymatically cleavable silsesquioxanes, as well as bridged silsesquioxane (BS), and periodic mesoporous organosilica (PMO) NPs. Inorganically doped silica particles such as calcium-, iron-, manganese-, and zirconium-doped NPs, can also be used (see, e.g., Croissant et al. (2017) Adv. Mater., 29: 1604634).

In certain embodiments the mesoporous silica nanoparticles can be imine-doped silica nanoparticles. These nanoparticles contain imine groups embedded within the silica framework (see, e.g., Travaglini et al. (2019) Mater. Chem. Front., 3: 111-119). These methods of increasing degradability of silica nanoparticles are illustrative and non-limiting. Using the teaching provided herein, numerous other porous silica nanoparticles modified for enhanced biodegradation will be available to one of skill in the art.

Illustrative mesoporous silica nanoparticles include, but are not limited to MCM-41, MCM-48, and SBA-15 (see, e.g., Katiyar et al. (2006) J. Chromatog. 1122(1-2): 13-20).

In certain embodiments the porous inorganic nanoparticle comprises a porous calcium carbonate nanoparticle or a porous calcium phosphate nanoparticle. Means for fabricating calcium carbonate or calcium phosphate nanoparticles are known to those of skill in the art (see, e.g., Oiso & Yamanaka (2018) Adv. Powder Technol., 29(3): 606-610; Trofimov et al. (2108) Pharmaceutics, 10: 167).

Methods for obtaining porous CaCO₃ nanoparticles include, but are not limited to chemical methods (see, e.g., Trushina (2014) Mater. Sci. Eng. C, 45: 644-658; Svenskaya et al. (2016) Adv. Powder Technol. 27: 618-624; Parakhonskiy et al. (2015) J. Nanobiotechnol. 13: 53. doi: 10.1186/s12951-015-0111-7; Salomao et al. (2017) Adv. Tissue Eng. Regen. Med. eISSN: 2572-8490), microbiological methods (see, e.g., Wang et al. (2010) J. Phys. Chem. B, 114: 5301-5308; Achal & Pan (2014) Appl. Biochem. Biotechnol. 173: 307-317; Chekroun et al. (2004) J. Sediment. Res. 74: 868-876; Rodriguez-Navarro et al. (2007) Geochim. Cosmochim. Acta. 71: 1197-1213). The latter approach involves a bacteria-mediated synthesis, employing products of microbial metabolism containing carbonate ions that react with the calcium ions present in the environment to form CaCO₃ (see, e.g., Salomão et al. (2017) Adv. Tissue Eng. Regen. Med. eISSN: 2572-8490). Chemical methods generally utilize an emulsion technique (see, e.g., Fujiwara et al. (2010) Cryst. Growth Des. 10: 4030-4037; Maleki Dizaj et al. (2015) Expert Opin. Drug Deliv. 12: 1649-1660) and a precipitation reaction (see, e.g., Shirsath et al. (2017) Ultrason. Sonochem. 35: 124-133). One conventional approach for industrial production of CaCO₃ nanoparticles is precipitation by carbon dioxide bubbling through a calcium containing solution (gas diffusion method) (see, e.g., Shirsath et al. (2017) Ultrason. Sonochem. 35: 124-133; Shirsath et al. (2015) Ultrason. Sonochem. 24: 132-139). Other chemical methods include, but are not limited to a controlled double-jet precipitation technique (see, e.g., Som et al. (2016) Nanoscale, 8: 12639-12647; Jiang et al. (2009) Chem. Commun. 5853-5855; Stavek et al. (1990)Mater. Lett. 9: 90-95) and a solvothermal growth method (see, e.g., Li et al. (2002) J. Cryst. Growth, 236: 357-362).

Similarly, there are many different methods to produce porous calcium phosphate nanoparticles with different morphologies and sizes. One illustrative synthesis method of porous calcium phosphate nanosized materials involves mixing of water soluble salts of calcium and phosphate. With this technique, it is possible to control size, shape and crystallinity of particles by changing conditions of the precipitation reaction. For example, to obtain porous calcium phosphate spherical nanoparticles microwave assisted hydrothermal method is often used. This method can involve adenosine 5-triphosphate disodium salt (ATP) as the phosphorus source and stabilizer (see, e.g., Qi et al. (2013) Chemistry, 19: 981-987). As one illustrative, but non-limiting example, porous calcium phosphate particles with an average diameter of 260 nm have been synthesized by mixing calcium chloride dehydrate with ATP and further microwave treatment (Id.). The resulted nanoparticles showed good stability in aqueous solutions at different pH for more than 150 h. Alternatively, porous calcium phosphate nanoparticles with different morphology and crystallinity can be prepared by changing the ratio of precursors.

Porous calcium phosphate nanoparticles can also be synthesized by aging a mixture of calcium hydroxide and sodium triphosphate in the presence of hydrochloric acid (see, e.g., Kandori et al. (2010) J. Phys. Chem. C, 114: 6440-6445). To obtain differently shaped particles, the aging temperatures can be varied from, e.g., 100 to 150° C., as well as amount of precursors in the reaction (Id.). Additionally, hollow calcium phosphate nanoparticles can be obtained with a templating method (see, e.g., Ding et al. (2015) J. Mater. Chem. B, 3: 1823-1830). Different materials can be employed as templates. These include, for example, soybean lecithin (Id.), block copolymer micelles (see, e.g., Bastakoti et al. (2012) Chem. Commun. 48: 6532-6534). As one illustrative, but non-limiting example, an anionic triblock copolymer (poly (styrene-acrylic acid-ethylene glycol) has been used to form calcium phosphate hollow particles (see, e.g., Yeo et al. (2012) Ceram. Int. 38: 561-570). This type of template helps to overcome the problem of crystal overgrowth and allows the formation of nanosized hollow particles around 30 nm. Liposomes can also be used as templates to form hollow calcium phosphate particles (see, e.g., Yeo et al. (2012) Ceram. Int. 38: 561-570; Schmidt et al. (2004) Chem. Mater. 16: 4942-4947; Schmidt & Ostafin (2002) Adv. Mater. 14: 532-535). For example, 1,2-dioleoyl-sn-glycero-3 phosphate sodium salt (DOPA) and 1,2-dipalmitoyl-sn-glycero-3-phosphate sodium salt (DPPA) has been used as templates due to their negative charged head group, which can help the deposition of calcium and phosphate ions around the liposomes (see, e.g., Yeo et al. (2012) Ceram. Int. 38: 561-570). Resulting hollow nanoparticles were 64 and 104 nm, respectively (Id.). Polymer complexes can be also employed as templates to form hollow calcium phosphate particles (see, e.g., Zhang et al. (2009) Biomed. Mater. 4: 031002). Hollow calcium phosphate microspheres have been prepared using chitosan-polyacrylic acid (CS-PAA) as the template (Id.). The formation mechanism of the hollow structure was based on the electrostatic interactions between chitosan (CS) and poly acrylic acid (PAA). The size of CS-PAA spheres could be adjusted by changing the ratio and concentration of CS and PAA in the reaction (Id.).

The foregoing nanoparticles are illustrative and non-limiting. Using the teachings provided herein, numerous other porous inorganic nanoparticles will be available to one of skill in the art.

Porous Organic Nanoparticles.

In certain embodiments the nanoparticle comprise a porous organic material. Illustrative porous organic materials include, but are not limited to porous biocompatible polymers. Such porous biocompatible polymers are well known to those of skill in the art. Thus, for example, U.S. Pat. No. 10,549,014 describes the synthesis of porous polymers of the polyaryletherketone (PAEK) family (e.g., polyether ether ketone (PEEK), carbon reinforced PEEK, polyether ketone ketone (PEKK), PEKEKK (polyetherketoneetherketoneketone), polyaryletherketone (PAEK), polyetherketone (PEK), Polyetherketone Etherketone Ketone (PEKEKK), and the like), polycaprolactone (PCL), polylactic acid (PLA), polyglycolic acid (PGA), polyphenylene, self-reinforced polyphenylene, polyphenylsulphone, poly sulphone, polyethylene terephthalate (PET), polyethylene, polyurethane, or a mixture thereof, and the like. Other porous biocompatible polymers include, for example, polymers include oligocarbonatedimethacrylate (OCM-2) porous polymer (see, e.g., Yudin et al. (2020) Polymer, 192: 122302 doi.org/10.1016/j.polymer.2020.122302), carbonate- and phthalate-containing dimethacrylates (see, e.g., Kovylin et al. (2019) Chem. Select, 4(14): 4147-4155), and the like.

In certain embodiments porous nanoparticles comprising the drug delivering vehicles described herein are formed from hydrogels. In this regard it is noted that the term “lipobeads” has been used to name spherical bipartite structures made of a hydrogel core coated with a lipid bilayer (see, e.g., Rahni & Kazarov (2017) Gels, doi.org/10.3390/gels3010007). In certain embodiments the hydrogel comprising the “nanoparticle” can comprise a hydrogel formed from one or more materials selected from the group consisting of poly(N-isopropylacrylamide) (PNIPA), poly(N-isopropylacrylamide-co-1-vinylimidazole) (PNIPA-VI), poly(acrylamide) (PAAm), poly(acrylamide), poly(N,N-dimethylacrylamide), poly(N,N-diethylacrylamide), poly(1-vinylimidazole), poly(sodium acrylate), poly(sodium methacrylate), poly(2-hydroxyethylmethacrylate) (HEMA), poly(N,N-dimethylaminoethyl methacrylate) (DMAEMA), poly(N-[tris(hydroxymethyl)methyl]acrylamide), poly(1-(3-methacryloxy)propylsulfonic acid) (sodium salt), poly(allylamine), poly(N-acryloxysuccinimide), poly(N-vinylcaprolactam), poly(1-vinyl-2-pyrrolidone), poly(2-acrylamido-2-methyl-1-propanesulfonic acid) (sodium salt), poly((3-acrylamidopropyl) trimethylammonium chloride), poly(diallyldimethylammonium chloride), and the like.

Methods of forming lipobeads comprising these and other hydrogels are described in U.S. Patent Pubs: US 2003/0035842 A1, US 2010/0062054 A1, US 2013/0202667 A1, and the like as well as Rahni & Kazarov (2017) supra.

Using the teachings provided herein, nanoparticles comprising the porous polymers described above, and/or other porous polymers are readily available to those of skill in the art and, using the teaching described herein, can be used in the fabrication of the drug delivery vehicles described herein.

Metal-Organic Framework Nanoparticles

In certain embodiments the nanoparticle comprise a metal organic framework (MOF) nanoparticle. Metal organic frameworks (MOFs) are a class of compounds consisting of metal ions or clusters coordinated to organic ligands to form one-, two-, or three-dimensional structures. They are a subclass of coordination polymers, with the special feature that they are often porous.

In various embodiments the MOF comprises metal ions or metal clusters and organic molecule linkers. In certain embodiments any metal ion can be used for the preparation of a MOF. In certain embodiments, the metal ion is selected from the group consisting of Zn, Cu, Ni, AI, Co, Fe, Mn, Cr, Cd, Mg, Ca, Zr, Gd, Eu, Tb, and mixtures thereof. In other embodiments, the metal ion is selected from the group consisting of Zn, Cu, Fe, Gd, Al, Mg, and mixtures thereof. In one embodiment, the metal ion is Zn. In another embodiment, the metal ion is Fe. In yet another embodiment, the metal ion is Cu. In yet another embodiment, the metal ion is Al.

In certain embodiments the metal-organic framework (MOF) may be a transition metal-based metal-organic framework (MOF). For example, the metal-organic framework (MOF) may be a zinc-based metal-organic framework (MOF), a cobalt-based metal-organic framework (MOF), a zirconium-based metal-organic framework (MOF), a chromium-based metal-organic framework (MOF), or other transition metal-based metal-organic frameworks (MOF).

In certain embodiments, the MOF is selected from the group consisting of zeolitic imidazolate frameworks (ZIFs), Universitetet i Oslo (University of Oslo) (UiOs), and (Materials of Institut Lavoisier frameworks (MILs). In other embodiments, the MOF is selected from the group consisting of ZIF-8, ZIF-67, ZIF-90, Fe-BTC, HKUST-1, and MIL-53, MIL-89, MIL-88A, MIL-100, UiO-66, UiO-66-NH₂, MOF-801, MOF-804, Fe-NDC-M, and Fe-NDC-O MOFs. In certain embodiments the MOF is MIL-88A. In certain embodiments the MOF is ZIF-8. Methods of synthesizing such MOFs are described in U.S. Patent Publication Nos: US 2017/0232420 A1, and US 2019/0247502 A1 which are incorporated herein by reference for the MOFs and synthesis methods described therein.

Other suitable MOFs include but are not limited to MOF-1201 (Ca¹⁴(L-lactate)₂₀(Acetate)₈(C₂H₅OH)(H₂O)] and MOF-1203 [ Ca6(L-lactate)₃(Acetate)₉(H₂O)], based on Ca²⁺ ions and innocuous lactate and acetate linkers (see, e.g., U.S. Patent Pub. No: US 2020/0095264 A1 which is incorporated herein by reference for the MOFs and synthesis methods described therein.

The MOFs can be synthesized by any of a number of methods well known to those of skill in the art. By way of illustrative, but non-limiting example, In certain embodiments MOFs may be synthesized from a metal salts/metal ions and organic ligands. In some embodiments, the organic ligands may be any suitable mono-, di-, tri-, or tetravalent ligands. The metal salt/metal ion may be of any suitable metal, such as a transition metal, for example: Iron (Fe), Titanium (Ti), or zirconium (Zr). In some embodiments, MOFs, including those based on Fe-NDC-M and Fe-NDC-O, may be synthesized using iron nitrate nonahydrate (Fe(NO₃)₃9H₂O), and 2,6 naphthalenedicarboxylic acid (2,6-NDC). In some embodiments, iron nitrate nonahydrate and 2,6-NDC may be reacted in the molar ratio of 10-1:10, 5:1-1:5, 3:1-1:3. 2:1-1:2, 1.5:1-1:1.5, or 1:1. Iron nitrate nonahydrate and 2,6 NDC may be reacted in a solvent. In some embodiments, the solvent may be dimethylformamide (DMF), dimethylacetamide (DMAC) or dimethylsulfoxide (DMSO). Iron nitrate nonahydrate and 2,6 NDC may be stirred in the solvent.

Then the mixture may be reacted. In some embodiments, the mixture may be subject to microwave irradiation. For example, the mixture may be heated in a microwave oven. The mixture may be subject to microwave irradiation of about 10 W-500 W, 10 W-300 W, 10 W-300 W, 20 W-250 W, 30 W-250 W, 50 W-250 W, 100 W-200 W, or 150 W-200 W. The mixture may be irradiated for 30 sec or longer, 1 min or longer, 2 min or longer, 3 min or longer, 5 min or longer, or 10 min or longer.

In some embodiments, the mixture may be heated instead of or in addition to microwave irradiation. For example, the mixture may be heated in an oven, such as a conventional electrical oven. In some embodiments, the mixture may be heated at 50° C.-200° C., 50° C.-170° C., 70° C.-170° C., 70° C.-150° C., 70° C.-130° C., 80° C.-120° C., or 90° C.- 110° C. In some embodiments, the mixture may be heated for 3 hours or longer, 5 hour or longer, 10 hours or longer, 15 hours or longer, 20 hours or longer, or 24 hours or longer. The product may be separated from the reaction mixture, for example by centrifuge, washing and/or drying.

In one specific embodiment MIL-88A MOFs can be synthesized according to the protocol described by Illes et al. (2017) Chem. Mater. 29(19): 8042-8046. As described therein, MIL-88A MOFs are synthesized in a microwave assisted approach. In this synthesis route an aqueous solution of FeCl₃·6H₂O (1.084 g, 4.01 mmol) and fumaric acid (485 mg, 4.18 mmol) are given to water (20 ml, Milli-Q). The reaction mixture is stirred until the metal salt is completely dissolved. The reaction mixture was then given into a Teflon tube (80 ml) and placed into a microwave oven (e.g., Synthos 3000, Anton-Paar) along with 3 additional vessels. Two of these vessels are filled with water (20 ml), the third vessel is filled with an aqueous FeCl₃ (20 ml, 1.084 g, 4.01 mmol) and is used to monitor the reaction progress. The vessels were heated under stirring with the sequence comprising heating 30 seconds to 80° C., holding at 80° C. for 5 min, followed by cooling over 45 min to room temperature.

In another illustrative, but non-limiting embodiment, MOF hollow spheres with controlled size in the 35-2000 μm range including MIL-88A frameworks, as well as various functional nanoparticles (silica, cobalt, and UiO-66(Zr) MOF) can be synthesized by interfacial reaction using a continuous-flow droplet microfluidic system in a single step and one-flow strategy (see, e.g., Jeong et al. (2015) Chem. Mater. 27(23): 7903-7909.

It is also noted that lipid membrane coated MOFs are described in Cheng et al. (2018) J. Am. Chem. Soc. 140(23): 7282-7291 and Illes et al. (2017) Chem. Mater. 29(19): 8042-8046.

In various embodiments the nanoparticles comprising the drug delivery vehicle described herein can include particles as large (e.g., average or median diameter (or other characteristic dimension) as about 1000 nm. However, in various embodiments the nanoparticles are typically less than 500 nm or less than about 300 nm as, in general, particles larger than 300 nm may be less effective in entering living cells or blood vessel fenestrations. In certain embodiments the nanoparticles range in size from about 40 nm, or from about 50 nm, or from about 60 nm up to about 100 nm, or up to about 90 nm, or up to about 80 nm, or up to about 70 nm. In certain embodiments the nanoparticles range in size from about 60 nm to about 70 nm. Some embodiments include nanoparticles having an average maximum dimension between about 50 nm and about 1000 nm. Other embodiments include nanoparticles having an average maximum dimension between about 50 nm and about 500 nm. Other embodiments include nanoparticles having an average maximum dimension between about 50 nm and about 200 nm. In some embodiments, the average maximum dimension is greater than about 20 nm, greater than about 30 nm, greater than 40 nm, or greater than about 50 nm. Other embodiments include nanoparticles having an average maximum dimension less than about 500 nm, less than about 300 nm, less than about 200 nm, less than about 100 nm or less than about 75 nm. As used herein, the size of the nanoparticle refers to the average or median size of the primary particles, as measured by transmission electron microscopy (TEM) or similar visualization technique.

In certain embodiments the drug delivery vehicles have an average hydrodynamic diameter ranging from about 30 nm up to about 300 nm, or from about 30 up to about 200 nm, or from about 30 up to about 170 nm, or from about 30 nm up to about 150 nm, or from about 30 up to about 100 nm, or from about 30 up to about 80 nm, or from about 30 up to about 70 nm, or from about 40 up to about 70 nm by DLS. In certain embodiments the drug delivery vehicles have an average hydrodynamic diameter ranging from about 70 nm up to about 165 nm by DLS by DLS. In certain embodiments the nanoparticle comprising the drug delivery vehicle have an average pore size that ranges from about 1 to about 20 nm, or from about 1 to about 10 nm, or from about 1 to about 5 nm, or from about 1 to about 4 nm, or from about 1 to about 3 nm, or from about 2 to about 3 nm.

Illustrative mesoporous silica nanoparticles include, but are not limited to MCM-41, MCM-48, and SBA-15 (see, e.g., Katiyar et al. (2006) J. Chromatog. 1122(1-2): 13-20).

Using the teachings provided herein, nanoparticles comprising the metal organic framework nanoparticles described above, and/or other MOFs are readily available to those of skill in the art and, using the teaching described herein, can be used in the fabrication of the drug delivery vehicles described herein.

Solid Nanoparticles.

Additionally, a number of solid nanoparticles can readily be coated with a lipid bilayer and a cargo (e.g., GSK3 inhibitor) can be attached to the surface of the nanoparticle for delivery. In certain embodiments attachment to the nanoparticle surface is by adsorption, or ionic linkage, or covalent linkage (e.g., direct or through a linker). Illustrative, but non-limiting examples of such lipid bilayer (LB) encapsulated “solid” nanoparticles are shown in Table 2.

TABLE 2
Illustrative, but non-limiting examples of solid drug-delivery
nanoparticles that can be encapsulated by a lipid bilayer (LB).

| Nanoparticle | Inner Composition | Lipid Composition | Reference |
|---|---|---|---|
| Lipid-Coated | PLGA | DOPC:DOPG:DOPE:Sphingo- | Bershteyn et al. |
| Polymeric | | myelin:Cholesterol | (2008) Soft Matter, |
| Nanoparticles | | 1:1:2.2:2:5 | 4: 1787-1791 |
| Lipid coated PLGA | PLGA | Lipids or cell | |
| | | membrane | |
| Erythrocyte | PLGA | Red Blood Cell | Hu et al. (2011) |
| Membrane- | | Membrane | PNAS, 108(27): |
| Camouflaged | | | 10980-10985 |
| Polymeric | | | |
| Nanoparticles | | | |
| Hybrid Bilayer | Spherical gold | Soy PC, | Sitaula et al. (2008) |
| Coated Gold | nanoparticle | 1-decanethiol | Chem. Commun. |
| Nanoparticle | | | 3013-3015 |
| Light-Biovectors | Crosslinked | DSPC, Cholesterol | Fenart et al J Pharm |
| | maltodextrin | | Exp Thera 1999, |
| | polysaccharide | | 291, 3, 1017-1022 |
| Lipid-Coated | PLGA-Chitosan | DPPC | Schafer et al Adv |
| Biodegradable | Nanoparticles | | Sci Tech 2008, 57, |
| Nanoparticles | | | 148-153 |
| Lipid-Coated | Polystyrene | Egg PC:Choles- | Messerschmidt et al |
| Polymeric | nanoparticles | terol:DSPE-PEG | JCR 2009, 137, 1, |
| Nanoparticles | | 6.5:3:0.5 | 69-77 |
| Lipid Coated Silica | Spherical silica | DOPC or bis- | Senarath-Yapa et al |
| Nanoparticles | nanoparticle | SorbPC | Langmuir 2007, 23, |
| | | | 25, 12624-12633 |
| Lipid-Coated Silver | Spherical silver | POPC:POPG:Cholesterol | Bhowmik et al ACS |
| Nanoparticles | nanoparticle | 1:1:1 | Nano 2015, 9, 9, |
| | | | 9070-9077 |
| Lipid-Coated Gold | Gold Nanorods | DPPC:DPPG:Choles- | Kang and Ko Int J |
| Nanocomposites | | terol:DSPE-PEG | Nanomedicine |
| | | 6:1:3:0.3 | 2015, 10, 33-45 |
| Lipid-Coated | Hollow core Fe₃O₄ | DDAC | Katagiri, |
| Magnetic | | | Nakamura, and |
| Nanoparticles | | | Koumoto ACS |
| | | | Appl Mater |
| | | | Interfaces 2010, 2, |
| | | | 3, 768-773 |
| Liposome | Nucleic acid + | DOTAP:Cholesterol 1:1 | Li and Huang BBA |
| Polycation DNA | protamine | | Biomembranes |
| Nanoparticle | | | 2009, 1788, 10, |
| | | | 2259-2266 |
| Magnetic | Core-Shell Fe3O4—SiO2 | Custom Cationic | Mattingly et al |
| Nanoparticle- | Nanoparticles | Quaternary Amine | Langmuir 2015, 31, |
| Supported Lipid | | Lipid | 11, 3326-3332 |
| Bilayers | | | |
| Porphyrin- | Core-Shell | Porphyrin- | Rieffel et al Adv |
| Phospholipid- | NaYbF4:Tm—NaYF4 | Phospholipid, | Mater 2015, 27, 10, |
| Coated | Upconverting | DSPE-PEG | 1785-1790 |
| Upconversion | Nanoparticle | | |
| Nanoparticles | | | |
| Supported Lipid | LiYF4:Tm/Yb | DOPA, DOPC, | Rojas-Gutierrez et |
| Bilayer | Nanocrystal | Cholesterol, Oleate | al Particle 2016, 33, |
| Upconversion | | | 12, 865-870 |
| Nanoparticles | | | |
| Lipid-Coated Zinc | Zinc oxide nanoparticle | DOPC | Ancona et al |
| Oxide | | | Nanomaterials |
| Nanoparticles | | | 2018, 8, 3, 143 |
| Phospholipid | Aluminum hydroxide | DOPC | Wang et al ACS |
| Bilayer-Coated | nanoparticle | | Appl Mater |
| Aluminum | | | Interfaces 2015, 7, |
| Nanoparticles | | | 12, 6391-6396 |

As noted above in certain embodiments, the “solid” nanoparticle comprise a nanoparticle comprising one or more biocompatible polymers. Illustrative biocompatible polymers include, but are not limited to poly(lactic-co-glycolic acid) (PLGA), Poly(glycolic acid) (PGA), Poly(lactic acid) (PLA), Poly(caprolactone) (PCL), Poly(butylene succinate), Poly(trimethylene carbonate), Poly(p-dioxanone), Poly(butylene terephthalate), Poly(ester aminde) (HYBRANE®), polyurethane, Poly[(carboxyphenoxy) propane-sebacic acid], Poly[bis(hydroxyethyl) terephthalate-ethyl orthophosphorylate/terephthaloyl chloride], Poly(β-hydroxyalkanoate), Poly(hydroxybutyrate), and Poly(hydroxybutyrate-co-hydroxyvalerate).

Liposomes.

As noted above, in certain embodiments, the nanoparticle component of the drug delivery vehicle can be eliminated and the vehicle will then simply comprise a liposome. Liposomes are drug delivery vehicles that can be formulated with a wide variety of natural, synthetic, and modified lipid species to deliver drugs. Methods of making liposomes are well known to those of skill in the art. An illustrative, but non-limiting list of liposome formulations in clinical use is shown in Table 3.

TABLE 3
Illustrative liposome formulations.

| Lipid composition | Refence |
|---|---|
| HSPC, DSPG, cholesterol | Immordino et al. (2006) Int. J. Nanomed. 1(3): 297- |
| (2:0.8:1 molar ratio) | 315; Meunier et al. (1991) J. Antimicrob. |
| | Chemother. 28(Suppl B): 83-91 |
| DSPC and cholesterol (2:1 molar | Immordino et al. (2006) Int. J. Nanomed. 1(3): 297- |
| ratio) | 315; Rivera (2003) Oncologist. 8(Suppl 2): 3-9; |
| | Tomkinson et al. (2003) Leuk. Res. 27(11): 1039- |
| | 1050 |
| HSPC, cholesterol, and PEG 2000- | Immordino et al. (2006) Int. J. Nanomed. 1(3): 297- |
| DSPE (56:39:5 molar ratio) | 315; Park (2002) Breast Cancer Res. 4(3): 95-99; |
| | Hoarau et al. (2004) Pharm. Res. 21(10): 1783-1789 |
| DSPC, cholesterol, and PEG 2000- | Hong (2004) J. Chinese Oncol. Soc. 20(2): 10-21 |
| DSPE (56:39:5 molar ratio) | |
| EPC and cholesterol (55:45 molar | Immordino et al. (2006) Int. J. Nanomed. 1(3): 297- |
| ratio) | 315; Park (2002) Breast Cancer Res. 4(3): 95-99; |
| | Gardikis et al. (2010) Int. J. Pharm. 402(1-2): 231- |
| | 237 |
| EPG and DM PC (3:5 molar ratio) | Chowdhary et al. (2003) J. Pharm. Pharmaceut. Sci. |
| | 6(1): 13-19; Fahr et al. (2005) Eur J. Pharm. Sci. |
| | 26(3-4): 251-265 |
| Cholesterol, Triolein, DOPC, and | Immordino et al. (2006) Int. J. Nanomed. 1(3): 297- |
| DPPG (1 1:1:7:1 molar ratio) | 315 |
| Cholesterol, Triolein, DOPC, and | Patil & Burgess (2005) In: Burgess D J, editor. |
| DPPG (1 1:1:7:1 molar ratio) | Injectable Dispersed Systems: Formulation, |
| | Processing and Performance (Drugs and The |
| | Pharmaceutical Sciences Series). New York: Marcel |
| | Dekker; 249-303 |
| DOPC and DOPE | Usonis et al. (2003) Vaccine, 21(31): 4588-4592; |
| | D'Acremont et al.(2006) J. Travel Med. 13(2): 78-83 |
| DOPC and DOPE | Herzog et al. (2009) Vaccine. 27(33): 4381-4387 |
| DOPC, cholesterol, and cardiolipin | Immordino et al. (2006) Int. J. Nanomed. 1(3): 297- |
| (90:5:5 molar ratio) | 315; Zhang et al. (2005) Eur. J. Pharm. Biopharm. |
| | 59(1): 177-187 |
| DOPC, cholesterol, and cardiolipin in | Immordino et al. (2006) Int. J. Nanomed. 1(3): 297- |
| 90:5:5 molar ratio | 315; Cattaneo et al. (2010) J. Appl. Toxicol. 30(8): |
| | 730-744 |
| DOTAP, DOPC, and paclitaxel | Immordino et al. (2006) Int. J. Nanomed. 1(3): 297- |
| (50:47:3 molar ratio) | 315; Michaelis & Haas (2007) In: Gregoriadis G, |
| | editor. Liposome Technology, Volume III: |
| | Interactions of Liposomes with the Biological |
| | Milieu. London, UK: Informa Healthcare; 151-170; |
| | Eichhorn et al. (2006) Cancer Biol. Ther. 5(1): 89- |
| | 96; Robert et al. (2009) J. Pharm. Sci. 98(2): 411- |
| | 429 |
| DPPC and cholesterol | Li et al. (2008) J. Aerosol Med. Pulm. Drug Deliv. |
| | 21(3): 245-254; Mossalam et al. (2010) Ther. Deliv. |
| | 1(1): 169-193 |
| Cholesterol and egg sphingomyelin | Immordino et al. (2006) Int. J. Nanomed. 1(3): 297- |
| (45:55 molar ratio) | 315; Rodriguez et al. (2009) Cancer, 115(15): 3475- |
| | 3482; Krishna et al. (2001) J. Pharmacol. Exp. Ther. |
| | 298(3): 1206-1212 |
| DPPC, MSPC, and PEG 2000-DSPE | Yarmolenko et al. (2010) Int. J. Hyperthermia. |
| (90:10:4 molar ratio) | 26(5): 485-498; Dromi et al. (2007) Clin. Cancer |
| | Res. 13(9): 2722-2727 |
| DM PC and soybean oil | Immordino et al. (2006) Int. J. Nanomed. 1(3): 297- |
| | 315 |
| DMPC, DM PG, and cholesterol | Immordino et al. (2006) Int. J. Nanomed. 1(3): 297- |
| | 315 |
| DOPC, cholesterol, and cardiolipin | Immordino et al. (2006) Int. J. Nanomed. 1(3): 297- |
| | 315; Pal et al. (2005) Anticancer Res. 25(1A): 331- |
| | 341 |
| DMPC and DMPG | Dragovich et al. (2006) Cancer Chemother. |
| | Pharmacol. 58(6): 759-764 |
| Monophosphoryl lipid A, cholesterol, | Powell et al. (2008) Expert Rev. Respir. Med. |
| DMPG, and DPPC | 2(1): 37-45; Ohyanagi et al. (2011) Jpn. J. Clin. |
| | Oncol. 41(5): 718-722 |
| SHPC, cholesterol, and DSPE-PEG | Immordino et al. (2006) Int. J. Nanomed. 1(3): 297- |
| | 315 |
| SPC, DPPG, cholesterol, and mPEG | Immordino et al. (2006) Int. J. Nanomed. 1(3): 297- |
| 2000-DSPE | 315; Stathopoulos & Boulikas (2012) J. Drug Deliv. |
| | 2012: 581363 |
| DPSC and DSPE-PEG (95:5 molar | Immordino et al. (2006) Int. J. Nanomed. 1(3): 297- |
| ratio) | 315; Yu et al.(2007) Anticancer Res. 27(4B): 2541- |
| | 2545; Hwang et al. (2011) Jpn. J. Clin. Oncol. 41(5): |
| | 624-629 |
| HSPC and cholesterol (2:1 molar | Tomkinson et al. (2003) Leuk. Res. 27(11): 1039- |
| ratio) | 1050; Duffaud et al. (2004) Eur. J. Cancer. 40(18): |
| | 2748-2752 |
| Cholesterol and egg sphingomyelin | Immordino et al. (2006) Int. J. Nanomed. 1(3): 297- |
| (45:55 molar ratio) | 315; Semple et al. (2005) J. Pharm. Sci. 94(5): |
| | 1024-1038 |
| Cholesterol and egg sphingomyelin | Immordino et al. (2006) Int. J. Nanomed. 1(3): 297- |
| (45:55 molar ratio) | 315 |
| DSPC, DSPG, and Tween | Immordino et al. (2006) Int. J. Nanomed. 1(3): 297- |
| | 315 |

Abbreviations: DOTAP, 1,2-dioleoyl-3-trimethylammonium propane; DPPC, dipalmitoylphosphatidylcholine; DOPA, 1,2-Dioleoyl-sn-Glycero-3-Phosphate; MSPC, monostearoylphosphatidylcholine; DPPG, dipalmitoylphosphatidylglycerol; DSPC, distearoylphosphatidylcholine; HSPC, hydrogenated soy PC; DMPG, 1-a-dimyristoylphosphatidylglycerol; DMPC, 1-a-dimyristoylphosphatidylcholine; DOPC, 1,2-Dioleoyl-sn-glycero-3-phosphocholine; DOPE, dioleoyl phosphatidylethanolamine; DSPG, distearoylphosphatidylglycerol; PEG2000-DSPE, polyethylene glycol 2000-distearoylphosphatidylethanolamine.

It will be recognized that the above-identified liposome formulations are illustrative and non-limiting. It will be recognized that, in certain embodiments, the liposome formulations described above can also be utilized to formulate the lipid bilayer that surrounds a nanoparticle as described herein. Similarly, in various embodiments, any of the lipid bilayer formulations described below can be used in simple liposome formulations for delivery of a GSK3 inhibitor as described herein.

### Lipid Bilayer (LB)

The drug carrier nanoparticles described herein comprise a nanoparticle comprising one or more cavities, e.g., a porous nanoparticle such as a mesoporous silica nanoparticle (MSNP)), coated with a lipid bilayer. In certain embodiments the bilayer composition is optimized to provide a rapid and uniform particle coating, to provide colloidal and circulatory stability, and to provide effective cargo retention, while also permitting a desirable cargo release profile.

In certain embodiments the lipid bilayer comprises a combination of a phospholipid, and cholesterol, and in certain embodiments, a pegylated lipid (e.g., PE-PEG₂₀₀₀, DSPE-PEG₂₀₀₀), or a factionalized pegylated lipid (e.g., DSPE-PEG₂₀₀₀-maleimide) to facilitate conjugation with targeting moieties or other moieties including, for example, a drug.

In certain embodiments, to attach a surface LB coating, a coated lipid film procedure can be utilized in which nanoparticle (e.g., MSNP) suspensions are added to a large lipid film surface, coated on, e.g., a round-bottom flask. Using different lipid bilayer compositions, a series of experiments can be performed to find a composition and optimal lipid/particle ratio that provides rapid and uniform particle wrapping, coating and effective cargo retention and/or release upon sonication. It is believed that this lipid composition and wrapping cannot be achieved by liposomal fusion to the particle surface under low energy vortexing conditions.

In certain illustrative, but non-limiting embodiments the lipid bilayer can comprise: 1) one or more saturated fatty acids with C14-C20 carbon chain, such as phosphatidylethanolamine (PE), dimyristoylphosphatidylcholine (DMPC), dipalmitoylphosphatidylcholine (DPPC), distearoylphosphatidylcholine (DSPC), and diactylphosphatidylcholine (DAPC); and/or 2) One or more unsaturated fatty acids with a C14-C20 carbon chain, such as 1,2-dimyristoleoyl-sn-glycero-3-phosphocholine, 1,2-dipalmitoleoyl-sn-glycero-3-phosphocholine,1,2-dioleoyl-sn-glycero-3-phosphocholine (DOPC), 1,2-dieicosenoyl-sn-glycero-3-phosphocholine; and/or 3) natural lipids comprising a mixture of fatty acids with C12-C20 carbon chain, such as Egg PC, and Soy PC, sphingomyelin, and 4) cholesterol (CHOL) and/or a modified cholesterol (e.g., cholesterol hemisuccinate (CHEMS)) the like. It is noted that, in certain embodiments, in order to compensate a positive charge, it is possible to use cholesteryl hemisuccinate (CHEMS) that carries one negative charge at pH>6.5 in the formulation. These lipids are illustrative but non-limiting and numerous other lipids are known and can be incorporated into a lipid bilayer for formation of a drug delivery nanocarrier (e.g., a bilayer-coated nanoparticle).

In certain embodiments the drug carrier comprises bilayer comprising a lipid (e.g., a phospholipid), cholesterol, and a PEG functionalized lipid (e.g., a mPEG phospholipid). In certain embodiments the mPEG phospholipids comprises a C14-C18 phospholipid carbon chain from, and a PEG molecular weight from 350-5000 (e.g., MPEG 5000, MPEG 3000, MPEG 2000, MPEG 1000, MPEG 750, MPEG 550, MPEG 350, and the like). In certain embodiments the mPEG phospholipid comprises DSPE-PEG5000, DSPE-PEG3000, DSPE-PEG2000, DSPE-PEG1000, DSPE-PEG750, DSPE-PEG550, or DSPE-PEG350, PE-PEG5000, PE-PEG3000, PE-PEG2000, PE-PEG1000, PE-PEG750, PE-PEG550, PE-PEG350, and the like. MPEGs are commercially available (see, e.g., //avantilipids.com/product-category/products/polymers-polymerizable-lipids/mpeg-phospholipids).

In certain embodiments lipid bilayer comprises an mPEG phospholipid with a phospholipid C14-C18 carbon chain, and a PEG molecular weight ranging from about 350 Da to 5000 Da. In certain embodiments the lipid bilayer comprises PE-PEG_{2K}.

In certain embodiments the lipid bilayer comprises DPSC, cholesterol, and PE-PEG. In certain embodiments the ratio of DPSC:cholesterol:PE-PEG ranges from 40-90% DSPC:10%-50% Chol:1%-10% PE-PEG (molar ratio). In certain embodiments the ratio of DSPC:Chol:PE-PEG is about 60:40:3 molar ratio.

### Remote Loading of Drug Delivery Vehicles

In certain embodiments, the encapsulation of the GSK3 inhibitor (and other agents when present) in the nanoparticle can accomplished by using a “remote loading” strategy in which the addition of the drug (e.g., GSK3 inhibitor such as AZD1080) to LB-coated nanoparticles which achieves high loading levels using a pH gradient or an ion gradient capable of generating a pH gradient (see, e.g., Ogawa et al. (2009) J. Control. Rel. 1(5): 4-10; Fritze et al. (2006) Biochimica et Biophys Acta. 1758: 1633-1640). In general, the remote loading method involves adding a cargo-trapping reagent (e.g., a protonating reagent such as ammonium sulfate, TEA₈SOS, etc.) which can be added to the lipid biofilm prior to the sonication in the formation of LB coated nanoparticles.

Thus for example, in one illustrative, but non-limiting embodiment, the nanoparticles (e.g., MSNPs) are suspended in ammonium sulfate (e.g., 123 mM) in water by bath sonication. In an illustrative embodiment, as described in example 1, 40 mg of MSNP are resuspended in 1 mL of 123 mM ammonium sulfate. Materials for the lipid bilayer are dissolved in ethanol to provide a molar ratio of 60:40:3 for DPSC, cholesterol, and PE-PEG₂₀₀₀. Altogether, this amounts to 120 mg lipid (3× the MSNP mass), which is dissolved in 240 μL, ethanol at 65° C. The aqueous MSNP suspension is rapidly added to the lipid solution, followed by dilution with 5 mL of 123 mM ammonium sulfate. The crude lipid/MSNP mixture is probe sonicated, e.g., at 40% intensity, using two rounds of pulsing (each for 10 seconds, 5 second pause, 5 minute pulsing). The silicasome/liposome mixture is centrifuged for 5 minutes at 5,000×g to pellet large aggregates. The supernatant is collected and centrifuged at 21,000×g for 15 minutes to pellet the silicasomes. After washing and repeat of the centrifugation step, silicasomes are resuspended in, e.g., 0.9% NaCl solution.

In one embodiment, 100 μL of the silicasome suspension at 10 mg/mL in 0.9% NaCl (1 mg silicasomes) is added to the GSK3 inhibitor (e.g., AZD1080, AZD2858, LY2090314, 1-azakenpaullone) solutions, which can be prepared by adding 50, 100 or 200 μg of each inhibitor to 900 μL water. These drug mass quantities provided feed weight percentages of 5, 10, and 20%, in comparison to the silicasome mass.

Of course, this protocol is illustrative and non-limiting. Using this teaching, numerous other nanoparticle drug carriers comprising a nanoparticle surrounded by a lipid bilayer can be produced by one of skill in the art.

As explained above, in certain embodiments a cargo-trapping reagent (e.g., protonating agent) can be utilized to facilitate incorporation of the GSK3 inhibitor and in certain embodiments an additional cargo (e.g., DOX, MTX, OX, irinotecan etc.) into the LB coated nanoparticle.

In certain embodiments the cargo-trapping reagent can be selected to interact with a desired cargo. In some embodiments, this interaction can be an ionic or protonation reaction, although other modes of interaction are contemplated. The cargo-trapping agent can have one or more ionic sites, i.e., can be mono-ionic or poly-ionic. The ionic moiety can be cationic, anionic, or in some cases, the cargo-trapping agent can include both cationic and anionic moieties. The ionic sites can be in equilibrium with corresponding uncharged forms; for example, an anionic carboxylate (—COO⁻) can be in equilibrium with its corresponding carboxylic acid (—COOH); or in another example, an amine (—NH₂) can be in equilibrium with its corresponding protonated ammonium form (—NH₃⁺). These equilibriums are influenced by the pH of the local environment.

Likewise, in certain embodiments, the cargo, e.g., GSK3 inhibitor, can include one or more ionic sites. The cargo-trapping agent and cargo can be selected to interact inside the LB coated nanoparticle. This interaction can help retain the cargo within the nanoparticle until release of the cargo is desired. In some embodiments, the cargo can exist in a pH-dependent equilibrium between non-ionic and ionic forms. The non-ionic form can diffuse across the lipid bilayer and enter the vesicle or the pores of the MSNP. There, the cargo-trapping agent (e.g., a polyionic cargo-trapping agent) can interact with the ionic form of the cargo and thereby retain the cargo within the nanoparticle comprising the drug delivery vehicle, e.g., within the vesicle or within the pores of the nanoparticle (provided the ionic forms of the cargo and cargo-trapping agent have opposite charges). The interaction can be an ionic interaction, and can include formation of a precipitate. Trapping of cargo within the nanoparticle can provide higher levels of cargo loading compared to similar systems, e.g., drug delivery vehicles that omit the cargo-trapping agent, or liposomes that do include a trapping agent. Release of the cargo can be achieved by an appropriate change in pH to disrupt the interaction between the cargo and cargo-trapping agent, for example, by returning the cargo to its non-ionic state which can more readily diffuse across the lipid bilayer. In one embodiment, the cargo is AZD1080 and the cargo-trapping agent is ammonium sulfate.

The cargo trapping agent need not be limited to ammonium sulfate. In certain embodiments the cargo trapping comprises molecules like TEA₈SOS, citric acid, (NH₄)₂SO₄, and the like. Other trapping agents include, but are not limited to, ammonium salts (e.g., ammonium sulfate, ammonium sucrose octasulfate, ammonium α-cyclodextrin sulfate, ammonium β-cyclodextrin sulfate, ammonium γ-cyclodextrin sulfate, ammonium phosphate, ammonium α-cyclodextrin phosphate, ammonium β-cyclodextrin phosphate, ammonium γ-cyclodextrin phosphate, ammonium citrate, ammonium acetate, and the like), trimethylammonium salts (e.g., trimethylammonium sulfate, trimethylammonium sucrose octasulfate, trimethylammonium α-cyclodextrin sulfate, trimethylammonium β-cyclodextrin sulfate, trimethylammonium γ-cyclodextrin sulfate, trimethylammonium phosphate, trimethylammonium α-cyclodextrin phosphate, trimethylammonium β-cyclodextrin phosphate, trimethylammonium γ-cyclodextrin phosphate, trimethylammonium citrate, trimethylammonium acetate, and the like), triethylammonium salts (e.g., triethylammonium sulfate, triethylammonium sucrose octasulfate, triethylammonium α-cyclodextrin sulfate, triethylammonium β-cyclodextrin sulfate, triethylammonium γ-cyclodextrin sulfate, triethylammonium phosphate, triethylammonium α-cyclodextrin phosphate, triethylammonium β-cyclodextrin phosphate, triethylammonium γ-cyclodextrin phosphate, triethylammonium citrate, triethylammonium acetate, and the like).

It is also worth pointing out that transmembrane pH gradients can also be generated by acidic buffers (e.g. citrate) (Chou et al. (2003) J. Biosci. Bioengineer., 95(4): 405-408; Nichols et al. (1976) Biochimica et Biophysica Acta (BBA)-Biomembranes, 455(1): 269-271), proton-generating dissociable salts (e.g. (NH₄)₂SO₄) (Haran et al. (1993) Biochimica et Biophysica Acta (BBA)-Biomembranes, 1151(2): 201-215; Maurer-Spurej et al. (1999) Biochimica et Biophysica Acta (BBA)-Biomembranes, 1416(1): 1-10; Fritze et al. (2006) Biochimica et Biophysica Acta (BBA)-Biomembranes, 1758(10): 1633-1640), or ionophore-mediated ion gradients from metal salts (e.g. A23187 and MnSO₄) (Messerer et al. (2004) Clinical Cancer Res. 10(19): 6638-6649; Ramsay et al. (2008) Eur. J. Pharmaceut. Biopharmaceut. 68(3): 607-617; Fenske et al. (1998) Biochimica et Biophysica Acta (BBA)-Biomembranes, 1414(1): 188-204). Moreover, it is possible to generate reverse pH gradients for drug loading, such as use a calcium acetate gradient to improve amphiphilic weak acid loading in LB-MSNP, a strategy that has been utilized in liposomes (Avnir et al. (2008) Arthritis & Rheumatism, 58(1): 119-129).

Using the teachings provided herein, numerous other agents (e.g., other GSK3 inhibitors and/or other anti-cancer compounds) can be remote loaded (e.g., loaded using a cargo trapping agent) into the nanoparticle drug delivery systems described herein.

### Targeting Ligands.

In certain embodiments the drug delivery vehicles described herein can be conjugated to one or more targeting ligands, e.g., to facilitate specific delivery in endothelial cells, to cancer cells, to fusogenic ligands, e.g., to facilitate endosomal escape, ligands to promote transport across the blood-brain barrier, and the like.

In one illustrative, but non-limiting embodiment, the delivery vehicles described herein is conjugated to a fusogenic peptide such as histidine-rich HSWYG (H₂N-GLFHAIAHFIHGGWHGLIHGWYG-CO OH, (SEQ ID NO:1)) (see, e.g., Midoux et al., (1998) Bioconjug. Chem. 9: 260-267).

In certain embodiments delivery vehicles described herein are conjugated to one or more targeting ligand(s) that can include antibodies as well as targeting peptides. Targeting antibodies include, but are not limited to intact immunoglobulins, immunoglobulin fragments (e.g., F(ab)′₂, Fab, etc.) single chain antibodies, diabodies, affibodies, unibodies, nanobodies, and the like. In certain embodiments antibodies will be used that specifically bind a cancer marker (e.g., a tumor associated antigen). A wide variety of cancer markers are known to those of skill in the art. The markers need not be unique to cancer cells, but can also be effective where the expression of the marker is elevated in a cancer cell (as compared to normal healthy cells) or where the marker is not present at comparable levels in surrounding tissues (especially where the chimeric moiety is delivered locally).

Illustrative cancer markers include, for example, the tumor marker recognized by the ND4 monoclonal antibody. This marker is found on poorly differentiated colorectal cancer, as well as gastrointestinal neuroendocrine tumors (see, e.g., Tobi et al. (1998) Cancer Detection and Prevention, 22(2): 147-152). Other important targets for cancer immunotherapy are membrane bound complement regulatory glycoproteins CD46, CD55 and CD59, which have been found to be expressed on most tumor cells in vivo and in vitro. Human mucins (e.g. MUC1) are known tumor markers as are gp100, tyrosinase, and MAGE, which are found in melanoma. Wild-type Wilms' tumor gene WT1 is expressed at high levels not only in most of acute myelocytic, acute lymphocytic, and chronic myelocytic leukemia, but also in various types of solid tumors including lung cancer.

Acute lymphocytic leukemia has been characterized by the TAAs HLA-Dr, CD1, CD2, CD5, CD7, CD19, and CD20. Acute myelogenous leukemia has been characterized by the TAAs HLA-Dr, CD7, CD13, CD14, CD15, CD33, and CD34. Breast cancer has been characterized by the markers EGFR, HER2, MUC1, Tag-72. Various carcinomas have been characterized by the markers MUC1, TAG-72, and CEA. Chronic lymphocytic leukemia has been characterized by the markers CD3, CD19, CD20, CD21, CD25, and HLA-DR. Hairy cell leukemia has been characterized by the markers CD19, CD20, CD21, CD25. Hodgkin's disease has been characterized by the Leu-M1 marker. Various melanomas have been characterized by the HMB 45 marker. Non-hodgkins lymphomas have been characterized by the CD20, CD19, and Ia marker. And various prostate cancers have been characterized by the PSMA and SE10 markers.

In addition, many kinds of tumor cells display unusual antigens that are either inappropriate for the cell type and/or its environment, or are only normally present during the organisms' development (e.g., fetal antigens). Examples of such antigens include the glycosphingolipid GD2, a disialoganglioside that is normally only expressed at a significant level on the outer surface membranes of neuronal cells, where its exposure to the immune system is limited by the blood-brain barrier. GD2 is expressed on the surfaces of a wide range of tumor cells including neuroblastoma, medulloblastomas, astrocytomas, melanomas, small-cell lung cancer, osteosarcomas and other soft tissue sarcomas. GD2 is thus a convenient tumor-specific target for immunotherapies.

Other kinds of tumor cells display cell surface receptors that are rare or absent on the surfaces of healthy cells, and which are responsible for activating cellular signaling pathways that cause the unregulated growth and division of the tumor cell. Examples include (ErbB2) HER2/neu, a constitutively active cell surface receptor that is produced at abnormally high levels on the surface of breast cancer tumor cells.

Other useful targets include, but are not limited to CD20, CD52, CD33, epidermal growth factor receptor and the like.

An illustrative, but not limiting list of suitable tumor markers is provided in Table 4. Antibodies to these and other cancer markers are known to those of skill in the art and can be obtained commercially or readily produced, e.g. using phage-display technology. Such antibodies can readily be conjugated to the drug delivery vehicles (e.g., LB-coated nanoparticle) described herein, e.g., in the same manner that iRGD peptide is conjugated in Example 3.

TABLE 4
Illustrative cancer markers and associated references, all of which are incorporated
herein by reference for the purpose of identifying the referenced tumor markers.

| Marker | Reference |
|---|---|
| 5 alpha reductase | Délos et al. (1998) Int J Cancer, 75: 6 840-846 |
| α-fetoprotein | Esteban et al. (1996) Tumour Biol., 17(5): 299-305 |
| AM-1 | Harada et al. (1996) Tohoku J Exp Med., 180(3): 273-288 |
| APC | Dihlmannet al. (1997) Oncol Res., 9(3) 119-127 |
| APRIL | Sordat et al. (′998) J Exp Med., 188(6): 1185-1190 |
| BAGE | Böel et al. (1995) Immunity, 2: 167-175. |
| β-catenin | Hugh et al. (1999) Int J Cancer, 82(4): 504-11 |
| Bc12 | Koty et al. (1999) Lung Cancer, 23(2): 115-127 |
| bcr-abl (b3a2) | Verfaillie et al.(′996) Blood, 87(11): 4770-4779 |
| CA-125 | Bast et al. (′998) Int J Biol Markers, 13(4): 179-187 |
| CASP-8/FLICE | Mandruzzato et al. (1997) J Exp Med., 186(5): 785-793. |
| Cathepsins | Thomssen et al.(1995) Clin Cancer Res., 1(7): 741-746 |
| CD19 | Scheuermann et al. (1995) Leuk Lymphoma, 18(5-6): 385-397 |
| CD20 | Knox et al. (1996) Clin Cancer Res., 2(3): 457-470 |
| CD21, CD23 | Shubinsky et al. (1997) Leuk Lymphoma, 25(5-6): 521-530 |
| CD22, CD38 | French et al. (1995) Br J Cancer, 71(5): 986-994 |
| CD33 | Nakase et al. (1996) Am J Clin Pathol., 105(6): 761-768 |
| CD35 | Yamakawa et al. Cancer, 73(11): 2808-2817 |
| CD44 | Naot et al. (1997) Adv Cancer Res., 71: 241-319 |
| CD45 | Buzzi et al. (1992) Cancer Res., 52(14): 4027-4035 |
| CD46 | Yamakawa et al. (1994) Cancer, 73(11): 2808-2817 |
| CD5 | Stein et al. (1991) Clin Exp Immunol., 85(3): 418-423 |
| CD52 | Ginaldi et al. (1998) Leuk Res., 22(2): 185-191 |
| CD55 | Spendlove et al. (1999) Cancer Res., 59: 2282-2286. |
| CD59 (791Tgp72) | Jarvis et al. (1997) Int J Cancer, 71(6): 1049-1055 |
| CDC27 | Wang et al. (1999) Science, 284(5418): 1351-1354 |
| CDK4 | Wölfel et al. (1995) Science, 269(5228): 1281-1284 |
| CEA | Kass et al. (1999) Cancer Res., 59(3): 676-683 |
| c-myc | Watson et al. (1991) Cancer Res., 51(15): 3996-4000 |
| Cox-2 | Tsujii et al. (1998) Cell, 93: 705-716 |
| DCC | Gotley et al. (1996) Oncogene, 13(4): 787-795 |
| DcR3 | Pitti et al. (1998) Nature, 396: 699-703 |
| E6/E7 | Steller et al. (1996) Cancer Res., 56(21): 5087-5091 |
| EGFR | Yang et al. (1999) Cancer Res., 59(6): 1236-1243. |
| EMBP | Shiina et al. (1996) Prostate, 29(3): 169-176. |
| Ena78 | Arenberg et al. (1998) J. Clin. Invest., 102: 465-472. |
| FGF8b and FGF8a | Dorkin et al. (1999) Oncogene, 18(17): 2755-2761 |
| FLK-1/KDR | Annie and Fong (1999) Cancer Res., 59: 99-106 |
| Folic Acid Receptor | Dixon et al. (1992) J Biol Chem., 267(33): 24140-72414 |
| G250 | Divgi et al. (1998) Clin Cancer Res., 4(11): 2729-2739 |
| GAGE-Family | De Backer et al. (1999) Cancer Res., 59(13): 3157-3165 |
| gastrin 17 | Watson et al. (1995) Int J Cancer, 61(2): 233-240 |
| Gastrin-releasing | Wang et al. (1996) Int J Cancer, 68(4): 528-534 |
| hormone (bombesin) | |
| GD2/GD3/GM2 | Wiesner and Sweeley (1995) Int J Cancer, 60(3): 294-299 |
| GnRH | Bahk et al.(1998) Urol Res., 26(4): 259-264 |
| GnTV | Hengstler et al. (1998) Recent Results Cancer Res., 154: 47-85 |
| gp100/Pmel17 | Wagner et al. (1997) Cancer Immunol Immunother., 44(4): 239- |
| | 247 |
| gp-100-in4 | Kirkin et al. (1998) APMIS, 106(7): 665-679 |
| gp15 | Maeurer et al.(1996) Melanoma Res., 6(1): 11-24 |
| gp75/TRP-1 | Lewis et al.(1995) Semin Cancer Biol., 6(6): 321-327 |
| hCG | Hoermann et al. (1992) Cancer Res., 52(6): 1520-1524 |
| Heparanase | Vlodavsky et al. (1999) Nat Med., 5(7): 793-802 |
| Her2/neu | Lewis et al. (1995) Semin Cancer Biol., 6(6): 321-327 |
| Her3 | |
| HMTV | Kahl et al.(1991) Br J Cancer, 63(4): 534-540 |
| Hsp70 | Jaattela et al. (1998) EMBO J., 17(21): 6124-6134 |
| hTERT | Vonderheide et al. (1999) Immunity, 10: 673-679. 1999. |
| (telomerase) | |
| IGFR1 | Ellis et al. (1998) Breast Cancer Res. Treat., 52: 175-184 |
| IL-13R | Murata et al. (1997) Biochem Biophys Res Commun., 238(1): 90-94 |
| iNOS | Klotz et al. (1998) Cancer, 82(10): 1897-1903 |
| Ki 67 | Gerdes et al. (1983) Int J Cancer, 31: 13-20 |
| KIAA0205 | Guéguen et al. (1998) J Immunol., 160(12): 6188-6194 |
| K-ras, H-ras, | Abrams et al. (1996) Semin Oncol., 23(1): 118-134 |
| N-ras | |
| KSA | Zhang et al. (1998) Clin Cancer Res., 4(2): 295-302 |
| (CO17-1A) | |
| LDLR-FUT | Caruso et al. (1998) Oncol Rep., 5(4): 927-930 |
| MAGE Family | Marchand et al. (1999) Int J Cancer, 80(2): 219-230 |
| (MAGE1, | |
| MAGE3, etc.) | |
| Mammaglobin | Watson et al. (1999) Cancer Res., 59: 13 3028-3031 |
| MAP17 | Kocher et al. (1996) Am J Pathol., 149(2): 493-500 |
| Melan-A/ | Lewis and Houghton (1995) Semin Cancer Biol., 6(6): 321-327 |
| MART-1 | |
| mesothelin | Chang et al. (1996) Proc. Natl. Acad. Sci., USA, 93(1): 136-140 |
| MIC A/B | Groh et al.(1998) Science, 279: 1737-1740 |
| MT-MMP's, such as | Sato and Seiki (1996) J Biochem (Tokyo), 119(2): 209-215 |
| MMP2, MMP3, | |
| MMP7, MMP9 | |
| Mox1 | Candia et al. (1992) Development, 116(4): 1123-1136 |
| Mucin, such as MUC- | Lewis and Houghton (1995) Semin Cancer Biol., 6(6): 321-327 |
| 1, MUC-2, MUC-3, | |
| and MUC-4 | |
| MUM-1 | Kirkin et al. (1998) APMIS, 106(7): 665-679 |
| NY-ESO-1 | Jager et al. (1998) J. Exp. Med., 187: 265-270 |
| Osteonectin | Graham et al. (1997) Eur J Cancer, 33(10): 1654-1660 |
| p15 | Yoshida et al. (1995) Cancer Res., 55(13): 2756-2760 |
| P170/MDR1 | Trock et al. (1997) J Natl Cancer Inst., 89(13): 917-931 |
| p53 | Roth et al. (1996) Proc. Natl. Acad. Sci., USA, 93(10): 4781-4786. |
| p97/melanotransferrin | Furukawa et al. (1989) J Exp Med., 169(2): 585-590 |
| PAI-1 | Grøndahl-Hansen et al. (1993) Cancer Res., 53(11): 2513-2521 |
| PDGF | Vassbotn et al. (1993) Mol Cell Biol., 13(7): 4066-4076 |
| Plasminogen (uPA) | Naitoh et al. (1995) Jpn J Cancer Res., 86(1): 48-56 |
| PRAME | Kirkin et al. (1998) APMIS, 106(7): 665-679 |
| Probasin | Matuo et al. (1985) Biochem Biophys Res Commun., 130(1): 293- |
| | 300 |
| Progenipoietin | — |
| PSA | Sanda et al. (1999) Urology, 53(2): 260-266. |
| PSM | Kawakami et al.(1997) Cancer Res., 57(12): 2321-2324 |
| RAGE-1 | Gaugler et al.(1996) Immunogenetics, 44(5): 323-330 |
| Rb | Dosaka-Akita et al. (1997) Cancer, 79(7): 1329-1337 |
| RCAS1 | Sonoda et al.(1996) Cancer, 77(8): 1501-1509. |
| SART-1 | Kikuchi et al.(1999(Int J Cancer, 81(3): 459-466 |
| SSX gene | Gure et al. (1991) Int J Cancer, 72(6): 965-971 |
| Family | |
| STAT3 | Bromberg et al. (1999) Cell, 98(3): 295-303 |
| STn | Sandmaier et al. (1999) J Immunother., 22(1): 54-66 |
| (mucin assoc.) | |
| TAG-72 | Kuroki et al. (1990)Cancer Res., 50(16): 4872-4879 |
| TGF-α | Imanishi et al. (1989) Br J Cancer, 59(5): 761-765 |
| TGF-β | Picon et al. (1998) Cancer Epidemiol Biomarkers Prey, 7(6): 497- |
| | 504 |
| Thymosin β 15 | Bao et al. (1996) Nature Medicine. 2(12), 1322-1328 |
| IFN-α | Moradi et al. (1993) Cancer, 72(8): 2433-2440 |
| TPA | Maulard et al. (1994) Cancer, 73(2): 394-398 |
| TPI | Nishida et al.(1984) Cancer Res 44(8): 3324-9 |
| TRP-2 | Parkhurst et al. (1998) Cancer Res., 58(21) 4895-4901 |
| Tyrosinase | Kirkin et al. (1998) APMIS, 106(7): 665-679 |
| VEGF | Hyodo et al. (1998) Eur J Cancer, 34(13): 2041-2045 |
| ZAG | Sanchez et al. (1999) Science, 283(5409): 1914-1919 |
| p16INK4 | Quelle et al. (1995) Oncogene Aug. 17, 1995; 11(4): 635-645 |
| Glutathione | Hengstler (1998) et al. Recent Results Cancer Res., 154: 47-85 |
| S-transferase | |

Any of the foregoing markers can be used as targets for the targeting moieties comprising delivery vehicles described herein. In certain embodiments the target markers include, but are not limited to members of the epidermal growth factor family (e.g., HER2, HER3, EGF, HER4), CD1, CD2, CD3, CD5, CD7, CD13, CD14, CD15, CD19, CD20, CD21, CD23, CD25, CD33, CD34, CD38, 5E10, CEA, HLA-DR, HM 1.24, HMB 45, la, Leu-M1, MUC1, PMSA, TAG-72, phosphatidyl serine antigen, and the like.

The foregoing markers are intended to be illustrative and not limiting. Other tumor associated antigens will be known to those of skill in the art.

Where the tumor marker is a cell surface receptor, a ligand to that receptor can function as targeting moieties. Similarly, mimetics of such ligands can also be used as targeting moieties. Thus, in certain embodiments peptide ligands, and other ligands, can be used in addition to or in place of various antibodies. An illustrative, but non-limiting list of suitable targeting ligands is shown in Table 5. In certain embodiments any one or more of these peptides can be conjugated to a drug delivery vehicle described herein.

TABLE 5
Illustrative, but non-limiting ligands that target membrane
receptors expressed or overexpressed by various cancer cells.

| | | SEQ ID |
|---|---|---|
| Target Membrane Receptor | Targeting ligand | NO |
| Integrin receptor Aᵥβ₃ | c(RGDfK) | 2 |
| | c(RGDfC) | 3 |
| | c(RGDyC) | 4 |
| | RGD | |
| GFR | GE11 (YHWYGYTPQNVI) | 5 |
| GFR | GSG-KCCYSL | 6 |
| SSTR2 | Ostreotide | |
| GRP | QWAVGHML | 7 |
| CCK | DYMGWMDF | 8 |
| NT | RRPYIL | 9 |
| | RRPYILQLYENKPRRPYIL | 10 |
| LHRH | Gondaorelin | |
| GPRC family members | Antagonist G | |
| Tumor Cell Receptor | Targeting Ligand | |
| TfR | Tf | |
| EGFR | EGF | |
| FAR (FR-α) | FA | |
| FR-α | Methotrexate | |
| Sigma receptor | Anisamide | |
| Importing α and β receptors | TAT peptides | |
| IL-13Rα2 | IL-13 peptide | |
| HER2 | Anti-herceptin | |
| HER2/neu | Anti-HER2/neu | |
| ErbB2 | Anti-ErbB2 | |
| Mesothelin | Anti-ME1 | |
| CD105/endoglin | Anti-TRC105 | |
| NET | MABG | |
| NRP-1 | RGD-type peptide (RDGRC) | 11 |
| SA | ConA | |
| CD44 | HA | |
| αᵥβ₃ integrins | c(RGDyK) | |
| αᵥβ₃ integrins | cRGD | |
| αᵥβ₃ integrins | K7RGD; c-RGDFK | |
| | (SEQ ID NO: 12) | |
| αᵥβ₃ integrins | K₈(RGD)₂ | |
| | (SEQ ID NO: 13) | |
| αᵥβ₃ integrins | N₃GPLGRGRGDK-Ad | |
| | (SEQ ID NO: 14) | |
| αᵥβ₃ integrins | N₃RGDFFFFC | |
| | (SEQ ID NO: 15) | |
| αᵥβ₃ integrins | Thiolated-RGD | |
| (VCAM-1)R | Anti-(VCAM-1) | |
| VEGFR | VEGF | |

Tf: Transferrin; FA: Folic acid; EGFR: Epidermal growth factor; TAT: Transactivator of transcription; IL-13: Interleukin-13; MABG: metaaminobenzyl guanidine (meta-iodobenzylguanidine analogue); ConA: concanavalin A; c(RGD): Cyclic RGD (Arg-Gly-Asp); c(RGDyK): Cyclo(Arg-Gly-Asp-D-Phe-Lys); K₇RGD: linear RGD peptide sequence with 7 consecutive lysine residues; K₈(RGD)₂ cationic peptide containing 2 RGD sequences; VCAM-1: vascular cell adhesion molecule 1; VEGFR: Vascular endothelial growth factor; TfR: transferrin receptor; EGFR: epidermal growth factor receptor; FAR (FR-α): Folic acid receptor; IL-13Rα2: interleukin-13 receptor subunit alpha-2; HER2: epidermal growth factor receptor; ErbB2: Receptor tyrosine-protein kinase 2; NET: norepinephrine transporter; NRP-1: neuropilin receptors; SA: sialic acid; (VCAM-1)R: vascular cell adhesion molecule 1 receptor; VEGFR: Vascular endothelial growth factor receptor. c( ) indicates cyclopeptide. Lower case indicates “D” amino acid.

In certain embodiments the nanoparticle drug delivery vehicles described herein can be conjugated to moieties that facilitate stability in circulation and/or that hide the drug delivery vehicle from the reticuloendothelial system (RES) and/or that facilitate transport across a barrier (e.g., a stromal barrier, the blood brain barrier, etc.), and/or into a tissue. In certain embodiments the drug delivery vehicle are conjugated to transferrin or ApoE to facilitate transport across the blood brain barrier. In certain embodiments the drug delivery vehicle is conjugated to folate.

Methods of coupling the nanoparticle drug delivery vehicle to targeting (or other) agents are well known to those of skill in the art. Examples include, but are not limited to the use of biotin and avidin or streptavidin (see, e.g., U.S. Pat. No. 4,885,172 A), by traditional chemical reactions using, for example, bifunctional coupling agents such as glutaraldehyde, diimide esters, aromatic and aliphatic diisocyanates, bis-p-nitrophenyl esters of dicarboxylic acids, aromatic disulfonyl chlorides and bifunctional arylhalides such as 1,5-difluoro-2,4-dinitrobenzene; p,p′-difluoro m,m′-dinitrodiphenyl sulfone, sulfhydryl-reactive maleimides, and the like. Appropriate reactions which may be applied to such couplings are described in Williams et al. Methods in Immunology and Immunochemistry Vol. 1, Academic Press, New York 1967.

In one illustrative but non-limiting approach a peptide (e.g., iRGD) is coupled to the nanoparticle drug delivery vehicle by a lipid coupled to a linker (e.g., DSPE-PEG₂₀₀₀-maleimide), allowing thiol-maleimide coupling to the cysteine-modified peptide. It will also be recognized that in certain embodiments the targeting (and other) moieties can be conjugated to other moieties comprising the lipid bilayer. In certain embodiments possible to improve tumor delivery of the GSK3 inhibitor nanoparticle through co-administration (not conjugated) of the iRGD peptide to enhance particle transcytosis.

The former conjugates and coupling methods are illustrative and non-limiting. Using the teachings provided herein, numerous other moieties can be conjugated to the nanoparticle drug delivery vehicles described herein by any of a variety of methods.

### Combined Drug Delivery

In certain embodiments the drug delivery vehicles described herein can contain an additional cargo (in addition to a GSK3 inhibitor) in the cavities of the nanoparticle. In certain embodiments such additional cargoes comprise one or more cancer therapeutic agents. In certain embodiments the additional agents are cancer therapeutic agents capable of being remote loaded.

In certain embodiments the general characteristics of these cargo molecules include the following chemical properties:
(i) organic molecular compounds that include primary, secondary, or tertiary amine(s);
(ii) a pKa<11 to allow protonation and entrapment behind the LB (Zucker et al. (2009) J. Control. Release, 139(1): 73-80; Cern et al. (2012) J. Control. Release, 160(2): 147-157; Xu et al. (2014) Pharmaceut. Res. 31(10): 2583-2592);
(iii) a water solubility index of 5-25 mg/mL and amphipathic characteristics that allow diffusion across the LB;
(iv) an octanol/water partition coefficient or log P value of −3.0 to 3.0 (Zucker et al. (2009) J. Control. Release, 139(1): 73-80; Cern et al. (2012) J. Control. Release, 160(2): 147-157);
(v) suitable molecular weight with a geometric size less than MSNP pore size (2-8 nm), to allow entry into the MSNP pores (Li et al. (2012) Chem. Soc. Rev. 41(7): 2590-2605; Tang et al. (2012) Adv. Mat. 24(12): 1504-1534; Tarn et al. (2013) Acc. Chem. Res. 46(3): 792-801).

Remote loading doxorubicin with ammonium sulfate as a cargo trapping agent protonating agent) is described for example, in international patent publication No: WO 2018/.213631 (PCT/US2018/033265). This is illustrative, but non-limiting. In addition to DOX, there are other possible drugs that can be imported across the lipid bilayer of the drug delivery vehicles described herein. These include, but are not limited to, weak basic compounds, with medicinal chemical features. Such compounds include, but are not limited to alkaloids (e.g. irinotecan, topotecan, 10-hydroxycamptothecin, belotecan, rubitecan, vinorelbine, LAQ824, vinblastine, vincristine, homoharringtonine, trabectedin), anthracyclines (e.g. doxorubicin, epirubicin, pirarubicin, daunorubicin, rubidomycin, valrubicin, amrubicin), alkaline anthracenediones (e.g. mitoxantrone), alkaline alkylating agents (e.g. cyclophosphamide, mechlorethamine, temozolomide), purine or pyrimidine derivatives (e.g. 5-fluorouracil, 5′-deoxy-5-fluorouridine, gemcitabine, capecitabine) and protein kinase inhibitors (e.g., pazopanib, enzastaurin, vandetanib erlotinib, dasatinib, nilotinib, sunitinib, osimertinib, palbociclib, ribociclib), and the like.

Alternatively, or additionally, in certain embodiments, hydrophobic compounds can be incorporated into the lipid bilayer surrounding the nanoparticle. Thus, for example, paclitaxel can be incorporated in the lipid bilayer.

In certain embodiments the drug delivery vehicles described herein can contain an additional cargo (in addition to a GSK3 inhibitor) that is an inducer of immunogenic cell death (ICD inducer). Illustrative ICD inducers include, but are not limited to doxorubicin, oxaliplatin, anthracenedione, bleomycin, bortezomib, cisplatin, daunorubicin, docetaxel, epirubicin, idarubicin, mitoxanthrone, paclitaxel, R2016, and cyclophosphamide.

The foregoing compounds are illustrative and non-limiting. Using the teachings provided herein, numerous other additional cargoes can be incorporated in the drug delivery vehicles described herein.

### Pharmaceutical Formulations, Administration and Therapy

In some embodiments, the nanoparticle drug delivery vehicles described herein are administered alone or in a mixture with a physiologically-acceptable carrier (such as physiological saline or phosphate buffer) selected in accordance with the route of administration and standard pharmaceutical practice. For example, when used as an injectable, the nanoparticle drug delivery vehicles can be formulated as a sterile suspension, dispersion, or emulsion with a pharmaceutically acceptable carrier. In certain embodiments normal saline can be employed as the pharmaceutically acceptable carrier. Other suitable carriers include, e.g., water, buffered water, 0.4% saline, 0.3% glycine, 5% glucose and the like, including glycoproteins for enhanced stability, such as albumin, lipoprotein, globulin, etc. In compositions comprising saline or other salt-containing carriers, the carrier is preferably added following nanoparticle drug delivery vehicle formation. Thus, after the nanoparticle drug delivery vehicle is formed and loaded with suitable drug(s), the vehicles can be diluted into pharmaceutically acceptable carriers such as normal saline.

The pharmaceutical compositions may be sterilized by conventional, well-known sterilization techniques. The resulting aqueous solutions, suspensions, dispersions, emulsions, etc., may be packaged for use or filtered under aseptic conditions. In certain embodiments the nanoparticle drug delivery vehicles described herein are lyophilized, the lyophilized preparation being combined with a sterile aqueous solution prior to administration. The compositions may also contain pharmaceutically acceptable auxiliary substances as required to approximate physiological conditions, such as pH-adjusting and buffering agents, tonicity adjusting agents and the like, for example, sodium acetate, sodium lactate, sodium chloride, potassium chloride, calcium chloride, etc.

Additionally, in certain embodiments, the pharmaceutical formulation may include lipid-protective agents that protect lipids against free-radical and lipid-peroxidative damage on storage. Lipophilic free-radical quenchers, such as alpha-tocopherol and water-soluble iron-specific chelators, such as ferrioxamine, are suitable.

The concentration of the nanoparticle drug delivery vehicles in the pharmaceutical formulations can vary widely, e.g., from less than approximately 0.05%, usually at least approximately 2 to 5% to as much as 10 to 50%, or to 40%, or to 30% by weight and are selected primarily by fluid volumes, viscosities, etc., in accordance with the particular mode of administration selected. For example, the concentration may be increased to lower the fluid load associated with treatment. This may be particularly desirable in patients having atherosclerosis-associated congestive heart failure or severe hypertension. Alternatively, nanoparticle drug delivery vehicles composed of irritating lipids may be diluted to low concentrations to lessen inflammation at the site of administration. The amount of nanoparticle drug delivery vehicles administered will depend upon the particular drug used, the disease state being treated and the judgment of the clinician but will generally be between approximately 0.01 and approximately 50 mg per kilogram of body weight, preferably between approximately 0.1 and approximately 5 mg per kg of body weight.

In some embodiments, e.g., it is desirable to include polyethylene glycol (PEG)-modified phospholipids in the LB-coated nanoparticles or vesicles. Alternatively, or additionally, in certain embodiments, PEG-ceramide, or ganglioside Gm-modified lipids can be incorporated in the nanoparticle drug delivery vehicles described herein. Addition of such components helps prevent delivery vehicle aggregation and provides for increasing circulation lifetime and increasing the delivery of the loaded delivery vehicles to the target tissues. In certain embodiments the concentration of the PEG-modified phospholipids, PEG-ceramide, or Gm-modified lipids in the nanoparticle drug delivery vehicles will be approximately 1 to 15%.

In some embodiments, overall nanoparticle drug delivery vehicle charge is an important determinant in clearance of the vehicle from the blood. It is believed that highly charged delivery vehicles (e.g., zeta potential >+35 mV) will be typically taken up more rapidly by the reticuloendothelial system (see, e.g., Juliano (1975), Biochem. Biophys. Res. Commun. 63: 651-658 discussing liposome clearance by the RES). Drug delivery vehicles with prolonged circulation half-lives are typically desirable for therapeutic uses. For instance, in certain embodiments, drug delivery nanoparticle drug delivery vehicles that are maintained from 8 hrs, or 12 hrs, or 24 hrs, or greater are desirable.

In another example of their use, the nanoparticle drug delivery vehicles can be incorporated into a broad range of topical dosage forms including but not limited to gels, oils, emulsions, and the like, e.g., for the treatment of a topical cancer. For instance, in some embodiments the suspension containing the drug delivery vehicles is formulated and administered as a topical cream, paste, ointment, gel, lotion, and the like.

In some embodiments, pharmaceutical formulations comprising the nanoparticle drug delivery vehicles described herein additionally incorporate a buffering agent. The buffering agent may be any pharmaceutically acceptable buffering agent. Buffer systems include, but are not limited to citrate buffers, acetate buffers, borate buffers, and phosphate buffers. Examples of buffers include, but are not limited to citric acid, sodium citrate, sodium acetate, acetic acid, sodium phosphate and phosphoric acid, sodium ascorbate, tartaric acid, maleic acid, glycine, sodium lactate, lactic acid, ascorbic acid, imidazole, sodium bicarbonate and carbonic acid, sodium succinate and succinic acid, histidine, and sodium benzoate, benzoic acid, and the like.

In some embodiments, pharmaceutical formulations comprising the nanoparticle drug delivery vehicles described herein additionally incorporate a chelating agent. The chelating agent may be any pharmaceutically acceptable chelating agent. Chelating agents include but are not limited to ethylene diaminetetraacetic acid (also synonymous with EDTA, edetic acid, versene acid, and sequestrene), and EDTA derivatives, such as dipotassium edetate, disodium edetate, edetate calcium disodium, sodium edetate, trisodium edetate, and potassium edetate. Other chelating agents include citric acid (e.g., citric acid monohydrate) and derivatives thereof. Derivatives of citric acid include anhydrous citric acid, trisodiumcitrate-dihydrate, and the like. Still other chelating agents include, but are not limited to, niacinamide and derivatives thereof and sodium deoxycholate and derivatives thereof.

In some embodiments, pharmaceutical formulations comprising the nanoparticle drug delivery vehicles described herein additionally incorporate an antioxidant. The antioxidant may be any pharmaceutically acceptable antioxidant. Antioxidants are well known to those of ordinary skill in the art and include, but are not limited to, materials such as ascorbic acid, ascorbic acid derivatives (e.g., ascorbylpalmitate, ascorbylstearate, sodium ascorbate, calcium ascorbate, etc.), butylated hydroxy anisole, buylated hydroxy toluene, alkylgallate, sodium meta-bisulfate, sodium bisulfate, sodium dithionite, sodium thioglycollic acid, sodium formaldehyde sulfoxylate, tocopherol and derivatives thereof, (d-alpha tocopherol, d-alpha tocopherol acetate, dl-alpha tocopherol acetate, d-alpha tocopherol succinate, beta tocopherol, delta tocopherol, gamma tocopherol, and d-alpha tocopherol polyoxyethylene glycol 1000 succinate) monothioglycerol, sodium sulfite and N-acetyl cysteine. In certain embodiments such materials, when present, are typically added in ranges from 0.01 to 2.0%.

In some embodiments, pharmaceutical formulations comprising the nanoparticle drug delivery vehicles described herein are formulated with a cryoprotectant. The cryoprotecting agent may be any pharmaceutically acceptable cryoprotecting agent. Common cryoprotecting agents include, but are not limited to, histidine, polyethylene glycol, polyvinyl pyrrolidine, lactose, sucrose, mannitol, polyols, and the like.

In some embodiments, pharmaceutical formulations comprising the nanoparticle drug delivery vehicles described herein are formulated with an isotonic agent. The isotonic agent can be any pharmaceutically acceptable isotonic agent. This term is used in the art interchangeably with iso-osmotic agent and is known as a compound that is added to the pharmaceutical preparation to increase the osmotic pressure, e.g., in some embodiments to that of 0.9% sodium chloride solution, which is iso-osmotic with human extracellular fluids, such as plasma. Illustrative isotonicity agents include, but are not limited to, sodium chloride, mannitol, sorbitol, lactose, dextrose and glycerol.

In certain embodiments pharmaceutical formulations of the nanoparticle drug delivery vehicles described herein may optionally comprise a preservative. Common preservatives include, but are not limited to, those selected from the group consisting of chlorobutanol, parabens, thimerosol, benzyl alcohol, and phenol. Suitable preservatives include but are not limited to: chlorobutanol (e.g., 0.3-0.9% w/v), parabens (e.g., 0.01-5.0%), thimerosal (e.g., 0.004-0.2%), benzyl alcohol (e.g., 0.5-5%), phenol (e.g., 0.1-1.0%), and the like.

In some embodiments, pharmaceutical formulations comprising the nanoparticle drug delivery vehicles described herein are formulated with a humectant, e.g., to provide a pleasant mouth feel in oral applications. Humectants known in the art include, but are not limited to, cholesterol, fatty acids, glycerin, lauric acid, magnesium stearate, pentaerythritol, and propylene glycol.

In some embodiments, an emulsifying agent is included in the formulations, for example, to ensure complete dissolution of all excipients, especially hydrophobic components such as benzyl alcohol. Many emulsifiers are known in the art, e.g., polysorbate 60.

For some embodiments related to oral administration, it may be desirable to add a pharmaceutically acceptable flavoring agent and/or sweetener. Compounds such as saccharin, glycerin, simple syrup, and sorbitol are useful as sweeteners.

Administration

The nanoparticle drug delivery vehicles described herein can be administered to a subject (e.g., patient) by any of a variety of techniques.

In certain embodiments the nanoparticle drug delivery vehicles and/or pharmaceutical formulations thereof are administered parenterally, e.g., intraarticularly, intravenously, intraperitoneally, subcutaneously, or intramuscularly. In some embodiments, the pharmaceutical compositions are administered intravenously, intraarterially, or intraperitoneally by a bolus injection (see, e.g., U.S. Pat. Nos. 3,993,754; 4,145,410; 4,235,871; 4,224,179; 4,522,803; and 4,588,578 describing administration of liposomes). Particular pharmaceutical formulations suitable for this administration are found in Remington's Pharmaceutical Sciences, Mack Publishing Company, Philadelphia, Pa., 17th ed. (1985). Typically, the formulations comprise a solution of the nanoparticle drug delivery vehicles suspended in an acceptable carrier, preferably an aqueous carrier. As noted above, suitable aqueous solutions include, but are not limited to physiologically compatible buffers such as Hanks solution, Ringer's solution, or physiological (e.g., 0.9% isotonic) saline buffer and/or in certain emulsion formulations. The solution(s) can contain formulatory agents such as suspending, stabilizing and/or dispersing agents. In certain embodiments the active agent(s) can be provided in powder form for constitution with a suitable vehicle, e.g., sterile pyrogen-free water, before use. For transmucosal administration, and/or for blood/brain barrier passage, penetrants appropriate to the barrier to be permeated can be used in the formulation. These compositions may be sterilized by conventional, well-known sterilization techniques, or may be sterile filtered. The resulting aqueous solutions may be packaged for use as is, or lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc., e.g., as described above.

In other methods, the pharmaceutical formulations containing the nanoparticle drug delivery vehicles described herein may be contacted with the target tissue by direct application of the preparation to the tissue. The application may be made by topical, “open” or “closed” procedures. By “topical” it is meant the direct application of the pharmaceutical preparation to a tissue exposed to the environment, such as the skin, oropharynx, external auditory canal, and the like. Open procedures are those procedures that include incising the skin of a patient and directly visualizing the underlying tissue to which the pharmaceutical formulations are applied. This is generally accomplished by a surgical procedure, such as a thoracotomy to access the lungs, abdominal laparotomy to access abdominal viscera, or other direct surgical approaches to the target tissue. Closed procedures are invasive procedures in which the internal target tissues are not directly visualized but accessed via inserting instruments through small wounds in the skin. For example, the preparations may be administered to the peritoneum by needle lavage. Likewise, the pharmaceutical preparations may be administered to the meninges or spinal cord by infusion during a lumbar puncture followed by appropriate positioning of the patient as commonly practiced for spinal anesthesia or metrizamide imaging of the spinal cord. Alternatively, the preparations may be administered through endoscopic devices. In certain embodiments the pharmaceutical formulations are introduced via a cannula.

In certain embodiments the pharmaceutical formulations comprising the nanoparticle drug delivery vehicles described herein are administered via inhalation (e.g., as an aerosol). Inhalation can be a particularly effective delivery route for administration to the lungs and/or to the brain. For administration by inhalation, the nanoparticle drug delivery vehicles are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator may be formulated containing a powder mix of the compound and a suitable powder base such as lactose or starch.

In certain embodiments, the nanoparticle drug delivery vehicles described herein are formulated for oral administration. For oral administration, suitable formulations can be readily formulated by combining the drug delivery vehicles with pharmaceutically acceptable carriers suitable for oral delivery well known in the art. Such carriers enable the active agent(s) described herein to be formulated as tablets, pills, dragees, caplets, lozenges, gelcaps, capsules, liquids, gels, syrups, slurries, suspensions and the like, for oral ingestion by a patient to be treated. For oral solid formulations such as, for example, powders, capsules and tablets, suitable excipients can include fillers such as sugars (e.g., lactose, sucrose, mannitol and sorbitol), cellulose preparations (e.g., maize starch, wheat starch, rice starch, potato starch, gelatin, gum tragacanth, methyl cellulose, hydroxypropylmethyl-cellulose, sodium carboxymethylcellulose), synthetic polymers (e.g., polyvinylpyrrolidone (PVP)), granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric coated using standard techniques. The preparation of enteric-coated particles is disclosed for example in U.S. Pat. Nos. 4,786,505 and 4,853,230.

In various embodiments the nanoparticle drug delivery vehicles described herein can be formulated in rectal or vaginal compositions such as suppositories or retention enemas, e.g., containing conventional suppository bases such as cocoa butter or other glycerides. Methods of formulating active agents for rectal or vaginal delivery are well known to those of skill in the art (see, e.g., Allen (2007) Suppositories, Pharmaceutical Press) and typically involve combining the active agents with a suitable base (e.g., hydrophilic (PEG), lipophilic materials such as cocoa butter or Witepsol W45), amphiphilic materials such as Suppocire AP and polyglycolized glyceride, and the like). The base is selected and compounded for a desired melting/delivery profile.

The route of delivery of the nanoparticle drug delivery vehicles described herein can also affect their distribution in the body. Passive delivery of the drug delivery vehicles involves the use of various routes of administration e.g., parenterally, although other effective administration forms, such as intraarticular injection, inhalant mists, orally active formulations, transdermal iontophoresis, or suppositories are also envisioned. Each route produces differences in localization of the drug delivery vehicle.

Because dosage regimens for pharmaceutical agents are well known to medical practitioners, the amount of the liposomal pharmaceutical agent formulations that is effective or therapeutic for the treatment of a disease or condition in mammals and particularly in humans will be apparent to those skilled in the art. The optimal quantity and spacing of individual dosages of the formulations herein will be determined by the nature and extent of the condition being treated, the form, route and site of administration, and the particular patient being treated, and such optima can be determined by conventional techniques. It will also be appreciated by one of skill in the art that the optimal course of treatment, e.g., the number of doses given per day for a defined number of days, can be ascertained by those skilled in the art using conventional course of treatment determination tests.

Typically, the nanoparticle drug delivery vehicles described herein and/or pharmaceutical formations thereof described herein are used therapeutically in animals (including man) in the treatment of various cancers. In certain embodiments the drug delivery vehicles and/or pharmaceutical formations thereof described herein are particularly well suited in conditions that require: (1) repeated administrations; and/or (2) the sustained delivery of the drug in its bioactive form; and/or (3) the decreased toxicity with suitable efficacy compared with the free drug(s) in question. In various embodiments the nanoparticle drug delivery vehicles and/or pharmaceutical formations thereof are administered in a therapeutically effective dose. The term “therapeutically effective” as it pertains to the nanoparticle drug delivery vehicles described herein and formulations thereof means that GSK3 inhibitor contained therein, alone or in combination with other drugs, produces a desirable effect on the cancer. Such desirable effects include but are not limited to slowing and/or stopping tumor growth and/or proliferation and/or slowing and/or stopping proliferation of metastatic cells, reduction in size and/or number of tumors, and/or elimination of tumor cells and/or metastatic cells, and/or prevention of recurrence of the cancer following remission.

Exact dosages will vary depending upon such factors as the particular GSK3 inhibitor and the desirable medical effect, as well as patient factors such as age, sex, general condition, and the like. Those of skill in the art can readily take these factors into account and use them to establish effective therapeutic concentrations without resort to undue experimentation.

For administration to humans (or to non-human mammals) in the curative, remissive, retardive, or prophylactic treatment of diseases the prescribing physician will ultimately determine the appropriate dosage of the drug for a given human (or non-human) subject, and this can be expected to vary according to the age, weight, and response of the individual as well as the nature and severity of the patient's disease. In certain embodiments the dosage of the drug provided by the nanoparticle drug delivery vehicles can be approximately equal to that employed for the free drug. However as noted above, the nanoparticle drug delivery vehicles described herein can significantly reduce the toxicity of the drug(s) administered thereby and significantly increase a therapeutic window. Accordingly, in some cases dosages in excess of those prescribed for the free drug(s) will be utilized.

In certain embodiments, the dose of each of the drug(s) (e.g., GSK3 inhibitor(s)) administered at a particular time point will be in the range from about 1 to about 1,000 mg/m²/day, or to about 800 mg/m²/day, or to about 600 mg/m²/day, or to about 400 mg/m²/day. For example, in certain embodiments a dosage (dosage regiment) is utilized that provides a range from about 1 to about 350 mg/m²/day, 1 to about 300 mg/m²/day, 1 to about 250 mg/m²/day, 1 to about 200 mg/m²/day, 1 to about 150 mg/m²/day, 1 to about 100 mg/m²/day, from about 5 to about 80 mg/m²/day, from about 5 to about 70 mg/m²/day, from about 5 to about 60 mg/m²/day, from about 5 to about 50 mg/m²/day, from about 5 to about 40 mg/m²/day, from about 5 to about 20 mg/m²/day, from about 10 to about 80 mg/m²/day, from about 10 to about 70 mg/m²/day, from about 10 to about 60 mg/m²/day, from about 10 to about 50 mg/m²/day, from about 10 to about 40 mg/m²/day, from about 10 to about 20 mg/m²/day, from about 20 to about 40 mg/m²/day, from about 20 to about 50 mg/m²/day, from about 20 to about 90 mg/m²/day, from about 30 to about 80 mg/m²/day, from about 40 to about 90 mg/m²/day, from about 40 to about 100 mg/m²/day, from about 80 to about 150 mg/m²/day, from about 80 to about 140 mg/m²/day, from about 80 to about 135 mg/m²/day, from about 80 to about 130 mg/m²/day, from about 80 to about 120 mg/m²/day, from about 85 to about 140 mg/m²/day, from about 85 to about 135 mg/m²/day, from about 85 to about 135 mg/m²/day, from about 85 to about 130 mg/m²/day, or from about 85 to about 120 mg/m²/day. In certain embodiments the does administered at a particular time point may also be about 130 mg/m²/day, about 120 mg/m²/day, about 100 mg/m²/day, about 90 mg/m²/day, about 85 mg/m²/day, about 80 mg/m²/day, about 70 mg/m²/day, about 60 mg/m²/day, about 50 mg/m²/day, about 40 mg/m²/day, about 30 mg/m²/day, about 20 mg/m²/day, about 15 mg/m²/day, or about 10 mg/m²/day.

In certain embodiments, the dose administered may be higher or lower than the dose ranges described herein, depending upon, among other factors, the bioavailability of the composition, the tolerance of the individual to adverse side effects, the mode of administration and various factors discussed above. Dosage amount and interval may be adjusted individually to provide plasma levels of the composition that are sufficient to maintain therapeutic effect, according to the judgment of the prescribing physician. Skilled artisans will be able to optimize effective local dosages without undue experimentation in view of the teaching provided herein.

Multiple doses (e.g., continuous or bolus) of the compositions as described herein may also be administered to individuals in need thereof of the course of hours, days, weeks, or months. For example, but not limited to, 1, 2, 3, 4, 5, or 6 times daily, every other day, every 10 days, weekly, monthly, twice weekly, three times a week, twice monthly, three times a month, four times a month, five times a month, every other month, every third month, every fourth month, etc.

### Methods of Treatment.

In various embodiments methods of treatment using the nanoparticle drug delivery vehicles described herein and/or pharmaceutical formulation(s) comprising the nanoparticle drug delivery vehicles described herein are provided. In certain embodiments the method(s) comprise a method of treating a cancer. In certain embodiments the method can comprise administering to a subject in need thereof an effective amount of a nanoparticle drug delivery vehicle described herein, and/or a pharmaceutical formulation comprising the nanoparticle drug delivery vehicles.

In certain embodiments the nanoparticle drug delivery vehicles described herein (containing one or more GSK3 inhibitor(s)) and/or pharmaceutical formulation is a primary therapy in a chemotherapeutic regimen. In certain embodiments the nanoparticle drug delivery vehicle and/or pharmaceutical formulation is a component in an adjunct therapy in addition to chemotherapy using one or more other chemotherapeutic agents, and/or surgical resection of a tumor mass, and/or radiotherapy.

In certain embodiments the nanoparticle drug delivery vehicles and/or pharmaceutical formulation thereof is a component in a multi-drug chemotherapeutic regimen. In certain embodiments the multi-drug chemotherapeutic regimen comprises at least two drugs selected from the group consisting of irinotecan oxaliplatin (OX), 5-fluorouracil (5-FU), and leucovorin (LV). In certain embodiments the multi-drug chemotherapeutic regimen comprises at least three drugs selected from the group consisting of irinotecan oxaliplatin (OX), 5-fluorouracil (5-FU), and leucovorin (LV). In certain embodiments the multi-drug chemotherapeutic regimen comprises at least irinotecan oxaliplatin (OX), 5-fluorouracil (5-FU), and leucovorin (LV).

In various embodiments the nanoparticle drug delivery vehicles and/or pharmaceutical formulation(s) thereof described herein are effective for treating any of a variety of cancers. In certain embodiments the cancer is pancreatic ductal adenocarcinoma (PDAC). In certain embodiments the cancer is a cancer selected from the group consisting of acute lymphoblastic leukemia (ALL), acute myeloid leukemia (AML), adrenocortical carcinoma, AIDS-related cancers (e.g., Kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, glioblastoma, brain and spinal cord tumors, brain stem glioma, central nervous system atypical teratoid/rhabdoid tumor, central nervous system embryonal tumors, central nervous system germ cell tumors, craniopharyngioma, ependymoma, breast cancer, bronchial tumors, burkitt lymphoma, carcinoid tumors (e.g., childhood, gastrointestinal), cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm's tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sézary syndrome), Hodgkin, non-Hodgkin, primary central nervous system (CNS)), macroglobulinemia, Waldenström, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, chronic myeloid leukemia (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

In certain embodiments the nanoparticle drug delivery vehicles described herein are not conjugated to an iRGD peptide and the drug delivery vehicles are administered in conjunction with an iRGD peptide (e.g., the drug delivery vehicle and the iRGD peptide are co-administered as separate formulations).

In various embodiments of these treatment methods, the nanoparticle drug delivery vehicles described herein and/or pharmaceutical formulation is administered via a route selected from the group consisting of intravenous administration, intraarterial administration, intracerebral administration, intrathecal administration, oral administration, aerosol administration, administration via inhalation (including intranasal and intratracheal delivery, intracranial administration via a cannula, and subcutaneous or intramuscular depot deposition. In certain embodiments the drug delivery vehicles and/or pharmaceutical formulations thereof are administered as an injection, from an IV drip bag, or via a drug-delivery cannula. In various embodiments the subject is a human and in other embodiments the subject is a non-human mammal.

### Kits.

In certain embodiments, kits are provided containing reagents for the practice of any of the methods described herein. In certain embodiments the kit comprises a container containing a drug delivery vehicle described herein.

Additionally, in certain embodiments, the kits can include instructional materials disclosing the means of the use of the nanoparticle drug delivery vehicles described herein as a cancer therapeutic.

In addition, the kits optionally include labeling and/or instructional materials providing directions (e.g., protocols) for the use of the materials described herein, e.g., alone or in combination for the treatment of various cancers. Instructional materials can also include recommended dosages, description(s) of counterindications, and the like.

While the instructional materials in the various kits typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Nanodelivery of GSK3 Inhibitor for Immune Checkpoint Inhibition in Syngeneic Mouse Cancer Models

### Summary of Example 1

Antibody blockade of immune checkpoint receptors such as PD-1 is very effective for the treatment of several cancer types that are immune inflamed. However, it has also been shown that a small molecule inhibitor of GSK3, a signaling hub kinase, can interfere in the immune suppressive effect of the PD-1/PD-L1 axis in T-cells by inhibiting PD-1 expression. This provides an alternative approach to antibody use for interfering in this immune checkpoint pathway. Here, we demonstrate that the encapsulated delivery of a GSK3 inhibitor by a silicasome nanocarrier can be used for effective immunotherapy of a variety of cancers. Medicinal chemistry criteria were used to identify a weak basic compound, AZD1080, among a list of GSK3 inhibitors for remote loading into the porous interior of a lipid bilayer coated silicasome nanocarrier. Intravenous injection of encapsulated AZD1080 in mice resulted in significant tumor growth reduction in four syngeneic mouse tumor models, including two colorectal (MC38 and CT26) cancers, a pancreas (KPC), and a lung (LLC) cancer. Encapsulated AZD1080 also demonstrated robust anti-tumor immunity, as reflected by increased CD8⁺ density and perforin release, leading to enhanced tumor cell death. Not only was the therapeutic efficacy of encapsulated AZD1080 comparable to anti-PD-1 antibody, but the treatment was devoid of significant drug toxicity. These results provide proof-of-principal demonstration of the utility of encapsulated delivery of a GSK3 inhibitor for cancer immunotherapy, with the possibility to be used as a monotherapy or in combination with chemotherapy and other immunomodulatory agents.

As illustrated in this example, some of the therapeutic shortcomings of GSK3 inhibitors can be overcome by encapsulated drug delivery, which also provides a means of improving pharmacokinetics. In this regard, we have previously developed a lipid bilayer coated mesoporous silica nanoparticle (MSNP) platform that morphologically resembles a liposome, yet results in improved drug loading capacity, reduced leakage, and improved safety (19-22). This multifunctional carrier is also known as a “silicasome”, which is now a scalable technology that can be produced large quantities (e.g., −120 g batch sizes) (22) and can efficiently deliver up to 8% of the total injected drug dose to multiple tumor sites (19-21). Moreover, silicasomes are biodegradable and can be administered to mice as multiple doses of 100 mg/kg (22) or a single high dose of 1000 mg/kg (unpublished). In order to provide proof-of-principal testing of the utility of a silicasome carrier to interfere in PD-1 expression through GSK3i delivery, we developed a custom-designed carrier for encapsulation of AZD1080, a potent inhibitor that was identified by medicinal chemistry criteria for the potential to be loaded into the carrier at high doses, using the remote loading technique (23). We demonstrate that intravenous (IV) injection of the silicasome-AZD1080 carrier could significantly reduce tumor growth, with the same efficacy as anti-PD1 in two colorectal (MC38 and CT26) models, a pancreatic cancer (KPC), and a lung (LLC) cancer. The response was accompanied by enhanced tumor cell killing by cytotoxic T-cells showing decreased PD-1 expression. These findings provide proof-of principal demonstration of the utility of a nano-encapsulated GSK3 inhibitor for cancer immunotherapy, with the promise to be used as a monotherapy or in combination with other drug components.

### Results

Screening Selection of Commercially Available GSK3 Inhibitors to Produce Silicasome Carriers

We have established an effective drug loading approach for remote loading of weak-basic molecules into silicasomes through the inclusion of protonating agents (FIG. 2, panel A). Ammonium sulfate is an example FDA-approved protonating agent, which upon entrapment in the porous interior of MSNPs by a lipid bilayer, is capable of proton generation according to the following dissociation reaction: (NH₄)₂SO₄↔2NH₄⁺+SO₄²⁻↔2NH₃+2H⁺+SO₄²⁻(23). The inter-lysosomal release of H⁺ is capable of protonating weak-basic, amphipathic, chemical compounds that are capable of diffusing across the coated lipid bilayer. Protonation of science compounds renders them amphiphilic, preventing back diffusion across the lipid bilayer. Moreover, in its protonated state, the active pharmaceutical ingredients (API) bind to SO4²⁻ ions to form drug precipitates inside the carrier pores. We and others have used this entrapment procedure for the active import and encapsulation of numerous weak-basic chemotherapeutic agents (e.g., irinotecan) into liposomes and silicasomes (22, 24, 25). Not only is the remote loading technique highly efficient and simple to perform, but has received FDA approval for the development of the doxorubicin carrier, Doxil® (26).

Using medicinal chemistry criteria and quantitative structure-property relationship (QSPR) analysis, it is possible to screen drug libraries for chemical properties that allow remote loading against a proton gradient (23, 27). While classically these studies have been performed in liposomes, we hypothesized that the same principles would apply to lipid membrane-bound nanoparticles such as the silicasome (FIG. 2, panels A and B). Drugs that are expected to qualify for remote loading, are generally expected to exhibit the following chemical properties: (i) The compound needs to be relatively small to pass through the lipid bilayer (M.W.<1 kDa) and needs to have a geometric diameter smaller than the pore size (2-3 nm) to enter the MSNP; (ii) The molecule should have an isoelectric point between 7<pI<11 to allow lipid bilayer transit before entrapment; (iii) High water solubility coefficient (logS) in the range of 1-10 mg/mL at physiological pH (7.4); and (iv) The molecule must be amphipathic to pass through a lipid bilayer, a property that can be evaluated by a partition coefficient (clogP). Thus, while high clogP values (>5) characterize compounds that are difficult to solubilize with increased propensity to be independent in a lipid bilayer, compounds with low logP values (<0) are hydrophilic and less likely to pass through the lipid membrane. These selection criteria were applied to 17 commercially available GSK3 inhibitors (Table 6), the chemical structures of which are shown in FIG. 10.

TABLE 6
Relevant chemical properties of commercially available GSK3 inhibitors, arranged
according to IC₅₀ values, chemical abstracts service number (CAS No), and MW,
as provided by chemical vendors. The MarvinSketch ChemAxon software suite was
used, to calculate the in silico parameters, cLogP (at pH 7.4), the pI and LogS
(at pH 7.4). The compounds highlighted in bold fulfill the prediction criteria
for chemical characteristics amenable to remote loading. These compounds were
acquired to conduct remote loading studies, in addition to the GSK3 inhibitor,
SB415286, that was previously used to assess the impact on PD-1 expression (14).

| | IC50 | MW | | | LogS | |
|---|---|---|---|---|---|---|
| GSK3 Inhibitor | (nM) | (g/mol) | cLogP | pI | @pH 7.4 | CAS NO. |
| CHIR99021 | 6.7 | 465.3 | 3.99 | 9.69 | −6.86 | 252917-06-9 |
| SB-216753 | 34 | 371.2 | 4.23 | N/A | −5.39 | 280744-09-4 |
| CHIR98014 | 0.58 | 486.323 | 1.63 | 10.16 | −7.43 | 556813-39-9 |
| TWS119 | 40 | 318.336 | 3.05 | 6.71 | −6.03 | 601514-19-6 |
| Tideglusib | 60 | 334.39 | 4.72 | N/A | −6.16 | 865854-05-3 |
| SB-416286 | 78 | 359.72 | 2.19 | N/A | −4.91 | 264218-23-7 |
| BIO | 5 | 356.17 | 4.37 | 4.1 | −7.04 | 667463-62-9 |
| AZD2858 | 68 | 453.52 | 1.15 | 10.2 | −2.4 | 486424-20-8 |
| AZD1080 | 31 | 334.4 | 1.9 | 7.67 | −2.91 | 612487-72-6 |
| AR-A014418 | 104 | 308.31 | 2.2 | N/A | −3.93 | 487021-52-3 |
| TDZD-8 | 2000 | 222.3 | 1.19 | N/A | −2.39 | 327036-89-5 |
| LY2090314 | 0.9 | 512.545 | 1.18 | 7.54 | −5.22 | 603288-22-8 |
| 2-D08 | 11 | 270.24 | 2.06 | N/A | −3.22 | 144707-18-6 |
| BIO-acetoxime | 10 | 398.21 | 2.71 | 6.42 | −6.35 | 667463-85-6 |
| IM-12 | 53 | 377.41 | 2.99 | N/A | −5.39 | 1129669-05-1 |
| 1-Azakenpaullone | 18 | 328.2 | 2.77 | 7.75 | −4.09 | 676596-65-9 |
| Indirubin-3′- | 22 | 277.3 | 1.48 | 4.45 | −6.05 | 160807-49-8 |
| monoxime | | | | | | |

Among these, two compounds provided good matches based on all the listed criteria (AZD2858 and AZD1080), while two others were moderate matches (LY2090314 and 1-Azakenpaullone) (Table 6). All four were purchased for experimental validation of their remote loading efficiency.

Remote Loading of GSK3 Inhibitors and Silicasome Characterization

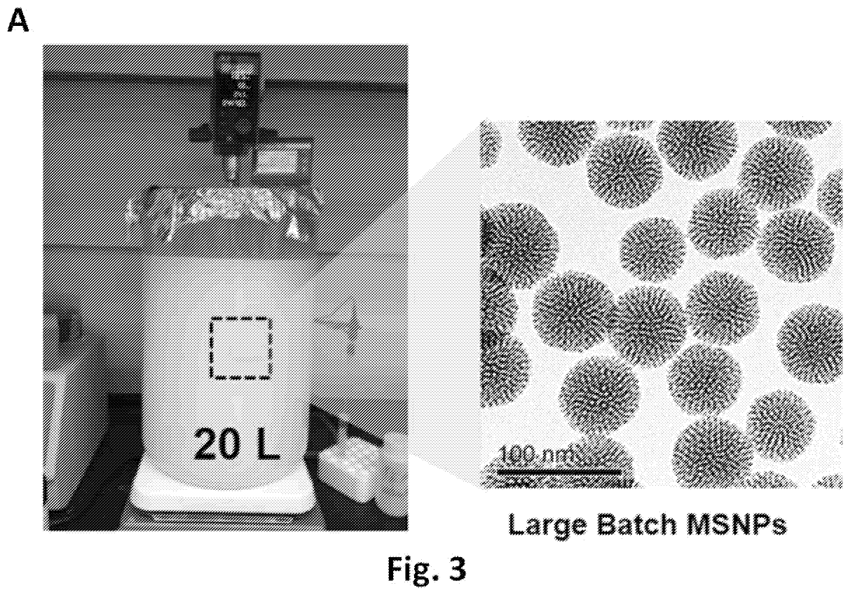

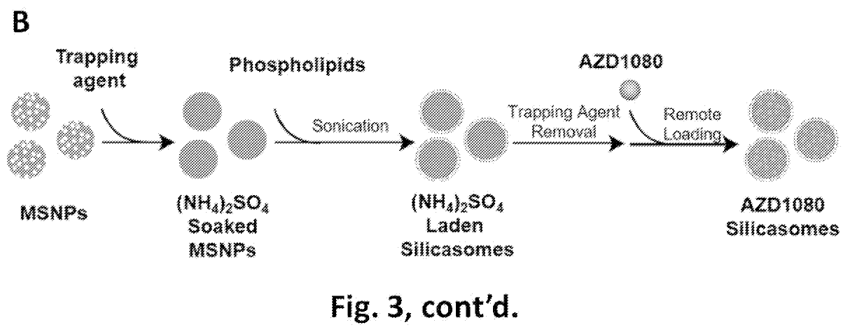

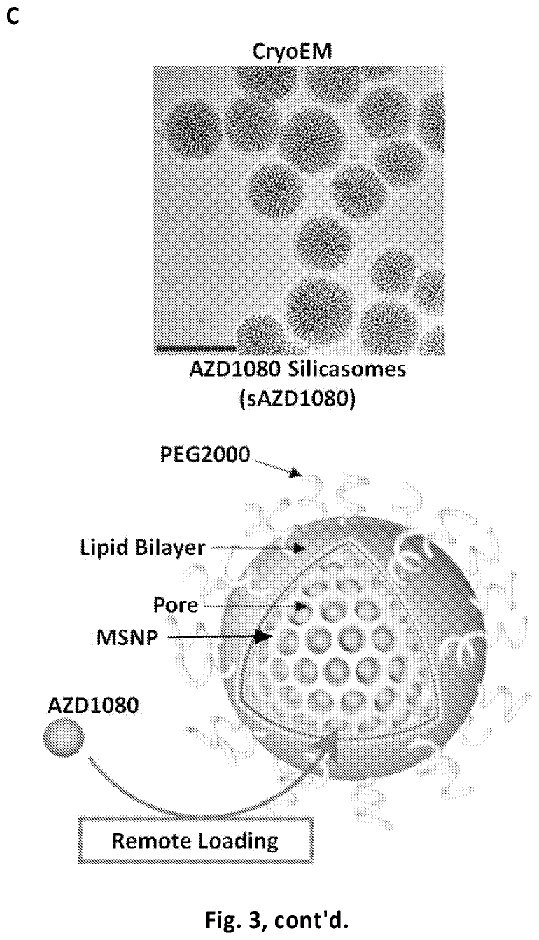

The overall scheme for the synthesis of silicasome carriers to deliver GSK3 inhibitors is outlined in FIG. 3. Briefly, a large batch (i.e. 120 g) synthesis of bare particles was carried out as previously, yielding ˜70 nm MSNPs (FIG. 3, panel A) (22). 20 mg/mL bare particles at were soaked in a 123 mM ammonium sulfate solution, which generates a pH of 5 inside the particles (FIG. 2, panel A) (28). 20 mg soaked-in MSNPs were then used together with an ethanol suspended lipid solution, comprised of a mixture of 60 mg DSPC:Cholesterol:PE-PEG2000 (molar ratio of 6:4:0.3), for coating with a lipid bilayer by a flow through sonication procedure (FIG. 3, panel B). Following the removal of the non-encapsulated trapping agent, AZD2858, AZD1080, LY2090314, and 1-Azakenpaullone were dissolved in 0.9% saline and incubated with the silicasome carrier to assess the loading capacity in response to different feed weight percentages (FIG. 11). This demonstrated that while the encapsulation efficiency of 1-Azakenpaullone was comparatively low, AZD1080 and AZD2858, LY2090314 could be loaded at moderate efficiency. However, AZD2858 showed poor solubility at feed weight percentages >20%. In contrast, AZD1080 showed good aqueous solubility and displayed good encapsulation efficiency of 62.5% at a feed weight of 5%; this allowed a total loading capacity of 8.1 w/w % (see, e.g., Table 7).

TABLE 7
Illustrative silicasome physical characteristics, encapsulation
efficiency, and loading capacity for AZD1080. DLS, zeta
potential, and encapsulation data are representative
of 3 samples (SD = standard deviation).

| Size | 160.9 ± 6.3 | NM |
|---|---|---|
| PDI | 0.092 ± 0.51 | |
| Zeta Potential | −14.28 ± 4.6 | mV |
| Encapsulation Efficiency | 62.5 ± 4.6% | |
| Loading Capacity | 8.1 ± 0.6 | (w/w %) |

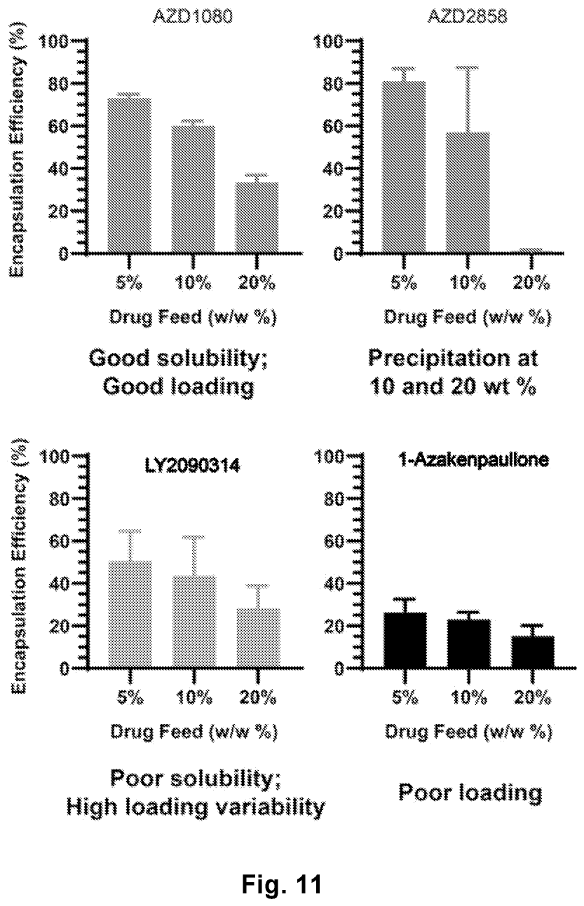

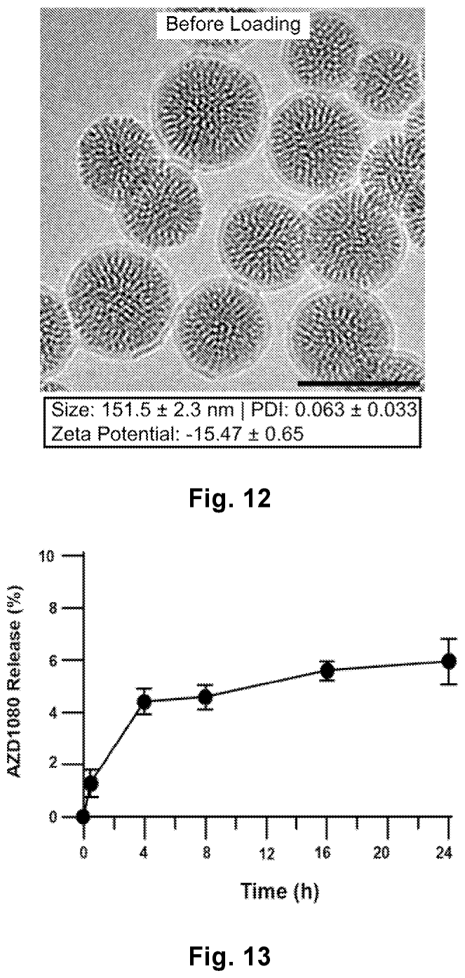

AZD1080 remote loading did not appreciably impact the silicasome size or morphology, as demonstrated by cryogenic transmission electron microscopy (cryoTEM) (FIG. 3, panel C, and FIG. 12). However, AZD1080 loading did result in a slight increase in the hydrodynamic diameter of silicasomes (151.5 nm vs 160.9 nm) by DLS. There was a slow release of AZD1080 from the particles, amounting to −5% cumulative release over 24 hours in 100% fetal bovine serum at 37° C. (Supplemental FIG. 13). Based on above loading and performance characteristics, we opted to use the fully characterized AZD1080-silicasomes for further in vitro and in vivo studies of its possible effect on PD-1 expression

T-Cell Screening to Assess PD-1 Inhibition by Free and Encapsulated GSK3 Inhibitors

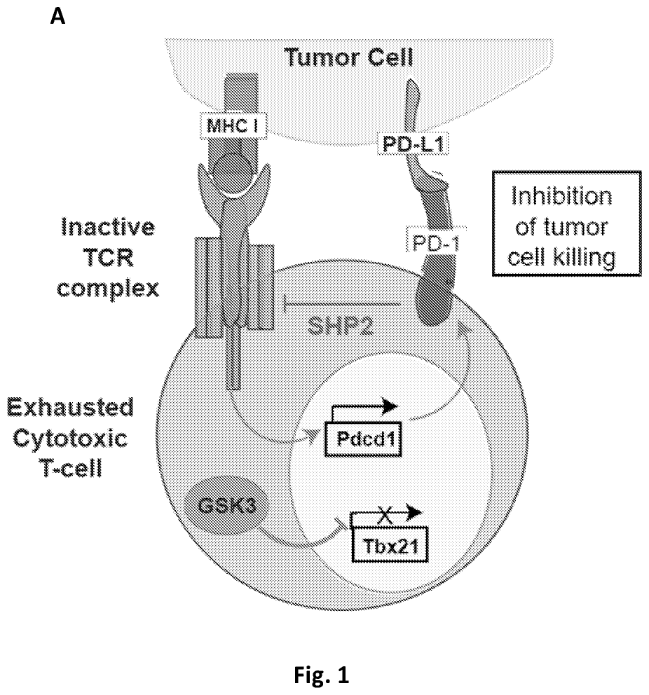

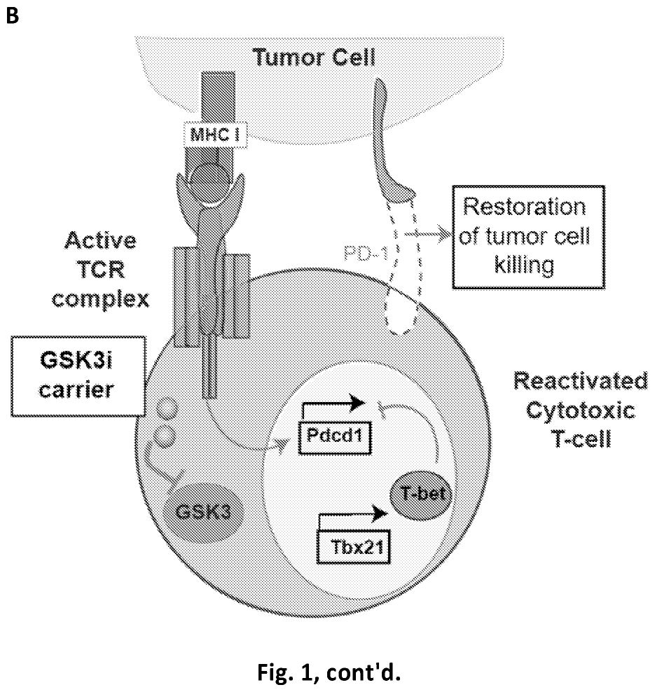

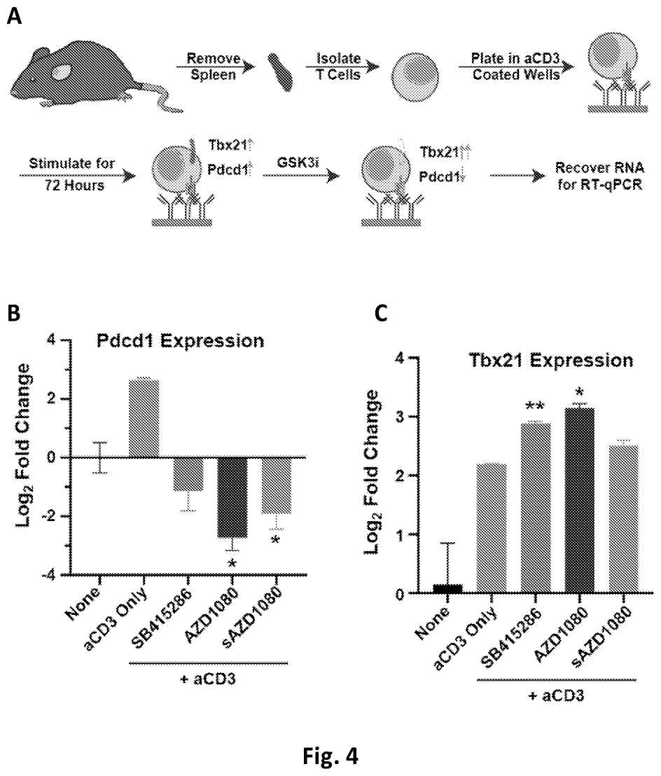

In order to confirm the ability of AZD1080 to inhibit PD-1 expression in vitro, we utilized an RT-qPCR assay, described by Taylor et al (14) for demonstrating how the GSK3 inhibitor effect of SB415286 interfered in Pdcd1 expression along with increased Tbx21 abundance in anti-CD3 activated T-cells (FIG. 4, panel A). Compared to a ˜2.2-fold reduction in Pdcd1 mRNA expression by SB415286, the comparable decreases by free AZD1080 and silicasome-encapsulated AZD1080 (sAZD1080) were ˜6- and ˜3.7-fold, respectively (FIG. 4, panel B). These results were the inverse of increased Tbx21 mRNA expression in response to SB415286, AZD1080 and sAZD1080 (FIG. 4, panel C). This is compatible with the translational expression of T-bet, a transcription factor that interferes in PD-1 expression (FIG. 1, panel B). All considered, these data met the previously published literature expectations and demonstrate that a GSK3 SMI, AZD1080, in either free or encapsulated form, is capable of inhibiting Pdcd1 mRNA expression in vitro.

sAZD1080 Inhibits Tumor Growth by Restoring Cytotoxic T-Cells Responses in a Syngeneic Colon Cancer Model (MC38)

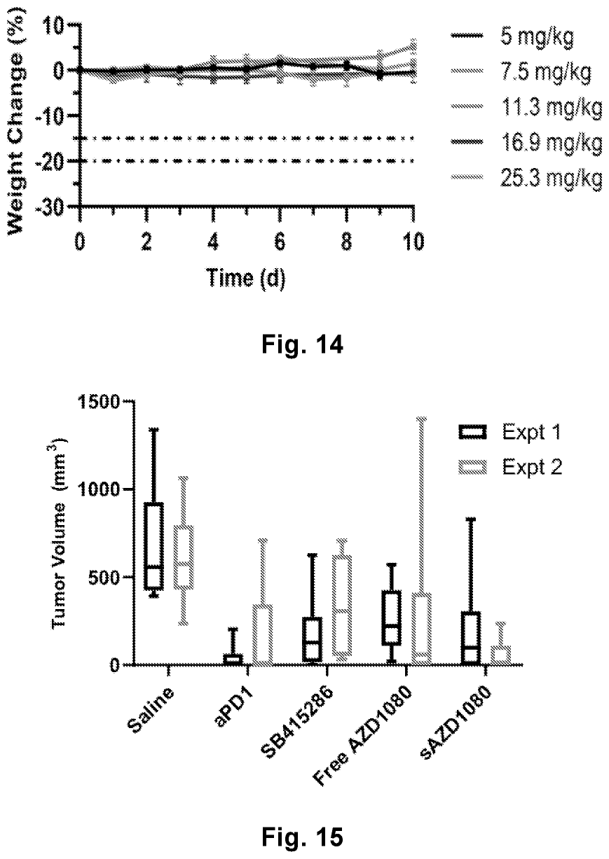

In order to establish a safe working concentration for murine experiments on AZD1080, we used a previously established dose of 5 mg/kg for the treatment of an Alzheimer's-like disease process in rats (29) for planning of a maximum tolerated dose assessment (FIG. 14). While it was not possible to obtain dose escalation beyond 25.3 mg/kg due to the lack of solubility of free AZD1080, we could ascertain that dose escalation from 5 to 25.3 mg/kg was devoid of significant weight loss, abnormal behavior or posture, mortality or other adverse events. This prompted us to continue the use of the established dose of 5 mg/kg, for a total of three injections in our animal studies.

The first assessment of sAZD1080 was carried out in a MC38 colon cancer model, which is regarded as a highly responsive to treatment by anti-PD1 monoclonal antibodies (Table 8).

TABLE 8
Syngeneic tumor models in this study.

| | | | | | aPD-1 | |
|---|---|---|---|---|---|---|
| | | Syngeneic | Method of | Mutational | monotherapy | Major |
| Name | Type | Mouse | Generation | Load* | responsiveness*** | Oncogenes*** |
| MC38 | Colon | C57BL/6 | Chemical | ~3,400 | Responsive | Alk, Braf, |
| | Carcinoma | | (DMH) | | | Erbb4, Jak2, |
| | | | | | | Jak3, Pten, |
| | | | | | | Ptpn11, Stat3, |
| | | | | | | Trp53 |
| KPC | Pancreatic | B6/129 | Genetic | 2** | Poor | Kras, Trp53** |
| | Ductal | | | | | |
| | Adeno- | | | | | |
| | carcinoma | | | | | |
| CT26 | Colon | BALB/C | Chemical | ~3,300 | Moderate | Brca2, Cdk4, |
| | Carcinoma | | (NMU) | | | Erbb3, Fgfr1, |
| | | | | | | Kras, Pdgfra |
| LLC | Lung | C57BL/6 | Spontaneous | ~2,300 | Poor | Alk, Apc, |
| (LL/2) | Carcinoma | | | | | Cdk4, Csf1r, |
| | | | | | | Erbb3, Flt1, |
| | | | | | | Flt3, Jak2, |
| | | | | | | Jak3, Met |

*Mosley et al. Cancer Immunol. Res. 2017
**Lee et al. Curr. Protoc. Pharmacol. 2016
***Charles River Syngeneic model Immunotherapyu Responsiveness Data

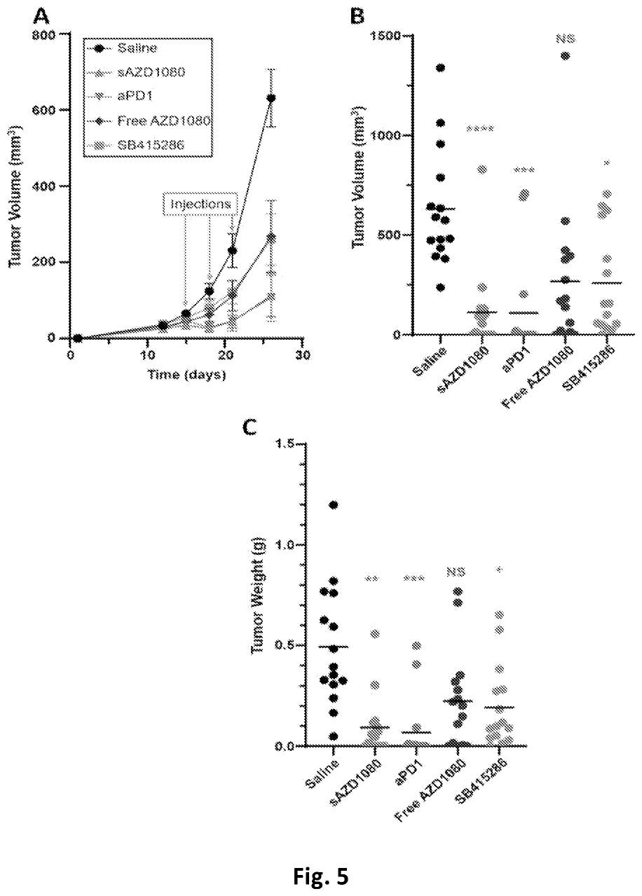

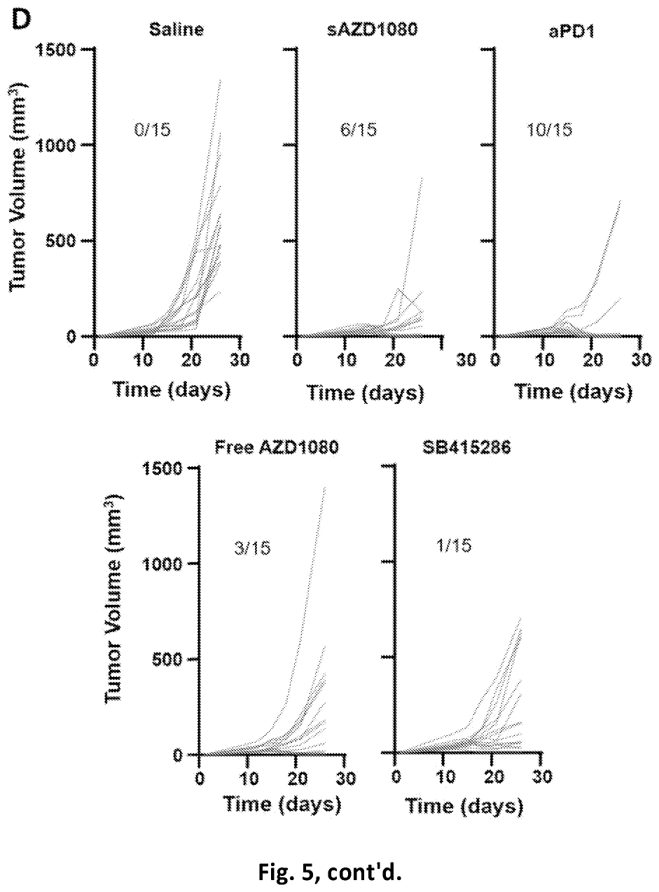

Following the establishment of subcutaneous tumor growth, treatment with free AZD1080, sAZD1080, anti-PD-1 antibody, SB415286, or saline commenced 10 days after implantation. Animals treated with sAZD1080 was IV injected with 5 mg/kg of the drug (corresponding to a particle dose of ˜62 mg/kg). Controls included animals receiving intraperitoneal (IP) doses of 5, 8 and 4 mg/kg, respectively, of free AZD1080, SB415286 and anti-PD-1. Treatment was repeated every three days for a total of three administrations. Mice were sacrificed four days after the final treatment. The study was executed as two separate experiments, using animal group sizes of n=6 and n=9, respectively. Although each experiment yielded significant data in its own right, there were no significant differences between the experiments in terms of tumor volumes, prompting us to combine the data for pooled analysis, which will be used in the subsequent discussion (FIG. 15). The first set of analyses was performed on the tumor volume growth curves (FIG. 5, panel A), impact on final tumor volumes (FIG. 5, panel B) and tumor weight (FIG. 5, panel C) at the time of sacrifice. These data demonstrate that all the treatment modalities yielded significant tumor growth inhibition, except for free AZD1080. The most robust tumor volume reduction was obtained with sAZD1080 (FIG. 5, panel B), while the biggest decrease in weight was obtained with anti-PD1 (FIG. 5, panel C). Inspection of the cumulative growth curves (spaghetti plots) for each animal demonstrate the intra-group variances, including tumor disappearance in some animals (FIG. 5, panel D). Thus, while all the tumors in the saline group expanded exponentially, the number of “tumor-free” animals were 1/15 during treatment with SB415286, 3/15 for free AZD1080, 6/15 for sAZD1080, and 10/15 for anti-PD-1 treatment.

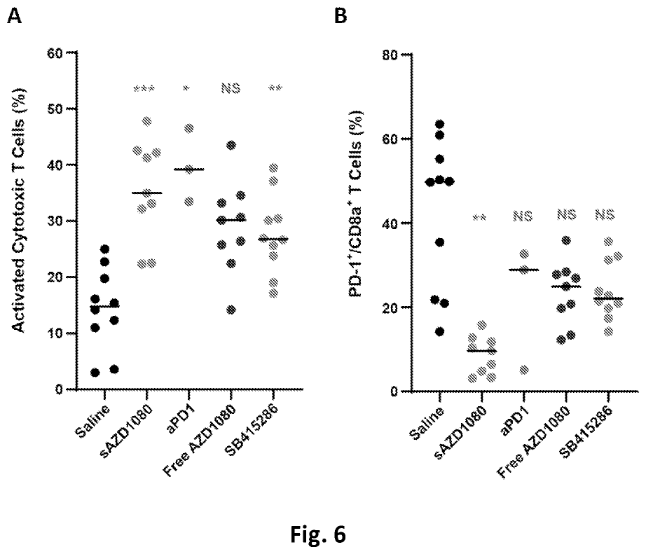

In order to address the change in the immunological landscape with treatment, we asked whether sAZD1080 could impact the immune parameters that underpin treatment efficacy for immune checkpoint blocking antibodies. These include the CD8⁺ cell number, the abundance of PD-1 expression on these cells, perforin release and the generation of cancer cell death, as reflected by activated caspase 3. For the first analysis, tumor cell digests were used to assess the number of CD8⁺/CD107a⁺/granzyme B⁺ lymphocytes by flow cytometry (14, 30). This demonstrated a significant increase in the number of activated CD8⁺ T-cells in all treatment groups compared to the saline control, except for free AZD1080 (FIG. 6, panel A). In order to assess the status of PD-1 expression on CD8+ cells, flow cytometry was used to assess the % of CD8⁻/PD-1⁺ cells in the tumor digests (14, 31). This demonstrated that while anti-PD-1, SB415286 or free AZD1080 did not impact the cell population, sAZD1080 administration could significantly suppress the % of CD8⁺/PD-1⁺ cells (FIG. 6, panel B). It is important to mention, however, that due to their disappearance, fewer anti-PD-1 treated tumors were available for analysis, which may explain why this data point did not reach significance.

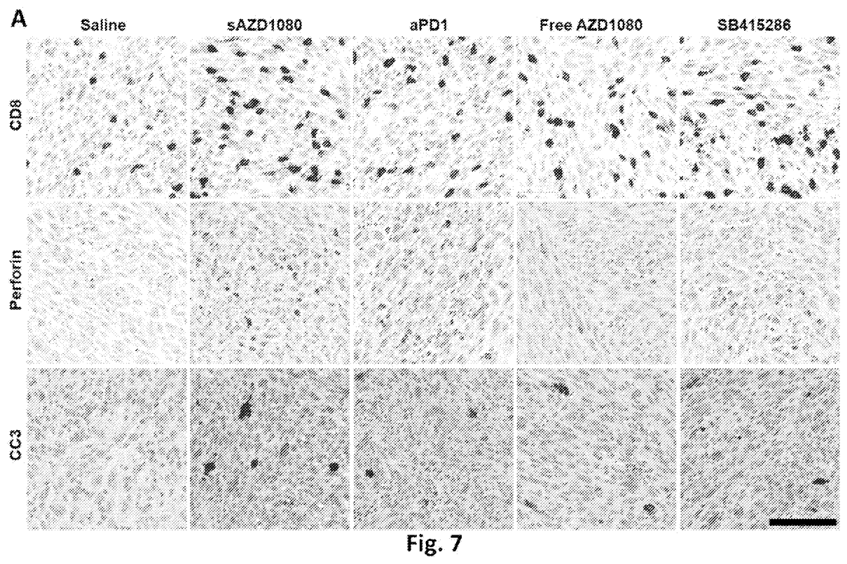

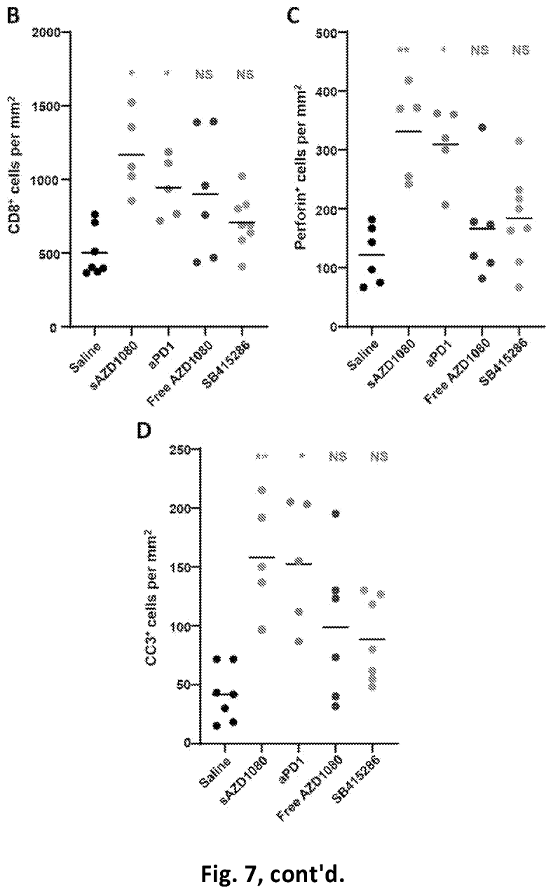

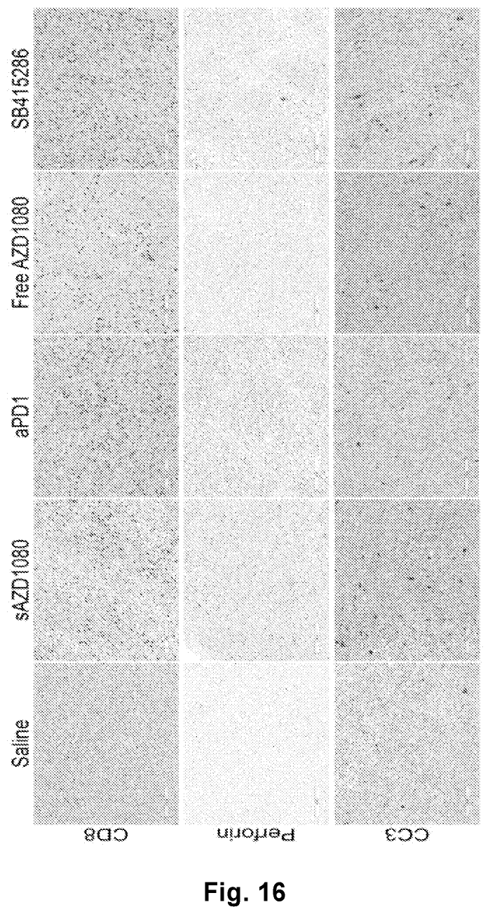

The flow cytometry data were supported through the use of immunohistochemistry (IHC) staining of embedded tumor sections to visualize CD8, perforin and cleaved caspase 3 expression (FIG. 7, panel A), further assisted by image software analysis to derive quantitative information (FIG. 7, panels B-D). Low magnification images appear in FIG. 16. Treatment with sAZD1080 and anti-PD-1 resulted in a doubling of the number of CD8⁺ cells per mm², which was statistically significant (FIG. 7, panel B). Perforin expression, an indicator of cytotoxic T-cell maturation and ability to induce tumor lysis (32, 33), was also significantly increased by the same treatment groups (FIG. 7, panel C). Moreover, these therapies increased the rate of tumor cell death as reflected by cleaved caspase 3 (CC3) staining (FIG. 7, panel D). No statistically significant change in CC3 expression was observed in mice receiving free GSK3 inhibitors. All considered, the flow and IHC data provide strong support for the involvement of cytotoxic T-cell activity in decreasing tumor volume (FIG. 6).

Safety of the sAZD1080 Treatment Platform

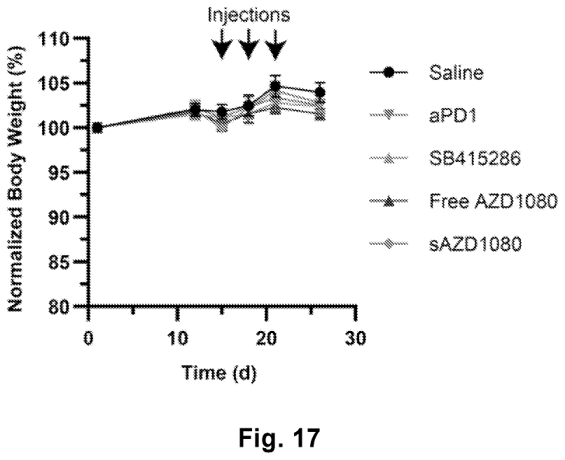

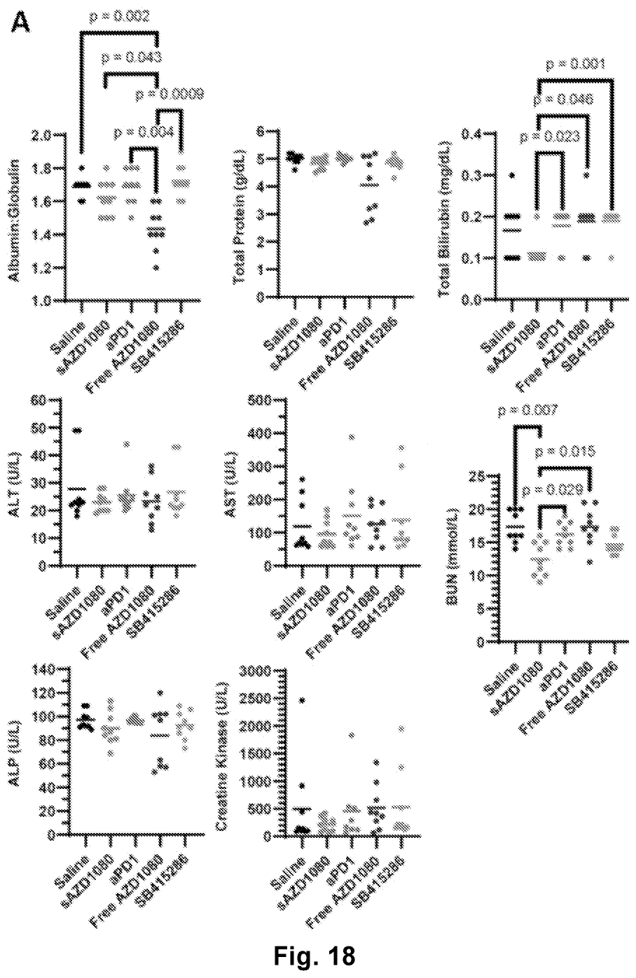

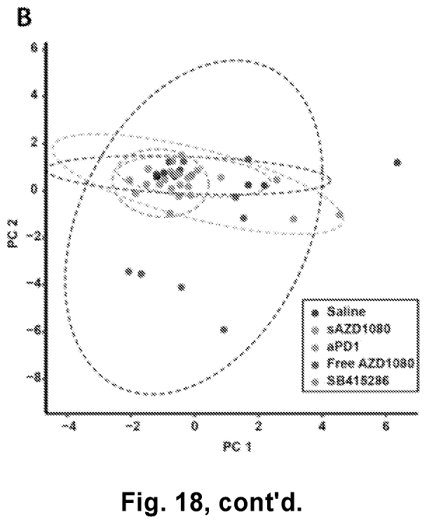

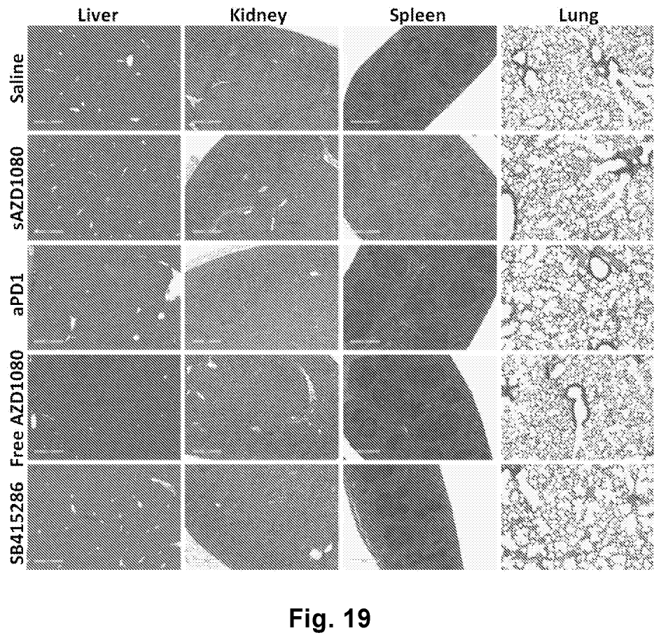

We have previously demonstrated the intrinsic safety of the silicasome platform in the treatment of pancreatic cancer, including the role of the supported lipid bilayer in preventing premature release and toxicity by irinotecan (20, 21, 25). While there is sparse literature on the use of AZD1080 in animal studies, this agent had to be discontinued in a phase I clinical trial in humans due to nephrotoxicity (15). However, our safety analysis in mice failed to show any changes in animal weight or evidence of toxicity during treatment with sAZD1080 (FIG. 16). This includes the absence of significant effects on blood chemistry, even though free AZD1080 could be seen to depress the albumin:globulin ratio of which is of unknown significance (FIG. 17, panel A). Interestingly, both encapsulated and free AZD1080 administration was also associated with reductions in total bilirubin and BUN levels, which do not reflect toxicity. Principal component analysis indicated that free AZD1080 was responsible for more variation in blood chemistry than sAZD1080 or other treatments (17, panel B). Finally, H&E staining of a number of organs collected at the time of sacrifice, failed to show any histological evidence of tissue damage, including for the kidney (FIG. 18). All considered, no evidence of toxicity was observed in sAZD1080 use.

The Favorable Immunotherapeutic Effects of sAZD1080 Also Applies to Syngeneic Models for Pancreatic, Lung and Colon Cancer

While the MC38 model is highly responsive to the administration of immune checkpoint blocking antibodies, most animal and human cancers are either unresponsive to treatment or only partially responsive (34-36). To obtain a more comprehensive picture of the efficacy of sAZD1080 versus anti-PD-1 treatment, three additional syngeneic cancer models were investigated, namely another colon cancer (CT26), Lewis lung cancer (LLC) and a Kras-derived pancreatic cancer (KPC). These animal tumor models differ with respect to the cancer cell type, tumor origin, mutational load, oncogene expression, and tumor escape mechanisms (Table 8). Moreover, in contrast to the characterization of MC38 as “highly responsive” to anti-PD-1, the responsiveness of CT26 to the same treatment was labeled as “moderate”, with KPC and LLC classified as “poorly responsive”.

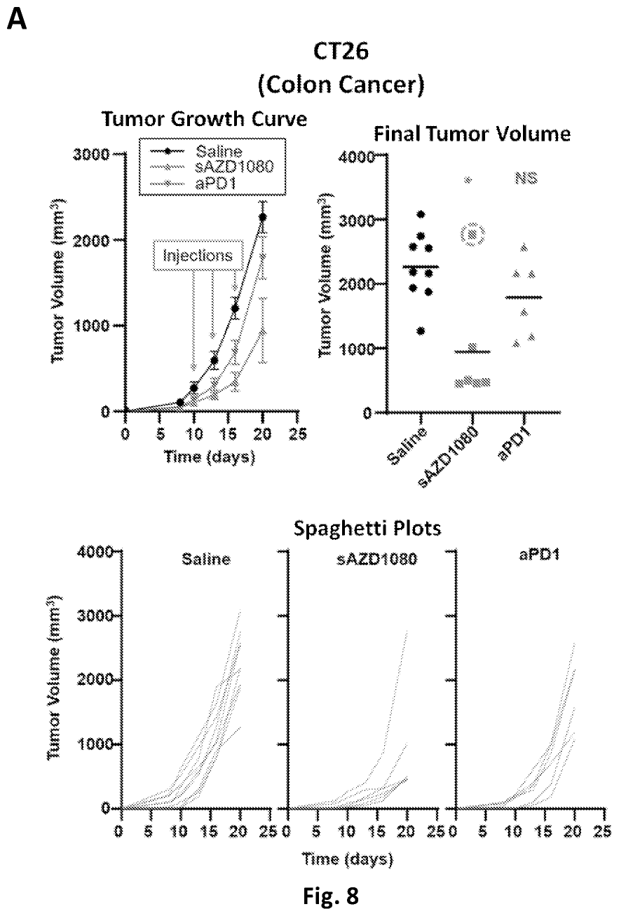

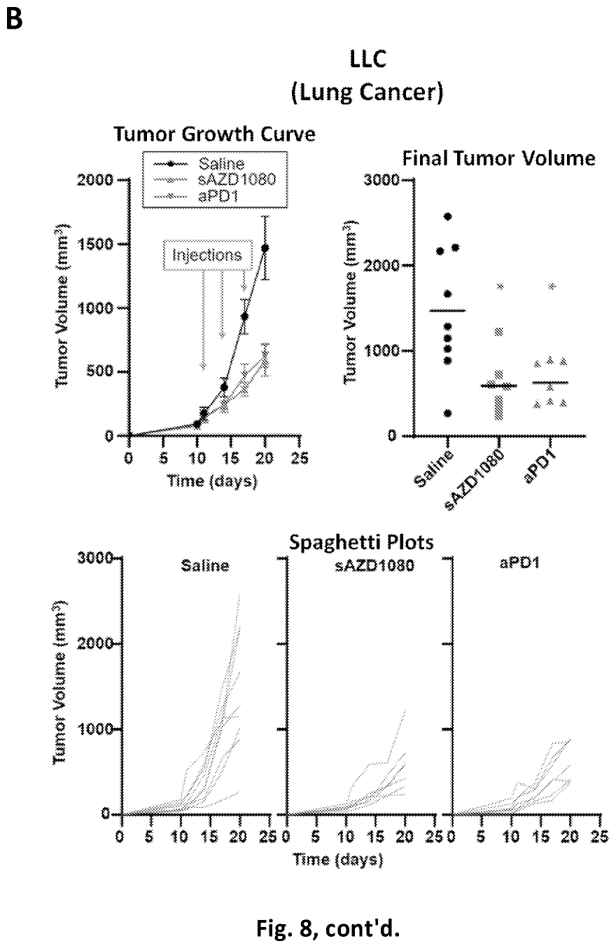

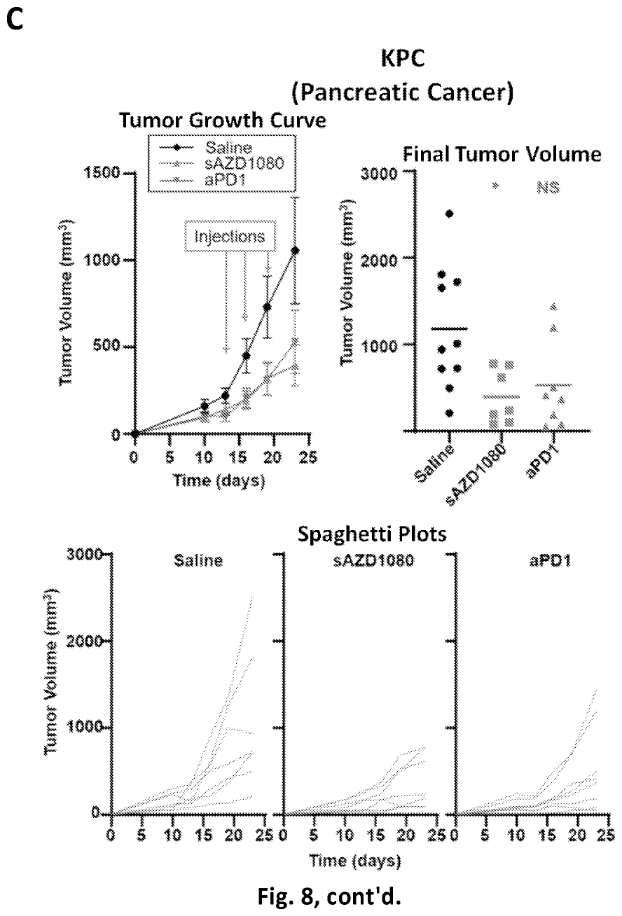

The first comparison of sAZD1080 to anti-PD-1 was carried out in CT26, another colon cancer model that was derived from a chemical-induced cancer with a high mutational load (3,300 neoantigens) (36). In contrast to MC38, CT26 is considered moderately responsive to anti-PD-1 therapy (34, 36). Systemic administration of above therapeutic agents, used at similar doses and treatment intervals as in the MC38 model, demonstrated response differences in tumor growth, interference (FIG. 8, panel A). Thus, while sAZD1080 reduced tumor growth by ˜58%, which was significantly different from saline (p=0.0425), the tumor growth reduction of ˜21% by anti-PD-1 treatment was not statistically significant (p=0.3656). Although there was no statistical significance in the final tumor volume size of sAZD1080 vs. anti-PD-1 (p=0.2349), the removal of one outlier in the former treatment group (green dotted circle) could be shown to result in a statistically significant difference (p=0.0079). In contrast to the robustness of the immunotherapy response in MC38, none of treatments resulted in tumor disappearance in CT26.

The next investigation was into the LLC model. These tumor cells are derived from a spontaneously developing lung cancer and also exhibit a high mutational load of ˜2,300 mutations (36). This model is considered poorly responsive to anti-PD-1 monotherapy (37). The experimentation in LLC demonstrated good responsiveness to both sAZD1080 and anti-PD-1, with statistically significant differences in tumor growth inhibition and final tumor volume (compared to saline) (FIG. 8, panel B). Final tumor volume was reduced by ˜60% by sAZD1080 treatment (p=0.0250) and ˜57% (p=0.0277) by anti-PD-1.

The last comparison of sAZD1080 with anti-PD-1 was carried out in a pancreatic ductal adenocarcinoma (PDAC) model, known for its complex TME and treatment resistance to multiple treatment modalities (38, 39). The KPC model was established using a cell line derived from in a transgenic Kras^{LSL-G12D/+}; Trp53^{LSL-R172H/+}; Pdx-1-Cre animal (40). Similar to human PDAC, KPC have a low mutational load and is poorly responsive to immunotherapy (39), including anti-PD-1 monotherapy (41, 42). Systemic administration of sAZD1080 led to significant tumor growth inhibition (p=0.0258), with a ˜66% reduction in tumor volume compared to saline (FIG. 8, panel C). However, while anti-PD-1 treatment reduced the tumor volume size by ˜55%, the growth inhibition in KPC did not achieve statistical significance (p=0.1126).

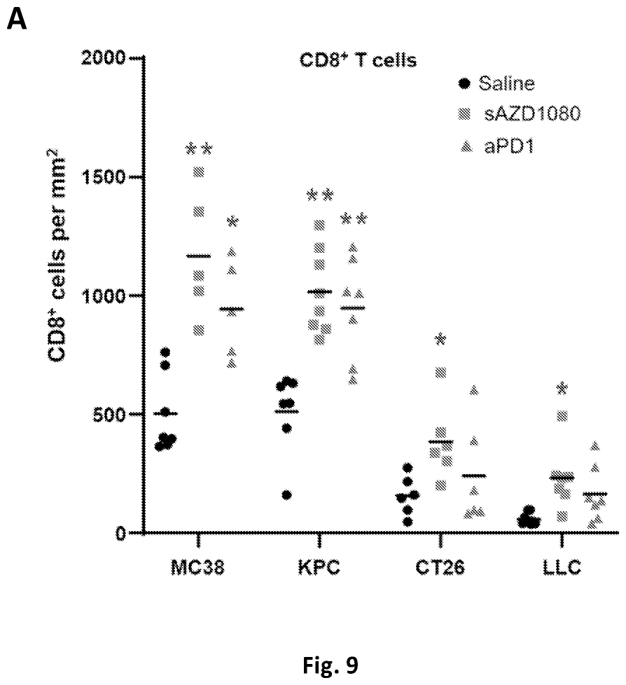

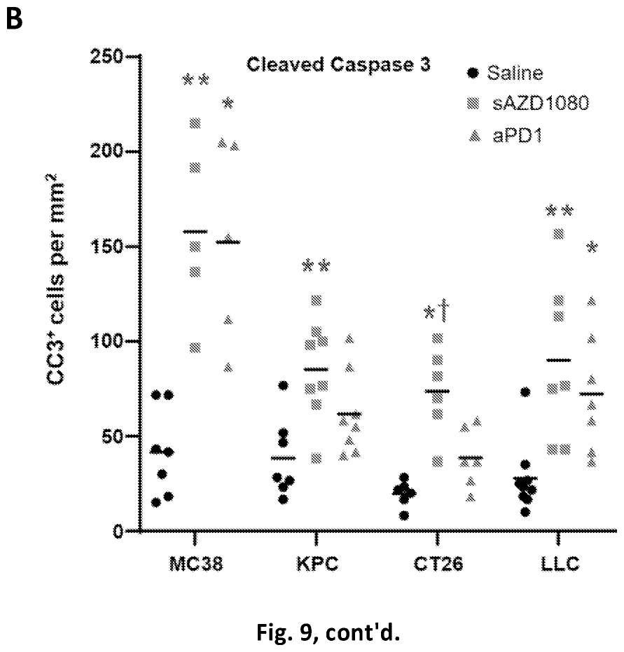

All considered, our data demonstrate that encapsulated sAZD1080 delivery can induce significant growth inhibition in four different cancers, including in animal models where anti-PD-1 treatment did not make a significant impact. In order to demonstrate whether, similar to MC38, the responses to sAZD1080 in other tumor types are mediated by cytotoxic T-cells, IHC analysis was performed to assess CD8 recruitment and tumor cell death (CC3 staining) (FIG. 9, panel A). This demonstrated that sAZD1080 treatment resulted in a significant increase in the number of CD8⁺ CTLs for all the cancers (FIG. 9, panel A). While similar responsiveness was seen for MC38 and KPC during anti-PD-1 treatment, this therapy did not result in significant CTL recruitment in CT26 and LLC (FIG. 9, panel A). These results were also compatible with the increase in CC3 staining. Thus, while sAZD1080 treatment resulted in a significant increase in tumor cell death for all tumors, anti-PD1 did not impact CCL3 staining in KPC and CT26 models (FIG. 9, panel B). Moreover, the response to sAZD1080 in CT26 was significantly higher than the anti-PD-1 treatment group (p<0.05). All considered, these data support the notion that encapsulated delivery of the GSK3 inhibitor, AZD1080, induce significant tumor growth inhibition by increasing the abundance and tumor cell killing effects of CD8⁺ CTLs in heterogeneous cancer models.

### Discussion

In this example, we demonstrate the utility of a silicasome carrier to deliver the GSK3 inhibitor, AZD1080, to the cancer site of multiple syngeneic animal tumor models. AZD1080 was selected from a panel of GSK3 inhibitors by medicinal chemistry criteria to predict remote loading into silicasomes by a proton gradient. AZD1080 was found to inhibit Pdcd1 expression in murine T-cells in free as well as encapsulated drug form. Following the establishment of an MC38 colon cancer model in immunocompetent C57 BL/6 mice, we could demonstrate that systemic administration of sAZD1080 was associated with a significant reduction of tumor growth, resulting 6 of 15 mice to become tumor-free. This response outcome was comparable to the tumor-inhibiting effects of anti-PD-1 antibody. In contrast, free AZD1080 had no significant effect on tumor growth inhibition compared to the negative control. Flow cytometric analysis further demonstrated that sAZD1080 administration could significantly increase the number of activated CD8+ T-cells. Moreover, IHC analysis revealed a significant increase in in perforin, granzyme B and CC3 staining at the tumor site, again of similar magnitude to anti-PD-1. In contrast, free GSK3 inhibitors did not significantly impact cytotoxic killing of tumor cells. Additional assessment of sAZD1080 in a second colon cancer (CT26), a lung cancer (LLC) and a pancreatic cancer (KPC) model confirmed the carrier's significant tumor growth inhibitory effects as a result of cytotoxic T-cell recruitment and induction of lytic tumor cell death. All considered, sAZD1080 was capable of generating at least comparable rates of tumor growth inhibition as anti-PD-1 antibody for all tumors tested, demonstrating the potential utility of an encapsulated SMI of GSK3 to supplement or compete with antibody mediated immune checkpoint therapy.

PD-1/PD-L1 receptor engagement serves as a potent immunosuppressive signaling pathway and provides an attractive target for cancer immunotherapy (43). Newly generated cytotoxic T-cells are capable of accomplishing tumor cell killing during engagement of the TCR signaling complex, which is responsible for the release of cytotoxic granules containing perforin and granzyme B (44). However, a variety of bystander mechanisms can contribute to a state of functional exhaustion of CD8+ T-cells in the TME, as demonstrated by PD-1 expression (45). PD-1 has an immunoreceptor tyrosine-based switch motif that binds the Src homology region 2 domain-containing phosphatase, SHP-2, which is responsible for the dephosphorylation of post-TCR signaling molecules such as CD3ζ, ZAP70, Akt, and ERK (8). Dephosphorylation results in interference in TCR signal transduction, culminating in reduced expression of genes that are involved in CTL activation and tumor cell killing. Instead, the cellular response deviates towards the expression of an “exhaustion gene program,” which contributes to additional inefficiency to provide tumor cell killing (46). Against this background, Taylor et al. discovered that siRNA knockdown or inhibition of GSK3 in murine CD8⁺ T-cells was capable of reducing the cell surface expression of PD-1, in addition to boosting cytolytic killing of a lymphomatous tumor (13). Moreover, it was demonstrated that these interventions increased the transcriptional activation of the Tbx21 promoter, leading to increased T-bet expression and transcriptional suppression of the PD-1 promoter in CTLs (10). Chromatin immunoprecipitation assays further confirmed that GSK3 inhibition increased T-bet association with the Pdcd1 promoter. Surface expression of PD-1 allows T-cell interaction with PD-L1, which is expressed on cancer and stromal cells in the TME. To reverse the effects of T-cell exhaustion and to restore cytotoxic tumor cell killing, it is possible to intervene in the inhibitory effects of the PD-1/PD-L1 axis, either through the use of checkpoint receptor blocking antibodies (8, 47) or utilizing the ability of GSK-3 inhibitors to interfere in PD-1 expression. For the sake of completeness, it is also important to mention that PD-1 impacts T-cell activation by APC, allowing anti-PD1 or GSK3 inhibitors to impact the afferent events leading to the generation of cytotoxic and helper T-cells that shape anti-tumor immune responses (13, 48).

While inhibition of PD-1/PD-L1 is an attractive therapeutic target, the use of monoclonal antibodies (mAbs) to block PD-1/PD-L1 has potential pitfalls. mAbs are large bio-reactive proteins and interact with a number of other biomolecules, proteins, and cell surfaces after systemic administration, including FcRn and Fcγ receptors (5). These interactions and the hydrodynamic size of the antibodies can constrain the PK/PD profiles relative to small molecules (49, 50). Moreover, even fully humanized mAbs are potentially immunogenic (sometimes toxic) and can result in the production of anti-IgG antibodies that could lead to rapid antibody clearance (6, 51, 52). mAbs are also expensive and difficult to manufacture. While SMIs capable of directly blocking the binding of PD-1 to PD-L1 have had some success in preclinical studies (53), small molecule inhibitors of GSK3 has not as yet achieved FDA approval or advanced to clinical treatment (11, 15). Moreover, even for the most-advanced GSK3 inhibitor, LY2090314 (a Phase 2 candidate), the clinical trial was abandoned due to the poor PK and drug bioavailability (clinical trial ID: NCT01214603, NCT01632306) (54). Another concern for the use of SMI of GSK3 include off-target effects due to the pleiotropic role of this signaling hub kinase (55). For instance, in one preclinical study, GSK3β disruption led to embryonic lethality in mice, generating a phenotype similar to the disruption of the IKKβ gene in the NF-κB pathway (56). Moreover, the favorable characteristic of AZD1080 for crossing the blood-brain barrier to treat Alzheimer's disease (29), was offset by the development of nephrotoxicity in a phase I clinical trial (15). Thus, from the perspective of treatment side effects, encapsulation of GSK3 inhibitors in a nanocarrier may be advantageous by reducing systemic biodistribution in exchange for increase drug delivery to the tumor site, where their action is required. The development of silicasomes, characterized by high drug loading capacity, circulatory stability, and excellent biodistribution to the heterogeneous tumor sites, introduces a highly effective nanocarrier to improve the efficacy of AZD1080 delivery, in addition to the lack of any observable toxicity.

The ability of the silicasome carrier for AZD1080 to inhibit growth in four syngeneic mouse cancer models by interfering in PD-1 expression demonstrates its utility as a possible replacement for antibody-based immune checkpoint blockade monotherapy. While sAZD1080 and anti-PD-1 monotherapy were effective in the MC38 colon and LLC lung cancer models, only sAZD1080 (but not anti-PD-1) could inhibit CT26 and KPC tumor growth. The KPC model, which carries a point mutation in p53 gene (TP53^{R172H}) and a point mutation in the KRAS gene (KRAS^{G12D}), is generally recognized as poorly responsive to therapy by immune checkpoint blocking antibodies, similar to the findings in its human counterpart (40). While there are several reasons for poor responsiveness, the dysplastic PDAC stroma plays an important role in drug resistance by restricting vascular access or drug catabolism (57). In spite of these challenges, we demonstrate the efficacy of the silicasome platform in sAZD1080 delivery, similar to what we have previously shown for irinotecan, gemcitabine and paclitaxel delivery to the PDAC site (19, 22, 25). This includes delivery of 6% of injected drug dose at the KPC tumor site, in addition to the possibility to further improve delivery by inducing blood vessel transcytosis with a cyclic iRGD peptide (19-22, 25).

Anti-PD-1 antibodies (Pembrolizumab, Nivolumab) have been approved as immunotherapies for the use in a number of solid tumors, such as gastric cancer, hepatocellular carcinoma, head and neck squamous cell carcinoma, urothelial carcinoma, cervical cancer, non-small cell lung cancer, and broadly for non-respectable solid tumors with high microsatellite instability (MSI-H) or DNA mismatch repair deficiency (58). It is conceivable, based on our results, that sAZD1080 would be effective in the treatment of these cancers as a monotherapy. Moreover, a growing trend is to use immune checkpoint blockade in combination with chemotherapy, radiotherapy, or other targeted therapies. According to clinicaltrials.gov, there are currently ˜870 active clinical studies involving anti-PD-1 for anti-PD-L1 treatment, 60% of which is premised on combination therapy. Anti-PD-1 treatment is FDA approved for combination therapy with paclitaxel or oxaliplatin for treatment of melanoma and biliary tract cancer, pemetrexed for non-small cell lung cancer and axitinib for renal cell carcinoma (9, 59-62). Moreover, the heterogeneous immune landscapes across multiple cancer types hold the promise of combination therapy, premised on the “hot” or “cold” immune status of the tumor, as well as multiple immune escape pathways operating in the TME. Thus, one type of intervention could be to convert “cold” into “hot” tumors by specific chemotherapeutic agents, radiotherapy, or photodynamic therapy, which is known to induce immunogenic cell death (ICD) (63-65). The newly acquired anti-tumor immunity can then be propagated through the use of immune checkpoint interference. This can be approached by the designing a “2-in-1” nanocarrier strategy that co-encapsulates and ICD-inducing chemo agent (i.e. doxorubicin, oxaliplatin, irinotecan, mitoxantrone, paclitaxel, cyclophosphamide, etc.) with AZD1080. This is accomplishable by selecting weak-basic, amphipathic drugs for remote loading. Another option is to select an ICD-inducing chemotherapeutic agent such as paclitaxel (66) for loading into the silicasome lipid membrane, followed by remote loading of AZD1080 in the porous interior. In this regard, we have previously shown the utility of the silicasome for co-delivery of paclitaxel (from the lipid bilayer) and gemcitabine (from the porous interior) in PDAC (67). Alternatively, we could design a lipid conjugated prodrug that allows the GSK3 inhibitor to be incorporated into the silicasome lipid bilayer, allowing and ICD-inducing chemotherapeutic agent to be remotely imported into the porous interior.

While sAZD1080, have been shown to be highly effective in four syngeneic cancer models, a limitation of this study is that tumor growth was established by subcutaneous implanting. There are differences between subcutaneous and orthotopic models of the same cancer type, which could lead to immunotherapy response differences, including in response to checkpoint blocking antibodies (68). Unfortunately, there is no consistency in the literature regarding prediction making of the immunotherapy response, as exemplified by the contradictory results of the response outcomes in syngeneic colon cancer models during immune checkpoint therapy (69, 70). Irrespective of these inconsistencies, the reproducible response outcomes in four subcutaneous models treated with sAZD1080, is a strong incentive to proceed with orthotopic experimentation, which more likely recapitulate human disease (71).

In summary, silicasome nanoparticle delivery of the GSK3 inhibitor, AZD1080, was effective at inhibiting tumor growth in four syngeneic mouse cancer models through the pharmacological inhibition of PD-1 expression in T-cells. This marks a significant advancement in the nanoparticle-based delivery of a small molecule for inhibition of immune checkpoint pathways in cancer, including for the supplementation or replacement of anti-PD-1/PD-L1 antibodies and treatment combinations.

### Methods

Materials

All GSK3 inhibitors were purchased from Cayman Chemical (Ann Arbor, Mich.). The following antibodies for flow cytometry were purchased from BioLegend (San Diego, Calif.): BV510 anti-CD45 (cat 103137), AF647 anti-CD8a (cat 100727), BV785 anti-NK1.1 (cat 108749), BV711 anti-CD107a (cat 121631), BV421 anti-CD279 (cat 135217), and PE anti-granzyme B antibody (cat 372207). Chemical reagents for MSNP synthesis were purchased from Sigma Aldrich (St. Louis, Mo.), as described previously (22). 18:0 DSPC (cat 850365), 18:0 PEG2000 PE (cat 880120), and cholesterol (cat 700100) for silicasome synthesis were purchased from Avanti Polar Lipids (Alabaster, Ala.).

Use of Medicinal Chemistry Criteria to Identify GSK3 Inhibitors for Carrier Remote Loading

Commercially available GSK3 inhibitors were analyzed using MarvinSketch software (ChemAxon, Budapest, Hungary) to evaluate the chemical properties of the compounds listed in FIG. 10. This includes calculating the partitioning (cLogP) and solubility coefficients (logS) at pH 7.4. IC₅₀ values were obtained from Cayman Chemical (www.caymanchem.com/) and Selleck Chemicals (www.selleckchem.com/).

Silicasome Preparation for Encapsulation of a Trapping Agent

Mesoporous silica nanoparticles (MSNPs) were synthesized as a large batch, as previously described (22). Briefly, this involves the addition of 0.9 L of 25 wt % CTAC in water to 17.1 L pure water in a beaker, stirred at 85° C. 72 g triethanolamine was added, followed by 600 mL TEOS. After stirring for 4 hours and cooling to room temperature, the bear MSNPs were precipitated with ethanol and CTAC was removed by washing in acidic ethanol, with sonication. MSNPs at 80 mg/mL in ethanol were centrifuged at 21,000×g for 15 minutes to pellet the nanoparticles. After removal of the ethanol supernatant, the MSNP pellet was resuspended in 123 mM ammonium sulfate in water by bath sonication. 40 mg of MSNP was resuspended in 1 mL of 123 mM ammonium sulfate. Materials for the lipid bilayer were dissolved in ethanol to provide a molar ratio of 60:40:3 for DPSC, cholesterol, and PE-PEG₂₀₀₀. Altogether, this amounted to 120 mg lipid (3× the MSNP mass), which was dissolved in 240 μL, ethanol at 65° C. The aqueous MSNP suspension was rapidly added to the lipid solution, followed by dilution with 5 mL of 123 mM ammonium sulfate. The crude lipid/MSNP mixture was probe sonicated at 40% intensity, using two rounds of pulsing (each for 10 seconds, 5 second pause, 5 minutes pulsing). The silicasome/liposomes mixture was centrifuged for 5 minutes at 5,000×g to pellet large aggregates. The supernatant was collected and centrifuged at 21,000×g for 15 minutes to pellet the silicasomes. After washing and repeat of the centrifugation step, silicasomes were resuspended in 0.9% NaCl solution.

Comparative Analysis of Drug Loading Capacity and Efficiency of GSK3 Inhibitor Silicasomes

Four GSK3 inhibitor-laden silicasome formulations were analyzed to assess encapsulation efficiency and loading capacity. 100 μL of the silicasomes suspension at 10 mg/mL in 0.9% NaCl (1 mg silicasomes) was added to AZD1080, AZD2858, LY2090314, and 1-azakenpaullone solutions, which were prepared by adding 50, 100 or 200 μg of each inhibitor to 900 μL water. These drug mass quantities provided feed weight percentages of 5, 10, and 20%, in comparison to the silicasome mass. Following execution of the remote loading procedure as detailed above, the crude solutions were used to assess absorbance in a glass bottom 96-well plate (Total Drug Absorbance). Silicasomes were centrifuged at 21,000×g for 15 minutes to pellet the particles. After removal of the supernatant, the silicasomes were re-suspended in aa fresh 0.9% NaCl solution. This was repeated three times to remove any unloaded inhibitor. The silicasome solution was then sampled and absorbance was measured to determine Loaded Drug Absorbance. Concentration-matched unloaded ‘blank’ silicasomes also had absorbance measured (Silicasome Blank Absorbance). Encapsulation efficiency was calculated by the following formula:

$$\frac{L oaded Drug Absorbance - S i l i c asome Blank Absorbance}{Total Drug Absorbance - Silicasome Blank Absorbance} \times 100 \%$$

All measurements were taken using a SpectraMax M5 plate reader (Molecular Devices). All solutions were diluted 1/10 in 0.9% NaCl solution prior to absorbance reads. The absorbances used for each compound were: 418 nm for AZD1080, 383 nm for AZD2858, 213 nm for LY2090314, and 334 nm for 1-azakenpaullone.

Characterization of AZD1080-Laden Silicasome

Bare MSNPs were characterized by transmission electron microscopy (JEOL 1200-EX). Bare and AZD1080-laden silicasomes were characterized by cryogenic transmission electron microscopy (cryoTEM, TF20 FEI Tecnai-G2). Silicasomes were prepared at 100 μg/mL in PBS and were analyzed for hydrodynamic diameter and zeta potential, using dynamic light scattering in a benchtop Zetasizer (Brookhaven).

Real Time RT-qPCR in Cultured Primary Murine T-Cells

Wells of a 96-well tissue culture plate were coated with anti-mouse CD3 antibody (Ultra-LEAF, Biolegend). 7.5 μL of 1 mg/mL antibody solution was diluted in 1 mL of 1×PBS, before 50 μL of the dilution was added to each plate well for 2 hours at 37° C. The wells were then thoroughly washed 3× with 1×PBS.

T-cells were extracted from C57BL/6 mouse spleens, using a standard procedure with minor modifications (71). Two mouse spleens were mechanically disrupted and the released cells were passed through a 70 μm filter. Red blood cells were lysed using RBC lysis buffer (eBioscience) at 4° C. for 5 minutes. 10 mL of 1×PBS was added to stop the lysis process, following which cells were pelleted at 300×g for 5 minutes. Cells were resuspended in 1 mL of Mojosort buffer, and negative, T-cell selection proceeded as per the manufacturer's instructions (BioLegend, MojoSort Mouse CD3 T-cell Isolation Kit). Briefly, splenocytes were incubated with a negative selection antibody cocktail (biotin anti-Gr-1, biotin anti-B220, biotin anti-CD49b, biotin anti-CD19, biotin anti-CD11b, biotin anti-CD24, biotin anti-TER-119). Following the addition of streptavidin magnetic nanobeads, the cell suspension was incubated within the Mojosort magnet for 5 minutes to aggregate and capture non-T-cells. The still-suspended T-cells were decanted from the test tube and were washed. After cell counting and pelleting at 300×g for 5 min, cells were resuspended in RPMI+10% FBS+pen/strep+glutamax to a concentration of 1.2×10⁶ cells/mL. 200 μL cells (˜2.4×10⁵ cells) were added to each well of the anti-CD3 coated plate. 10 μL of each of the inhibitor compounds, solubilized in 10% DMSO/PBS, were added to each well to reach final concentration of 10 μM. 10 μL of 10% DMSO without any inhibitor was used as a control. Cells were incubated in the anti-CD3 coated plates together with the inhibitors for 72 hours. All treatments were performed in quadruplicate, and the experiment was reproduced twice.

Cells were collected and RNA was extracted using Direct-zol RNA MiniPrep Plus kits (Zymo), as per the manufacturer's instructions. Extracted RNA was converted into cDNA using SuperScript III First-Strand Synthesis System kits (Invitrogen). cDNA from treated T-cells was analyzed by RT-PCR using a LightCycler 480 instrument (Roche) and the LightCycler 480 SYBR Green I Master kit (Roche), per manufacturer's instructions. cDNA was analyzed using forward and reverse primers for Pdcd1, Tbx21, and Gapdh as a control, as described previously (10, 14). This included use of the following primers:

tbet-fw-
(SEQ ID NO: 16)
5′-GATCGTCCTGCAGTCTCTCC-3′,
tbet-rv
(SEQ ID NO: 17)
5′-AACTGTGTTCCCGAGGTGTC-3′,
pdcd1-fw
(SEQ ID NO: 18)
5′-CCGCCTTCTGTAATGGTTTGA-3′,
pdcd1-rv
(SEQ ID NO: 19)
5′-GGGCAGCTGTATGATCTGGAA-3′,
gapdh-fw
(SEQ ID NO: 20)
5′-CAACAGCAACTCCCACTCTTC-3′,
and
gapdh-rv
(SEQ ID NO: 21)
5′-GGTCCAGGGTTTCTTACTCCTT-3 (ThermoFisher)

Pdcd1 and Tbx21 expression were normalized against Gapdh expression.

Animals

Animal care was executed according to the “Principles of Laboratory Animal Care” of the National Society for Medical Research (USA). The experimental protocol was approved by Animal Research Committee at University of California, Los Angeles. All mice (female) were purchased from Charles River at an age of 6-8 weeks old.

Maximum Tolerated Dose Study

The maximum tolerated dose of AZD1080 was determined using a protocol from the National Cancer Institute (72). C57BL/6 mice were injected with AZD1080 solutions via tail vein (100 μL) and weights were recorded daily for 14 days. AZD1080 was solubilized in 30% PEG400, 0.5% Tween-80, 5% polyethylene, and 64.5% 1×PBS. The injected amounts of AZD1080 were 5, 7.5, 11.25, 16.87, or 25.31 mg/kg per mouse, n=4 mice per group.

Use of the MC38 Syngeneic Colon Cancer Model to Assess the Effect of GSK3 Inhibitors

MC38 cells, stably transfected with a luciferase vector, were inoculated into the right flank of C57BL/6 mice, using 0.7×10⁶ cells in 40% Matrigel 1×PBS solution. Tumors were allowed to grow to a size of ˜50 mm³ prior to the initiation of treatment, approximately 14 days after initial inoculation. Mice with outlier tumor sizes (either too small or too large) were excluded from further study. The remaining animals were randomly assigned to the different treatment groups. Mice were treated with three injections, set three days apart, and were sacrificed 4 days after the final treatment administration. Mice were either treated with saline (IV), aPD-1 (4 mg/kg/mouse; IP), sAZD1080 (5 mg/kg/mouse AZD1080; IV), free AZD1080 in vehicle solution (30% PEG400, 0.5% Tween-80, 5% polyethylene, 64.5% 1×PBS) (5 mg/kg/mouse AZD1080; IP), or free SB415286 in vehicle solution (8 mg/kg/mouse; IP). Two separate experiments were performed, the first with n=6 mice per group and the second with n=9 animals.

Flow Cytometry

Tumors were harvested from MC38 mice after sacrifice. Tumors larger than 100 mm³ were divided in two, with one half preserved for histology and the other half used to conduct flow cytometry. To prepare samples for flow cytometry, tumor chunks of 2-3 mm³ were incubated in an enzyme cocktail (1 mg/mL collagenase type IV, 2000 U DNase type IV, 0.1 mg/mL hyaluronidase type V in 1×HBSS) for 1 hour, and the digests passed through a 70 μm nylon filter. Cells were pelleted and washed in HBSS.

Cells were incubated in Zombie NIR dye (BioLegend) for 10 minutes, and then treated with the following antibodies against T-cell surface antigens: BV510, anti-CD45 (1:80); AF647, anti-CD8a (1:400); BV785, anti-NK1.1 (1:300); BV711, anti-CD107a (1:50); and BV421, anti-CD279 (1:40) for 15 minutes. Cells were diluted in cell staining buffer, pelleted by centrifuging at 350×g for 5 minutes, and washed twice. Cell pellets were resuspended in 1×permeabilization buffer (Biolegend), before incubation with PE-labeled anti-granzyme B antibody (1:40) for 20 minutes. After washing and resuspension in cell staining buffer, flow cytometric analysis was performed in a BD Fortessa flow cytometer.

Single-stained cells and single-color beads were used for construction of a compensation matrix, which was applied to flow cytometry data prior to gating and analysis. Analysis was performed using the online flow cytometry software Cytobank (https://community.cytobank.org/cytobank).

Tissue Histology

The histology preparation and staining of tumor tissue and mouse organs was performed in the Pathology Core Facility of the UCLA Jonsson Comprehensive Cancer Center. Tissues were fixed in 10% neutral-buffered formalin for 24 hours, then gradually dehydrated in 25-75% ethanol solutions over 3 days. Tissues were embedded in paraffin, sectioned at 4 μm thickness, and stained by anti-CD8, anti-cleaved caspase 3, anti-perforin, anti-PD-1, or haemotoxylin and eosin (H&E). Slides were digitally scanned for analysis using Aperio ImageScope software (Leica Biosystems, Buffalo Grove, Ill.). Four fields of view were analyzed per tumor at 20× magnification, with all positively stained cells counted within the field and averaged across fields. The investigator was blinded with respect to the animal groups to avoid bias during counting. Fields of view were chosen to avoid the tumor periphery and processing artifacts.

Blood Chemistry Panel

At the time of sacrifice, blood was obtained by cardiac puncture from MC38 tumor growing animals and collected into Greiner Bio-One MiniCollect Capillary Blood collection system tubes. Tubes were centrifuged as per manufacturer's instructions to separate serum from blood cells. The serum was frozen at −20 and subsequently used, to obtain a comprehensive serum chemistry panel, develop by IDEXX Laboratories (Westbrook, Me.), for use in the UCLA Division of Laboratory Animal Medicine (DLAM).

Studying the Impact of AZD1080 in Additional Syngeneic Cancer Models

Three other subcutaneous cancer models were examined: KPC, CT26, and Lewis Lung Carcinoma (LLC). While CT26 and LLC cells were obtained from commercial sources, the immortalized KPC cell line was derived from a spontaneous primary tumor growing in a transgenic Kras^{LSL-G12D/+}; Trp53^{LSL-R172H/+}; Pdx-1-Cre mouse (21). KPC cells were subcutaneously injected in syngeneic B6/129 mice, CT26 cells into syngeneic BALB/c mice, and LLC cells into syngeneic C57BL/6 mice. Each animal received 1×10⁶ cells in 40% Matrigel in PBS into the right flank. Tumors were allowed to grow to a size of ˜100 mm³ prior to the initiation of treatment, typically 10-11 days after inoculation. Animals with outlier tumor sizes (either too small or too large) were excluded from further study, and the remaining mice were randomly assigned to the treatment groups. Mice were treated with three injections, delivered three days apart, and were sacrificed 4 days after administration of the final treatment. Mice were either treated with saline IV, aPD-1 (4 mg/kg/mouse; IP), or sAZD1080 (5 mg/kg/mouse AZD1080; IV). For the KPC model, the number of animals per group were: 10 for saline, 8 for aPD1, and 7 for sAZD1080. For the CT26 model, the n values were: 9 for saline, 6 for aPD1, and 6 for sAZD1080. For the LLC model, the n values were: 9 for saline, 7 for aPD1, and 7 for sAZD1080.

Statistics

Comparative analysis of the differences between groups was performed using Brown-Forsythe ANOVA and Dunnett's T3 multiple comparisons test (GraphPad Prism 8.4.1). Values were expressed as mean±standard deviation, unless otherwise stated within the figure legends. For all statistical analyses, p<0.05 was considered statistically significant.

### REFERENCES

1. Haslam A, and Prasad V. Estimation of the Percentage of U S Patients With Cancer Who Are Eligible for and Respond to Checkpoint Inhibitor Immunotherapy Drugs. JAMA Netw Open. 2019; 2(5):e192535.
2. Jerby-Arnon L, Shah P, Cuoco M S, Rodman C, Su M J, Melms J C, Leeson R, Kanodia A, Mei S, Lin J R, et al. A Cancer Cell Program Promotes T Cell Exclusion and Resistance to Checkpoint Blockade. Cell. 2018; 175(4):984-97 e24.
3. Umansky V, Blattner C, Gebhardt C, and Utikal J. The Role of Myeloid-Derived Suppressor Cells (MDSC) in Cancer Progression. Vaccines (Basel). 2016; 4(4).
4. Marin-Acevedo J A, Dholaria B, Soyano A E, Knutson K L, Chumsri S, and Lou Y. Next generation of immune checkpoint therapy in cancer: new developments and challenges. J Hematol Oncol. 2018; 11(1):39.
5. Zhao L, Ren T H, and Wang D D. Clinical pharmacology considerations in biologics development. Acta Pharmacol Sin. 2012; 33(11):1339-47.
6. Descotes J. Immunotoxicity of monoclonal antibodies. MAbs. 2009; 1(2):104-11.
7. Dolan C. Opportunities and challenges in biosimilar uptake in oncology. Am J Manag Care. 2018; 24(11 Suppl): S237-S43.
8. Mizuno R, Sugiura D, Shimizu K, Maruhashi T, Watada M, Okazaki I M, and Okazaki T. PD-1 Primarily Targets TCR Signal in the Inhibition of Functional T Cell Activation. Front Immunol. 2019; 10(630.
9. Seidel J A, Otsuka A, and Kabashima K. Anti-PD-1 and Anti-CTLA-4 Therapies in Cancer: Mechanisms of Action, Efficacy, and Limitations. Front Oncol. 2018; 8(86.
10. Taylor A, Harker J A, Chanthong K, Stevenson P G, Zuniga E I, and Rudd C E. Glycogen Synthase Kinase 3 Inactivation Drives T-bet-Mediated Downregulation of Co-receptor PD-1 to Enhance CD8(+) Cytolytic T Cell Responses. Immunity. 2016; 44(2):274-86.
11. Saraswati A P, Ali Hussaini S M, Krishna N H, Babu B N, and Kamal A. Glycogen synthase kinase-3 and its inhibitors: Potential target for various therapeutic conditions. Eur J Med Chem. 2018; 144(843-58.
12. Declercq L D, Vandenberghe R, Van Laere K, Verbruggen A, and Bormans G. Drug Development in Alzheimer's Disease: The Contribution of PET and SPECT. Front Pharmacol. 2016; 7(88.
13. Taylor A, and Rudd C E. Glycogen Synthase Kinase 3 Inactivation Compensates for the Lack of CD28 in the Priming of CD8(+) Cytotoxic T-Cells: Implications for anti-PD-1 Immunotherapy. Front Immunol. 2017; 8(1653.
14. Taylor A, Rothstein D, and Rudd C E. Small-Molecule Inhibition of PD-1 Transcription Is an Effective Alternative to Antibody Blockade in Cancer Therapy. Cancer Res. 2018; 78(3):706-17.
15. Eldar-Finkelman H, and Martinez A. GSK-3 Inhibitors: Preclinical and Clinical Focus on CNS. Front Mol Neurosci. 2011; 4(32.
16. Ugolkov A, Gaisina I, Zhang J S, Billadeau D D, White K, Kozikowski A, Jain S, Cristofanilli M, Giles F, O'Halloran T, et al. GSK-3 inhibition overcomes chemoresistance in human breast cancer. Cancer Lett. 2016; 380(2):384-92.
17. Ding L, Madamsetty V S, Kiers S, Alekhina O, Ugolkov A, Dube J, Zhang Y, Zhang J S, Wang E, Dutta S K, et al. Glycogen Synthase Kinase-3 Inhibition Sensitizes Pancreatic Cancer Cells to Chemotherapy by Abrogating the TopBP1/ATR-Mediated DNA Damage Response. Clin Cancer Res. 2019; 25(21):6452-62.
18. O'Flaherty L, Shnyder S D, Cooper P A, Cross S J, Wakefield J G, Pardo O E, Seckl M J, and Tavare J M. Tumor growth suppression using a combination of taxol-based therapy and GSK3 inhibition in non-small cell lung cancer. PLoS One. 2019; 14(4):e0214610.
19. Meng H, Wang M, Liu H, Liu X, Situ A, Wu B, Ji Z, Chang C H, and Nel A E. Use of a lipid-coated mesoporous silica nanoparticle platform for synergistic gemcitabine and paclitaxel delivery to human pancreatic cancer in mice. ACS Nano. 2015; 9(4):3540-57.
20. Lu J, Liu X, Liao Y P, Salazar F, Sun B, Jiang W, Chang C H, Jiang J, Wang X, Wu A M, et al. Nano-enabled pancreas cancer immunotherapy using immunogenic cell death and reversing immunosuppression. Nat Commun. 2017; 8(1):1811.
21. Liu X, Lin P, Perrett I, Lin J, Liao Y P, Chang C H, Jiang J, Wu N, Donahue T, Wainberg Z, et al. Tumor-penetrating peptide enhances transcytosis of silicasome-based chemotherapy for pancreatic cancer. J Clin Invest. 2017; 127(5):2007-18.
22. Liu X, Jiang J, Chan R, Ji Y, Lu J, Liao Y P, Okene M, Lin J, Lin P, Chang C H, et al. Improved Efficacy and Reduced Toxicity Using a Custom-Designed Irinotecan-Delivering Silicasome for Orthotopic Colon Cancer. ACS Nano. 2019; 13(1):38-53.
23. Cern A, Golbraikh A, Sedykh A, Tropsha A, Barenholz Y, and Goldblum A. Quantitative structure-property relationship modeling of remote liposome loading of drugs. J Control Release. 2012; 160(2):147-57.
24. Messerer C L, Ramsay E C, Waterhouse D, Ng R, Simms E M, Harasym N, Tardi P, Mayer L D, and Bally M B. Liposomal irinotecan: formulation development and therapeutic assessment in murine xenograft models of colorectal cancer. Clin Cancer Res. 2004; 10(19):6638-49.
25. Liu X, Situ A, Kang Y, Villabroza K R, Liao Y, Chang C H, Donahue T, Nel A E, and Meng H. Irinotecan Delivery by Lipid-Coated Mesoporous Silica Nanoparticles Shows Improved Efficacy and Safety over Liposomes for Pancreatic Cancer. ACS Nano. 2016; 10(2):2702-15.
26. Haran G, Cohen R, Bar L K, and Barenholz Y. Transmembrane ammonium sulfate gradients in liposomes produce efficient and stable entrapment of amphipathic weak bases. Biochim Biophys Acta. 1993; 1151(2):201-15.
27. Cern A, Barenholz Y, Tropsha A, and Goldblum A. Computer-aided design of liposomal drugs: In silico prediction and experimental validation of drug candidates for liposomal remote loading. J Control Release. 2014; 173(125-31.
28. Bolotin E M, Cohen R, Bar L K, Emanuel N, Ninio S, Barenholz Y, and Lasic D D. Ammonium Sulfate Gradients for Efficient and Stable Remote Loading of Amphipathic Weak Bases into Liposomes and Ligandoliposomes. Journal of Liposome Research. 1994; 4(1):455-79.
29. Georgievska B, Sandin J, Doherty J, Mortberg A, Neelissen J, Andersson A, Gruber S, Nilsson Y, Schott P, Arvidsson P I, et al. AZD1080, a novel GSK3 inhibitor, rescues synaptic plasticity deficits in rodent brain and exhibits peripheral target engagement in humans. J Neurochem. 2013; 125(3):446-56.
30. Aktas E, Kucuksezer U C, Bilgic S, Erten G, and Deniz G. Relationship between CD107a expression and cytotoxic activity. Cellular Immunology. 2009; 254(2):149-54.
31. Kansy B A, Concha-Benavente F, Srivastava R M, Jie H B, Shayan G, Lei Y, Moskovitz J, Moy J, Li J, Brandau S, et al. PD-1 Status in CD8(+) T Cells Associates with Survival and Anti-PD-1 Therapeutic Outcomes in Head and Neck Cancer. Cancer Res. 2017; 77(22):6353-64.
32. Chattopadhyay P K, Betts M R, Price D A, Gostick E, Horton H, Roederer M, and De Rosa S C. The cytolytic enzymes granyzme A, granzyme B, and perforin: expression patterns, cell distribution, and their relationship to cell maturity and bright CD57 expression. J Leukoc Biol. 2009; 85(1):88-97.
33. Brennan A J, Chia J, Trapani J A, and Voskoboinik I. Perforin deficiency and susceptibility to cancer. Cell Death Differ. 2010; 17(4):607-15.
34. Larimer B M, Bloch E, Nesti S, Austin E E, Wehrenberg-Klee E, Boland G, and Mahmood U. The Effectiveness of Checkpoint Inhibitor Combinations and Administration Timing Can Be Measured by Granzyme B PET Imaging. Clin Cancer Res. 2019; 25(4):1196-205.
35. Homet Moreno B, Zaretsky J M, Garcia-Diaz A, Tsoi J, Parisi G, Robert L, Meeth K, Ndoye A, Bosenberg M, Weeraratna A T, et al. Response to Programmed Cell Death-1 Blockade in a Murine Melanoma Syngeneic Model Requires Costimulation, CD4, and CD8 T Cells. Cancer immunology research. 2016; 4(10):845-57.
36. Mosely S I, Prime J E, Sainson R C, Koopmann J O, Wang D Y, Greenawalt D M, Ahdesmaki M J, Leyland R, Mullins S, Pacelli L, et al. Rational Selection of Syngeneic Preclinical Tumor Models for Immunotherapeutic Drug Discovery. Cancer Immunol Res. 2017; 5(1):29-41.
37. Li H Y, McSharry M, Bullock B, Nguyen T T, Kwak J, Poczobutt J M, Sippel T R, Heasley L E, Weiser-Evans M C, Clambey E T, et al. The Tumor Microenvironment Regulates Sensitivity of Murine Lung Tumors to PD-1/PD-L1 Antibody Blockade. Cancer Immunol Res. 2017; 5(9):767-77.
38. Gopinathan A, Morton J P, Jodrell D I, and Sansom O J. GEMMs as preclinical models for testing pancreatic cancer therapies. Disease Models &amp;amp; Mechanisms. 2015; 8(10):1185.
39. Lee J W, Komar C A, Bengsch F, Graham K, and Beatty G L. Genetically Engineered Mouse Models of Pancreatic Cancer: The KPC Model (LSL-Kras(G12D/+);LSL-Trp53(R172H/+);Pdx-1-Cre), Its Variants, and Their Application in Immuno-oncology Drug Discovery. Curr Protoc Pharmacol. 2016; 73(14 39 1-14 39 20.
40. Hingorani S R, Wang L, Multani A S, Combs C, Deramaudt T B, Hruban R H, Rustgi A K, Chang S, and Tuveson D A. Trp53R172H and KrasG12D cooperate to promote chromosomal instability and widely metastatic pancreatic ductal adenocarcinoma in mice. Cancer Cell. 2005; 7(5):469-83.
41. Kabacaoglu D, Ciecielski K J, Ruess D A, and Algul H. Immune Checkpoint Inhibition for Pancreatic Ductal Adenocarcinoma: Current Limitations and Future Options. Front Immunol. 2018; 9(1878.
42. Steele C W, Karim S A, Leach J D G, Bailey P, Upstill-Goddard R, Rishi L, Foth M, Bryson S, McDaid K, Wilson Z, et al. CXCR2 Inhibition Profoundly Suppresses Metastases and Augments Immunotherapy in Pancreatic Ductal Adenocarcinoma. Cancer Cell. 2016; 29(6):832-45.
43. Zitvogel L, and Kroemer G. Targeting PD-1/PD-L1 interactions for cancer immunotherapy. Oncoimmunology. 2012; 1(8):1223-5.
44. Isaaz S, Baetz K, Olsen K, Podack E, and Griffiths G M. Serial killing by cytotoxic T lymphocytes: T cell receptor triggers degranulation, re-filling of the lytic granules and secretion of lytic proteins via a non-granule pathway. Eur J Immunol. 1995; 25(4):1071-9.
45. Jiang Y, Li Y, and Zhu B. T-cell exhaustion in the tumor microenvironment. Cell Death Dis. 2015; 6(e1792.
46. Simon S, and Labarriere N. PD-1 expression on tumor-specific T cells: Friend or foe for immunotherapy? Oncoimmunology. 2017; 7(1):e1364828.
47. Wang S, He Z, Wang X, Li H, and Liu X S. Antigen presentation and tumor immunogenicity in cancer immunotherapy response prediction. Elife. 2019; 8(
48. Jenkins M K, Taylor P S, Norton S D, and Urdahl K B. CD28 delivers a costimulatory signal involved in antigen-specific IL-2 production by human T cells. J Immunol. 1991; 147(8):2461-6.
49. Castelli M S, McGonigle P, and Hornby P J. The pharmacology and therapeutic applications of monoclonal antibodies. Pharmacol Res Perspect. 2019; 7(6):e00535.
50. Lobo E D, Hansen R J, and Balthasar J P. Antibody pharmacokinetics and pharmacodynamics. J Pharm Sci. 2004; 93(11):2645-68.
51. Garces S, and Demengeot J. The Immunogenicity of Biologic Therapies. Curr Probl Dermatol. 2018; 53(37-48.
52. Ovacik M, and Lin K. Tutorial on Monoclonal Antibody Pharmacokinetics and Its Considerations in Early Development. Clin Transl Sci. 2018; 11(6):540-52.
53. Ganesan A, Ahmed M, Okoye I, Arutyunova E, Babu D, Turnbull W L, Kundu J K, Shields J, Agopsowicz K C, Xu L, et al. Comprehensive in vitro characterization of PD-L1 small molecule inhibitors. Sci Rep. 2019; 9(1):12392.
54. Rizzieri D A, Cooley S, Odenike O, Moonan L, Chow K H, Jackson K, Wang X, Brail L, and Borthakur G. An open-label phase 2 study of glycogen synthase kinase-3 inhibitor LY2090314 in patients with acute leukemia. Leuk Lymphoma. 2016; 57(8):1800-6.
55. Hu S, Begum A N, Jones M R, Oh M S, Beech W K, Beech B H, Yang F, Chen P, Ubeda O J, Kim P C, et al. GSK3 inhibitors show benefits in an Alzheimer's disease (AD) model of neurodegeneration but adverse effects in control animals. Neurobiol Dis. 2009; 33(2):193-206.
56. Hoeflich K P, Luo J, Rubie E A, Tsao M S, Jin O, and Woodgett J R. Requirement for glycogen synthase kinase-3beta in cell survival and NF-kappaB activation. Nature. 2000; 406(6791):86-90.
57. Neesse A, Michl P, Frese K K, Feig C, Cook N, Jacobetz M A, Lolkema M P, Buchholz M, Olive K P, Gress T M, et al. Stromal biology and therapy in pancreatic cancer. Gut. 2011; 60(6):861-8.
58. Wu X, Gu Z, Chen Y, Chen B, Chen W, Weng L, and Liu X. Application of PD-1 Blockade in Cancer Immunotherapy. Comput Struct Biotechnol J. 2019; 17(661-74.
59. Sun D, Ma J, Wang J, Han C, Qian Y, Chen G, Li X, Zhang J, Cui P, Du W, et al. Anti-PD-1 therapy combined with chemotherapy in patients with advanced biliary tract cancer. Cancer. Immunol Immunother. 2019; 68 (9): 1527-35.
60. Burtness B, Harrington K J, Greil R, Soulieres D, Tahara M, de Castro G, Jr., Psyrri A, Baste N, Neupane P, Bratland A, et al. Pembrolizumab alone or with chemotherapy versus cetuximab with chemotherapy for recurrent or metastatic squamous cell carcinoma of the head and neck (KEYNOTE-048): a randomised, open-label, phase 3 study. Lancet. 2019; 394(10212):1915-28.
61. Langer C J, Gadgeel S M, Borghaei H, Papadimitrakopoulou V A, Patnaik A, Powell S F, Gentzler R D, Martins R G, Stevenson J P, Jalal S I, et al. Carboplatin and pemetrexed with or without pembrolizumab for advanced, non-squamous non-small-cell lung cancer: a randomised, phase 2 cohort of the open-label KEYNOTE-021 study. The Lancet Oncology. 2016; 17(11):1497-508.
62. Atkins M B, Plimack E R, Puzanov I, Fishman M N, McDermott D F, Cho D C, Vaishampayan U, George S, Olencki T E, Tarazi J C, et al. Axitinib in combination with pembrolizumab in patients with advanced renal cell cancer: a non-randomised, open-label, dose-finding, and dose-expansion phase 1 b trial. Lancet Oncol. 2018; 19(3):405-15.
63. Bezu L, Gomes-de-Silva L C, Dewitte H, Breckpot K, Fucikova J, Spisek R, Galluzzi L, Kepp O, and Kroemer G. Combinatorial strategies for the induction of immunogenic cell death. Front Immunol. 2015; 6(187.
64. Rapoport B L, and Anderson R. Realizing the Clinical Potential of Immunogenic Cell Death in Cancer Chemotherapy and Radiotherapy. Int J Mol Sci. 2019; 20(4).
65. Golden E B, and Apetoh L. Radiotherapy and immunogenic cell death. Semin Radiat Oncol. 2015; 25(1):11-7.
66. Wang Y J, Fletcher R, Yu J, and Zhang L. Immunogenic effects of chemotherapy-induced tumor cell death. Genes Dis. 2018; 5(3):194-203.
67. Von Hoff D D, Ramanathan R K, Borad M J, Laheru D A, Smith L S, Wood T E, Korn R L, Desai N, Trieu V, Iglesias J L, et al. Gemcitabine plus nab-paclitaxel is an active regimen in patients with advanced pancreatic cancer: a phase I/II trial. J Clin Oncol. 2011; 29(34):4548-54.
68. Hage C, Hoves S, Ashoff M, Schandl V, Hort S, Rieder N, Heichinger C, Berrera M, Ries C H, Kiessling F, et al. Characterizing responsive and refractory orthotopic mouse models of hepatocellular carcinoma in cancer immunotherapy. PLoS One. 2019; 14(7):e0219517.
69. Zhao X, Li L, Starr T K, and Subramanian S. Tumor location impacts immune response in mouse models of colon cancer. Oncotarget. 2017; 8(33):54775-87.
70. Song W, Tiruthani K, Wang Y, Shen L, Hu M, Dorosheva O, Qiu K, Kinghorn K A, Liu R, and Huang L. Trapping of Lipopolysaccharide to Promote Immunotherapy against Colorectal Cancer and Attenuate Liver Metastasis. Adv Mater. 2018; 30(52):e1805007.
71. Lim J F, Berger H, and Su I H. Isolation and Activation of Murine Lymphocytes. J Vis Exp. 2016116).
72. Aston W J, Hope D E, Nowak A K, Robinson B W, Lake R A, and Lesterhuis W J. A systematic investigation of the maximum tolerated dose of cytotoxic chemotherapy with and without supportive care in mice. BMC Cancer. 2017; 17(1):684.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A drug delivery vehicle for the delivery of a GSK3 inhibitor, wherein:
said drug delivery vehicle comprises:
a nanoparticle comprising one or more cavities disposed within said nanoparticle and an outside surface where said one or more cavities are in fluid communication the outside surface of said nanoparticle;
a GSK3 inhibitor disposed within said one or more cavities; and
a lipid bilayer disposed on the surface of said nanoparticle where said lipid bilayer fully encapsulates said nanoparticle; or
said drug delivery vehicle comprises:
a solid nanoparticle;
a GSK3 inhibitor disposed on the surface of said nanoparticle and/or intermixed within said nanoparticle; and
a lipid bilayer disposed on the surface of said nanoparticle where said lipid bilayer fully encapsulates said nanoparticle; or
said drug delivery vehicle comprises a liposome comprising a lipid bilayer.

2. The drug delivery vehicle of claim 1, wherein said GSK3 inhibitor comprises a weak basic GS3K inhibitor.

3. The drug delivery vehicle of claim 2, wherein said GS3K inhibitor is selected from the group consisting of AZD2858, AZD1080, LY2090314, and 1-Azakenpaullone.

4. The drug delivery vehicle of claim 3, wherein said GS3K inhibitor comprises AZD1080.

5. The drug delivery vehicle according to any one of claims 1-4, wherein said drug delivery vehicle comprises:
a nanoparticle comprising one or more cavities disposed within said nanoparticle and an outside surface where said one or more cavities are in fluid communication the outside surface of said nanoparticle;
a GSK3 inhibitor disposed within said one or more cavities; and
a lipid bilayer disposed on the surface of said nanoparticle where said lipid bilayer fully encapsulates said nanoparticle.

6. The drug delivery vehicle of claim 5, said nanoparticle comprise a single cavity.

7. The drug delivery vehicle of claim 6, wherein said nanoparticle comprises a nanobowl.

8. The drug delivery vehicle of claim 6, wherein said nanoparticle comprises a hollow nanosphere.

9. The drug delivery vehicle of claim 5, wherein said nanoparticle comprises a plurality of cavities.

10. The drug delivery vehicle of claim 9, wherein said nanoparticle comprises a porous inorganic nanoparticle, a metal-organic framework nanoparticle, or a porous organic nanoparticle.

11. The drug delivery vehicle of claim 10, wherein said nanoparticle comprise a porous inorganic nanoparticle.

12. The drug delivery vehicle of claim 11, wherein said nanoparticle comprise a porous silica nanoparticle, a porous calcium carbonate nanoparticle, a porous carbon nanoparticle, a hollow core Fe₃O₄ nanoparticle, or a porous calcium phosphate nanoparticle.

13. The drug delivery vehicle of claim 12, wherein said nanoparticle comprises a porous silica nanoparticle.

14. The drug delivery vehicle of claim 13, wherein said nanoparticle comprises a mesoporous silica nanoparticle (MSN), a mesoporous organosilica nanoparticle (MONs), or a periodic mesoporous organosilica (PMO) nanoparticle.

15. The drug delivery vehicle of claim 14, wherein said nanoparticle comprises a mesoporous silica nanoparticle (MSN).

16. The drug delivery vehicle of claim 15, wherein said nanoparticle comprises undoped and unfunctionalized silica.

17. The drug delivery vehicle according to any one of claims 14-15, wherein said nanoparticle comprises a mesoporous silica/hydroxyapatite (MSNs/HAP) hybrid nanoparticle.

18. The drug delivery vehicle according to any one of claims 14-15, wherein said nanoparticle comprises a cleavable silsesquioxane, or a bridged silsesquioxane (BS).

19. The drug delivery vehicle according to any one of claims 14-15, wherein said nanoparticle comprises an inorganically doped silica.

20. The drug delivery vehicle of claim 19, wherein said nanoparticle comprises a calcium-, iron-, manganese-, or zirconium-doped silica.

21. The drug delivery vehicle according to any one of claims 14-15, wherein said nanoparticle comprises an imine-doped silica.

22. The drug delivery vehicle of claim 12, wherein said nanoparticle comprises a mesoporous calcium carbonate nanoparticle.

23. The drug delivery vehicle of claim 12, wherein said nanoparticle comprises a mesoporous calcium phosphate nanoparticle.

24. The drug delivery vehicle of claim 10, wherein said nanoparticle comprises a porous biocompatible polymer.

25. The drug delivery vehicle of claim 24, wherein said nanoparticle comprise a porous biocompatible polymer selected from the group consisting of polymers of the polyaryletherketone (PAEK) family (e.g., polyether ether ketone (PEEK), carbon reinforced PEEK, polyether ketone ketone (PEKK), PEKEKK (polyetherketoneetherketoneketone), polyaryletherketone (PAEK), polyetherketone (PEK), Polyetherketone Etherketone Ketone (PEKEKK), and the like), polycaprolactone (PCL), polylactic acid (PLA), polyglycolic acid (PGA), polyphenylene, self-reinforced polyphenylene, polyphenylsulphone, poly sulphone, polyethylene terephthalate (PET), polyethylene, polyurethane, oligocarbonatedimethacrylate (OCM-2) porous polymer, carbonate- and phthalate-containing dimethacrylates, and the like.

26. The drug delivery vehicle of claim 24, wherein said nanoparticle comprises a hydrogel.

27. The drug delivery vehicle of claim 26, wherein said hydrogel comprises a hydrogel formed from one or more materials selected from the group consisting of poly(N-isopropylacrylamide) (PNIPA), poly(N-isopropylacrylamide-co-1-vinylimidazole) (PNIPA-VI), poly(acrylamide) (PAAm), poly(acrylamide), poly(N,N-dimethylacrylamide), poly(N,N-diethylacrylamide), poly(1-vinylimidazole), poly(sodium acrylate), poly(sodium methacrylate), poly(2-hydroxyethylmethacrylate) (HEMA), poly(N,N-dimethylaminoethyl methacrylate) (DMAEMA), poly(N-[tris(hydroxymethyl)methyl]acrylamide), poly(1-(3-methacryloxy)propylsulfonic acid) (sodium salt), poly(allylamine), poly(N-acryloxysuccinimide), poly(N-vinylcaprolactam), poly(1-vinyl-2-pyrrolidone), poly(2-acrylamido-2-methyl-1-propanesulfonic acid) (sodium salt), poly((3-acrylamidopropyl) trimethylammonium chloride), and poly(diallyldimethylammonium chloride).

28. The drug delivery vehicle of claim 10, wherein said nanoparticle comprises a metal organic framework (MOF).

29. The drug delivery vehicle of claim 28, wherein said nanoparticle comprises a metal organic framework selected from the group consisting of zeolitic imidazolate frameworks (ZIFs), Universitetet i Oslo (University of Oslo) frameworks (UiOs), and (Materials of Institut Lavoisier frameworks (MILs).

30. The drug delivery vehicle of claim 29, wherein said nanoparticle comprises a metal organic framework selected from the group consisting of ZIF-8, ZIF-67, ZIF-90, Fe-BTC, HKUST-1, and MEL-53, MIL-89, MEL-88A, MIL-100, UiO-66, UiO-66-NH₂, MOF-801, MOF-804, Fe-NDC-M, MOF-1201, MOF-1203, and Fe-NDC-O MOFs.

31. The drug delivery vehicle of claim 30, wherein said nanoparticle comprises a MEL-88A MOF.

32. The drug delivery vehicle of claim 30, wherein said nanoparticle comprises a ZIF-8 MOF.

33. The drug delivery vehicle of claim 30, wherein said nanoparticle comprises a UiO-66 MOF, or a UiO-66-NH₂ MOF.

34. The drug delivery vehicle according to any one of claims 1-33, wherein said nanoparticle has an average pore size that ranges from about 1 to about 20 nm, or from about 1 to about 10 nm, or from about 1 to about 5 nm, or from about 1 to about 4 nm, or from about 1 to about 3 nm, or from about 2 to about 3 nm.

35. The drug delivery vehicle according to any one of claims 1-4, wherein said drug delivery vehicle comprises:
a solid nanoparticle;
a GSK3 inhibitor disposed on the surface of said nanoparticle and/or intermixed within said nanoparticle; and
a lipid bilayer disposed on the surface of said nanoparticle where said lipid bilayer fully encapsulates said nanoparticle.

36. The drug delivery vehicle of claim 35, wherein said nanoparticle is selected from the group consisting of a metal nanoparticle, or a biocompatible polymer nanoparticle.

37. The drug delivery vehicle of claim 36, wherein said nanoparticle comprises a zinc oxide nanoparticle, a gold nanoparticle, a silver nanoparticle, an aluminum hydroxide nanoparticle, a silica nanoparticle, a core-shell Fe₃O—SiO₂ nanoparticle, a core-shell NaYbF4:Tm-NaYF₄ upconverting nanoparticle, or a LiYF₄:Tm/Yb nanocrystal.

38. The drug delivery vehicle of claim 36, wherein said nanoparticle comprises a biocompatible polymer.

39. The drug delivery vehicle of claim 38, wherein said nanoparticle comprises a biocompatible polymer selected from the group consisting of poly(lactic-co-glycolic acid) (PLGA), Poly(glycolic acid) (PGA), Poly(lactic acid) (PLA), Poly(caprolactone) (PCL), Poly(butylene succinate), Poly(trimethylene carbonate), Poly(p-dioxanone), Poly(butylene terephthalate), Poly(ester aminde) (HYBRANE®), polyurethane, Poly[(carboxyphenoxy) propane-sebacic acid], Poly[bis(hydroxyethyl) terephthalate-ethyl orthophosphorylate/terephthaloyl chloride], Poly(β-hydroxyalkanoate), Poly(hydroxybutyrate), Poly(hydroxybutyrate-co-hydroxyvalerate), and chitosan.

40. The drug delivery vehicle according to any one of claims 1-4, wherein said drug delivery vehicle comprises a liposome that comprises a lipid bilayer.

41. The drug delivery vehicle according to any one of claims 1-40, wherein said drug delivery vehicles have an average hydrodynamic diameter ranging from about 30 nm up to about 300 nm, or from about 30 up to about 200 nm, or from about 30 up to about 170 nm, or from about 30 nm up to about 150 nm, or from about 30 up to about 100 nm, or from about 30 up to about 80 nm, or from about 30 up to about 70 nm, or from about 40 up to about 70 nm by DLS.

42. The drug delivery vehicle of claim 41, wherein said drug delivery vehicles have an average hydrodynamic diameter ranging from about 70 nm up to about 165 nm by DLS by DLS.

43. The nanoparticle drug carrier according to any one of claims 1-42, wherein said lipid bilayer comprises a phospholipid, and cholesterol (CHOL) and/or a cholesterol derivative.

44. The nanoparticle drug carrier of claim 43, wherein said lipid bilayer comprises a phospholipid and cholesterol (CHOL).

45. The nanoparticle drug carrier according to any one of claims 43-44, wherein said phospholipid comprises a saturated fatty acid with a C14-C20 carbon chain, and/or an unsaturated fatty acid with a C14-C20 carbon chain, and/or a natural lipid comprising a mixture of fatty acids with C12-C20 carbon chains.

46. The nanoparticle drug carrier of claim 45, wherein said phospholipid comprises one or more phospholipids selected from the group consisting of distearoylphosphatidylcholine (DSPC), phosphatidylcholine (DPPC), 1,2-dimyristoleoyl-sn-glycero-3-phosphocholine (DMPC), 1,2-dioleoyl-sn-glycero-3-phosphocholine (DOPC), 1,2-distearoyl-sn-glycero-3-phospho-rac-glycerol (DSPG), 1,2-dipalmitoyl-sn-glycero-3-phosphoglycerol (DPPG), 1,2-dieicosenoyl-sn-glycero-3-phosphocholine, and diactylphosphatidylcholine (DAPC), and dipalmitoyl phosphatidylethanolamine.

47. The nanoparticle drug carrier of claim 45, wherein said phospholipid comprises a natural lipid selected from the group consisting of egg phosphatidylcholine (egg PC), and soy phosphatidylcholine (soy PC).

48. The nanoparticle drug carrier of claim 45, wherein said phospholipid comprises distearoylphosphatidylcholine (DSPC).

49. The nanoparticle drug carrier according to any one of claims 43-48, wherein said lipid bilayer comprises an mPEG phospholipid with a phospholipid C14-C18 carbon chain, and a PEG molecular weight ranging from about 350 Da to 5000 Da.

50. The nanoparticle drug carrier of claim 49, wherein said lipid bilayer comprises dipalmitoyl phosphatidylethanolamine grafted poly(ethylene glycol) (PE-PEG).

51. The nanoparticle drug carrier of claim 50, wherein said PE-PEG comprises PE-PEG_{2K}.

52. The nanoparticle drug carrier of claim 50, wherein said PE-PEG comprises PE-PEG_{5K}.

53. The nanoparticle drug carrier according to any one of claims 48-52, wherein said lipid bilayer comprises DPSC, cholesterol, and PE-PEG.

54. The nanoparticle drug carrier of claim 53, wherein the ratio of DPSC:cholesterol:PE-PEG ranges from 40-90% DSPC:10%-50% Chol:1%-10% PE-PEG (molar ratio).

55. The nanoparticle drug carrier of claim 54, wherein the ratio of DSPC:Chol:PE-PEG is about 60:40:3 molar ratio.

56. The nanoparticle drug carrier according to any one of claims 43-55, wherein said lipid bilayer comprises a cholesterol derivative selected from the group consisting of cholesterol hemisuccinate (CHEMS), lysine-based cholesterol (CHLYS), and PEGylated cholesterol (Chol-PEG).

57. The nanoparticle drug carrier of claim 56, wherein said lipid bilayer comprises CHEMS.

58. The nanoparticle drug carrier of claim 57, wherein said bilayer comprises CHEMS ranging from about 5% (mol percent) up to about 30% total lipid.

59. The nanoparticle drug carrier of claim 58, wherein said bilayer comprises about 10% or about 20% CHEMS or about 30% CHEMS or about 40% CHEMS.

60. The nanoparticle drug carrier according to any one of claims 1-42, wherein said lipid bilayer comprises a formulation shown in Table 3.

61. The nanoparticle drug carrier according to any one of claims 1-60, wherein the GSK3 inhibitor is loaded with a cargo trapping agent (e.g., protonating agent).

62. The nanoparticle drug carrier of claim 61, wherein said cargo trapping agent before reaction with the GSK3 inhibitor loaded in the nanoparticle, is selected from the group consisting of citric acid, triethylammonium sucrose octasulfate (TEA₈SOS), (NH₄)₂SO₄, an ammonium salt, a trimethylammonium salt, and a triethylammonium salt.

63. The nanovesicle drug carrier of claim 62, wherein said cargo-trapping agent before reaction with said drug is ammonium sulfate.

64. The nanoparticle drug carrier according to any one of claims 1-63, wherein said drug carrier comprises an additional therapeutic agent disposed inside of the nanoparticle.

65. The nanoparticle drug carrier of claim 64, wherein said additional therapeutic agent comprises an inducer of immunogenic cell death (ICD inducer).

66. The nanoparticle drug carrier of claim 65, wherein said ICD inducer comprises a chemotherapeutic agent selected from the group consisting of irinotecan, doxorubicin, oxaliplatin, anthracenedione, bleomycin, bortezomib, cisplatin, daunorubicin, docetaxel, epirubicin, idarubicin, mitoxanthrone, paclitaxel, 82016, and cyclophosphamide.

67. The nanoparticle drug carrier of claim 66, wherein said ICD inducer comprises irinotecan.

68. The nanoparticle drug carrier of claim 64, wherein said additional therapeutic agent comprise an anticancer agent selected from the group consisting of doxorubicin, epirubicin, pirarubicin, daunorubicin, rubidomycin, valrubicin, amrubicin, irinotecan, topotecan, 10-hydroxycamptothecin, belotecan, rubitecan, vinorelbine, LAQ824, vinblastine, vincristine, homoharringtonine, trabectedin, mitoxantrone, cyclophosphamide, mechlorethamine, temozolomide, 5-fluorouracil, 5′-deoxy-5-fluorouridine, gemcitabine, capecitabine, pazopanib, enzastaurin, vandetanib erlotinib, dasatinib, nilotinib, sunitinib, osimertinib, palbociclib, and ribociclib.

69. The nanoparticle drug carrier of claim 68, wherein said additional therapeutic agent comprises mitoxantrone.

70. The nanoparticle drug carrier of claim 68, wherein said additional therapeutic agent comprises gemcitabine.

71. The nanoparticle drug carrier of claim 68, wherein said additional therapeutic agent comprises doxorubicin.

72. The nanoparticle drug carrier according to any one of claims 1-71, wherein said drug carrier comprises a hydrophobic therapeutic agent disposed in the lipid bilayer.

73. The nanoparticle drug carrier of claim 71, wherein said hydrophobic therapeutic agent comprises paclitaxel.

74. The nanoparticle drug carrier according to any one of claims 1-73, wherein said drug carrier is conjugated to a moiety selected from the group consisting of a targeting moiety, a fusogenic peptide, and a transport peptide.

75. The nanoparticle drug carrier of claim 74, wherein said drug carrier is conjugated to a peptide that binds a receptor on a cancer cell or tumor blood vessel.

76. The nanoparticle drug carrier of claim 75, wherein said drug carrier is conjugated to an iRGD peptide.

77. The nanoparticle drug carrier of claim 75, wherein said drug carrier is conjugated to a targeting ligand shown in Table 5.

78. The nanoparticle drug carrier according to any one of claims 74-77, wherein said drug carrier is conjugated to transferrin, and/or ApoE, and/or folate.

79. The nanoparticle drug carrier according to any one of claims 74-78, wherein said drug carrier is conjugated to a targeting moiety that comprises an antibody that binds to a cancer marker.

80. The nanoparticle drug carrier of claim 79, wherein said drug carrier is conjugated to a targeting moiety that comprises an antibody that binds a cancer marker shown in Table 4.

81. The nanoparticle drug carrier according to any one of claims 79-80, wherein said antibody is selected from the group consisting of an intact immunoglobulin, an F(ab)′₂, a Fab, a single chain antibody, a diabody, an affibody, a unibody, and a nanobody.

82. The nanoparticle drug carrier according to any one of claims 1-81, wherein said drug carriers in suspension are stable for at least 1 month, or at least 2 months, or at least 3 months, or at least 4 months, or at least 5 months, or at least 6 months when stored at 4° C.

83. The nanoparticle drug carrier according to any one of claims 1-82, wherein said nanoparticle drug carrier forms a stable suspension on rehydration after lyophilization.

84. The nanoparticle drug carrier according to any one of claims 1-83, wherein said nanoparticle drug carriers, show reduced drug toxicity as compared to free GSK3 inhibitor.

85. The nanoparticle drug carrier according to any one of claims 1-84, wherein said nanoparticle drug carrier has colloidal stability in physiological fluids with pH 7.4 and remains monodisperse to allow systemic biodistribution and is capable of entering a disease site by vascular leakage (EPR effect) or transcytosis.

86. The nanoparticle drug carrier drug carrier according to any one of claims 1-85, wherein said carrier is colloidally stable.

87. A pharmaceutical formulation comprising:
nanoparticle drug carrier according to any one of claims 1-86; and
a pharmaceutically acceptable carrier.

88. The pharmaceutical formulation of claim 87, wherein said formulation is an emulsion, dispersion, or suspension.

89. The pharmaceutical formulation of claim 88, wherein said suspension, emulsion, or dispersion is stable for at least 1 month, or at least 2 months, or at least 3 months, or at least 4 months, or at least 5 months, or at least 6 months when stored at 4° C.

90. The pharmaceutical formulation according to any one of claims 87-89, wherein the nanovesicle drug carriers, and/or the a nanoparticle drug carriers, and/or the a nanomaterial carriers in said formulation show a substantially unimodal size distribution; and/or show a PDI less than about 0.2, or less than about 0.1.

91. The pharmaceutical formulation according to any one of claims 87-90, wherein said formulation is formulated for administration via a route selected from the group consisting of intravenous administration, intraarterial administration, intracerebral administration, intrathecal administration, oral administration, aerosol administration, administration via inhalation (including intranasal and intratracheal delivery, intracranial administration via a cannula, and subcutaneous or intramuscular depot deposition.

92. The pharmaceutical formulation according to any one of claims 87-90, wherein said formulation is a sterile injectable.

93. The pharmaceutical formulation according to any one of claims 87-92, wherein said formulation is a unit dosage formulation.

94. A method of treating a cancer, said method comprising:
administering to a subject in need thereof an effective amount of a nanoparticle drug carrier according to any one of claims 1-86; and/or
a pharmaceutical formulation according to any one of claims 87-93.

95. The method of claim 94, wherein said method comprises a component of a primary therapy in a chemotherapeutic regimen.

96. The method of claim 94, wherein said method comprises an adjunct therapy in a treatment regime that additionally comprises chemotherapy using another chemotherapeutic agent, and/or surgical resection of a tumor mass, and/or radiotherapy.

97. The method according to any one of claims 94-96, wherein said composition, a nanovesicle drug carrier, a nanoparticle drug carrier according, and/or nanomaterial carrier is a component in a multi-drug chemotherapeutic regimen.

98. The method according to any one of claims 94-97, wherein said cancer comprises a solid tumor.

99. The method of claim 98, wherein said cancer comprises a cancer selected from the group consisting of gastric cancer, hepatocellular carcinoma, head and neck squamous cell carcinoma, urothelial carcinoma, cervical cancer, non-small cell lung cancer, and broadly for non-respectable solid tumors with high microsatellite instability (MSI-H) or DNA mismatch repair deficiency.

100. The method according to any one of claims 94-97, wherein said cancer comprises pancreatic cancer.

101. The method according to any one of claims 94-97, wherein said cancer comprises colorectal cancer.

102. The method according to any one of claims 94-97, wherein said cancer comprises lung cancer.

103. The method according to any one of claims 94-97, wherein said cancer is a cancer selected from the group consisting of breast cancer, lung cancer, melanoma, pancreas cancer, liver cancer, acute lymphoblastic leukemia (ALL), acute myeloid leukemia (AML), adrenocortical carcinoma, AIDS-related cancers (e.g., Kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, brain and spinal cord tumors, brain stem glioma, central nervous system atypical teratoid/rhabdoid tumor, central nervous system embryonal tumors, central nervous system germ cell tumors, craniopharyngioma, ependymoma, burkitt lymphoma, carcinoid tumors (e.g., childhood, gastrointestinal), cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm's tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sézary syndrome), Hodgkin, non-Hodgkin, primary central nervous system (CNS)), macroglobulinemia, Waldenström, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, Myelogenous Leukemia, Chronic (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

104. The method according to any one of claims 94-103, wherein said administration is via a route selected from the group consisting of intravenous administration, intraarterial administration, intracerebral administration, intrathecal administration, oral administration, aerosol administration, administration via inhalation (including intranasal and intratracheal delivery, intracranial administration via a cannula, and subcutaneous or intramuscular depot deposition.

105. The method according to any one of claims 94-103, wherein said administration comprises systemic administration via injection or cannula.

106. The method according to any one of claims 94-103, wherein said administration is administration to an intra-tumoral or peri-tumoral site.

107. The method according to any one of claims 94-106, wherein said mammal is a human.

108. The method according to any one of claims 94-106, wherein said mammal is a non-human mammal.
